(19) United States
(12) Patent Application Publication
(10) Pub. No.: US 2021/0369768 A1
(43) Pub. Date: Dec. 2, 2021

(54) METAL DI-AMINO ACID CHELATES OR METAL TRI-AMINO ACID CHELATES

(71) Applicant: Balchem Corporation, New Hampton, NY (US)

(72) Inventors: Robert Doyle, Syracuse, NY (US); Ren Gonzalez, New Hampton, NY (US)

(21) Appl. No.: 17/335,772

(22) Filed: Jun. 1, 2021

Related U.S. Application Data
(60) Provisional application No. 63/032,955, filed on Jun. 1, 2020.
(60) Provisional application No. 63/152,136, filed on Feb. 22, 2021.

(51) Int. Cl. A61K 33/06; A61K 45/06

(52) U.S. Cl. CPC A61K 33/06; A61K 45/06

(57) ABSTRACT

The present disclosure relates to metal di-amino acid chelates and metal tri-amino acid chelates.

### CROSS-REFERENCE TO RELATED APPLICATIONS

This application claims priority to U.S. Provisional Application No. 63/032,955, filed Jun. 1, 2020, entitled “METAL TRI-AMINO ACID CHELATES,” and U.S. Provisional Application No. 63/152,136, filed Feb. 22, 2021, entitled “METAL DI-AMINO ACID CHELATES OR METAL TRI-AMINO ACID CHELATES,” which are incorporated by reference herein in their entirety.

### FIELD OF THE DISCLOSURE

The present disclosure relates to metal di-amino acid or tri-amino acid chelates.

### BACKGROUND OF THE DISCLOSURE

Mineral deficiency is a lack of the dietary minerals necessary for an organism's proper health. Deficiencies may be caused by a poor diet, impaired uptake of the minerals that are consumed or a dysfunction in the organism's use of the mineral after it is absorbed. When mineral deficiencies do occur, they can result in significant health issues.

For example, magnesium is a critical mineral, involved in ˜80% of known metabolic functions in humans. It is estimated that 45%-60% of people worldwide are magnesium deficient, a condition associated with disease states like hypertension, diabetes, and neurological disorders, to name a few. Magnesium deficiency may be due to be dietary practices, medications, and farming techniques, along with estimates that the mineral content of vegetables has declined by as much as 80-90% in the last 100 years.

Similarly, zinc deficiency affects the skin and gastrointestinal tract; brain and central nervous system, immune, skeletal, and reproductive systems, while calcium deficiency leads to low bone mass and risk for osteoporosis. A severe calcium deficiency can even produce symptoms such as numbness and tingling in the fingers, convulsions and abnormal heart rhythm.

Metal chelates have been explored as a means of correcting mineral deficiencies when administered to subjects. Known metal chelates, however, have drawbacks such as difficult synthesis reactions, or poor in vivo bioavailability. There is a need in the art for simple, cost-effective metal chelates that provide minerals in a bioavailable form to subjects.

### SUMMARY OF THE DISCLOSURE

Disclosed herein are compositions comprising a magnesium di-amino acid chelate complex, the complex comprising a magnesium di-amino acid cheate and a counterion, wherein there is a 1:1 ratio between the magnesium and the di-amino acid. The di-amino acid may be selected from the group consisting of di-glycine (G2), di-aspartic acid (D2), di-glutamic acid (E2), di-histidine (H2), di-serine (S2), and di-tyrosine (Y2), or each amino acid of the di-amino acid may be selected from the group consisting of glycine (G), aspartic acid (D), glutamic acid (E), histidine (H), serine (S), and tyrosine (Y). In one example, the di-amino acid is di-glycine. The composition may further comprise at least one vitamin or additional mineral.

Further disclosed herein are compositions comprising a zinc di-amino acid chelate complex, the complex comprising a magnesium di-amino acid cheate and optionally a counterion, wherein there is a 1:1 or 1:2 ratio between the zinc and the di-amino acid. The di-amino acid may be selected from the group consisting of di-glycine (G2), di-aspartic acid (D2), di-glutamic acid (E2), di-histidine (H2), di-serine (S2), and di-tyrosine (Y2), or each amino acid of the di-amino acid may be selected from the group consisting of glycine (G), aspartic acid (D), glutamic acid (E), histidine (H), serine (S), and tyrosine (Y). In one example, the di-amino acid is di-glycine. The composition may further comprise at least one vitamin or additional mineral.

Further disclosed herein are compositions comprising a calcium di-amino acid chelate complex, the complex comprising a calcium di-amino acid cheate and optionally a counterion, wherein there is a 1:1 or 1:2 ratio between the calcium and the di-amino acid. The di-amino acid may be selected from the group consisting of di-glycine (G2), di-aspartic acid (D2), di-glutamic acid (E2), di-histidine (H2), di-serine (S2), and di-tyrosine (Y2), or each amino acid of the di-amino acid may be selected from the group consisting of glycine (G), aspartic acid (D), glutamic acid (E), histidine (H), serine (S), and tyrosine (Y). In one example, the di-amino acid is di-glycine. The composition may further comprise at least one vitamin or additional mineral.

Also disclosed herein are pharmaceutical formulations comprising any of the compositions described above. The pharmaceutical formulation may be suitable for oral or parenteral administration.

The present disclosure also includes a method of supplying a metal to a subject deficient in a metal comprising administering to the subject any of the compositions described above. The metal may include magnesium, calcium, iron, zinc, or combinations thereof.

The present disclosure also includes a method of producing a metal di-amino acid chelate complex comprising (i) creating an aqueous solution of di-amino acid, a metal compound, and optionally an organic acid; (ii) stirring he solution of step (i) while optionally heating up to 90° C. for a minimum of 10 minutes; and (iii) cooling the solution and precipitating the metal di-amino acid chelate complex. The method may also include individually heating an aqueous solution of the metal compound and optionally the organic acid before combining them to create the solution of step (i).

The present disclosure also includes a metal di-amino acid chelate complex formed by the method comprising (i) creating an aqueous solution of di-amino acid, a metal compound, and optionally an organic acid; (ii) stirring he solution of step (i) while optionally heating up to 90° C. for a minimum of 10 minutes; and (iii) cooling the solution and precipitating the metal di-amino acid chelate complex.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts an illustration of a structure of a metal tri-amino acid chelate of the present disclosure. Specifically, FIG. 1 illustrates a magnesium 2-[[2-[(2-aminoacetyl)amino]acetyl]amino]acetic acid (also called Glycylglycylglycine) chelate.

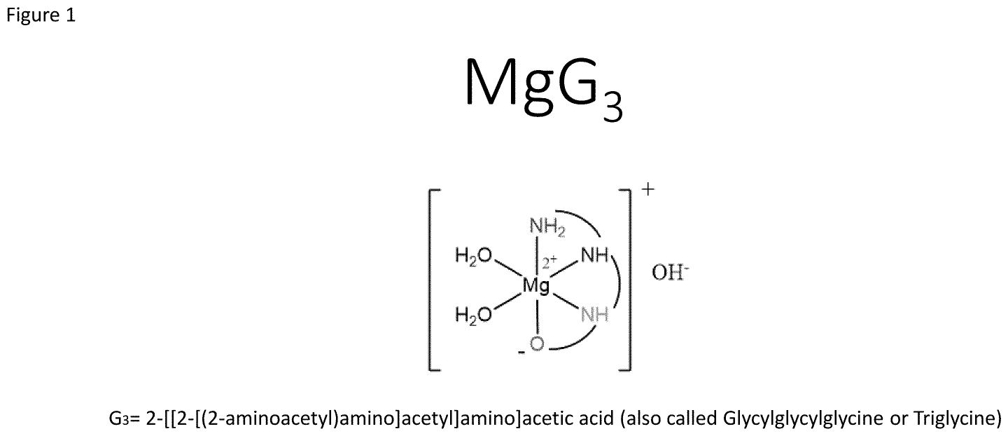

FIG. 2 illustrates a method of producing a metal tri-amino acid chelate. Specifically, FIG. 2 illustrates a method of producing a magnesium 2-[[2-[(2-aminoacetyl)amino]acetyl]amino]acetic acid (also called Glycylglycylglycine) chelate. A 1.0025 g sample of triglycine (G3—5.29 mmol; 1 eq.) was dissolved in approximately 10 mLs DI H₂O in a 50 mL round-bottom flask, with constant heating and stirring at 90° C. A separate solution of 215.5 mgs magnesium oxide (MgO—5.29 mmol; 1 eq.) was taken up in approximately 10 mLs DI H₂O, with an addition of 253.6 mgs citric acid (0.25 eq), constantly stirred and heated to 90° C. The Mg/CA solution was added to the triglycine solution—upon addition, the combined solution turned a milky, white color (after 20 minutes, the solution was wholly soluble). The reaction was left to run for 2 hrs at 90° C. The reaction was cooled to room temperature and filtered through a Buchner funnel (no solid was observed on the filter paper). The pH of the solution was found to be 10.2. The solution was concentrated down to approximately 3 mLs and the solid was precipitated with ethanol. Centrifugation (4,000 rpm; 10 mins; RT) was employed to pellet the solid, and the ethanol was decanted off. The solid was washed with diethyl ether to remove ethanol, the sample was then recentrifuged, the ether decanted off, and the solid dried in vacuo overnight. The dried material was collected and massed. Yield was found to be nearly stoichiometric.

FIG. 3 depicts the characterization of a Mg(G3) reaction supernatant via ESI-MS. The expected masses are G3=189 (m/z), Mg(G3)=211 (m/z), Mg(G3).H₂O=229 (m/z), and [Mg(G3).H₂O.Na]¹⁺=251 (m/z). The appropriate isotopic distribution pattern was observed (Inset right). The ESI-MS was run in H₂O/MeOH. The presence of both the free G3 ligand as well as the chelate G3 ligand is seen. The Inset at the right exhibits the appropriate isotopic distribution pattern of a magnesium chelate.

FIG. 4 depicts the characterization of Mg(G3) via FT-IR.

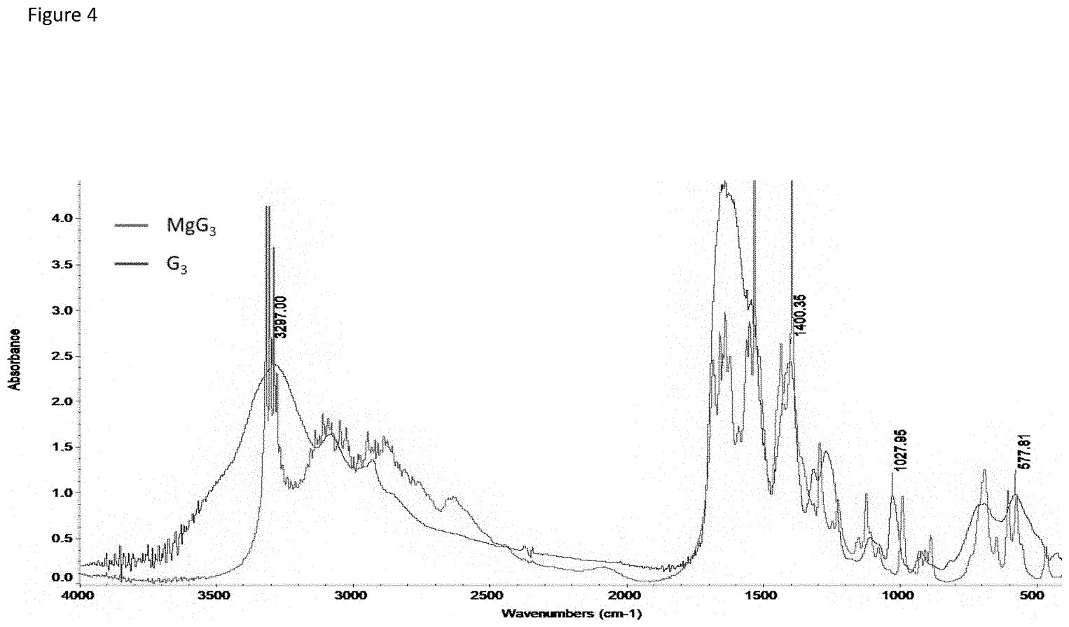

FIG. 5 depicts the characterization of G3 via ¹H NMR. The expected integration—6 (2:2:2); The observed integration—6 (2:2:2). NMR was taken in D₂O.

FIG. 6 depicts the characterization of Mg(G3) via ¹H NMR (2 hrs). The total observed integration remains at 6. No subsequent splitting change of G3 singlets suggesting the lack or presence of isomers. Upfield shift of H₁ (0.5 ppm), Upfield shift of H₂ (0.1 ppm), and Upfield shift of H₃ (0.04 ppm). NMR taken in D₂O.

FIG. 7 depicts a ¹H NMR overlay of G3 and Mg(G3) (2 hrs).

FIG. 8 depicts G3, Mg(G3), and Mg citrate (MgCit) ¹H NMR Overlay (2 hrs). All NMR were taken in D₂O. The upfield shift of proton peaks in Mg(G3) suggests G3 chelation to magnesium. ¹H NMR of Mg(G3) reaction run in citrate buffer suggests that citrate will out compete G3for chelation to magnesium.

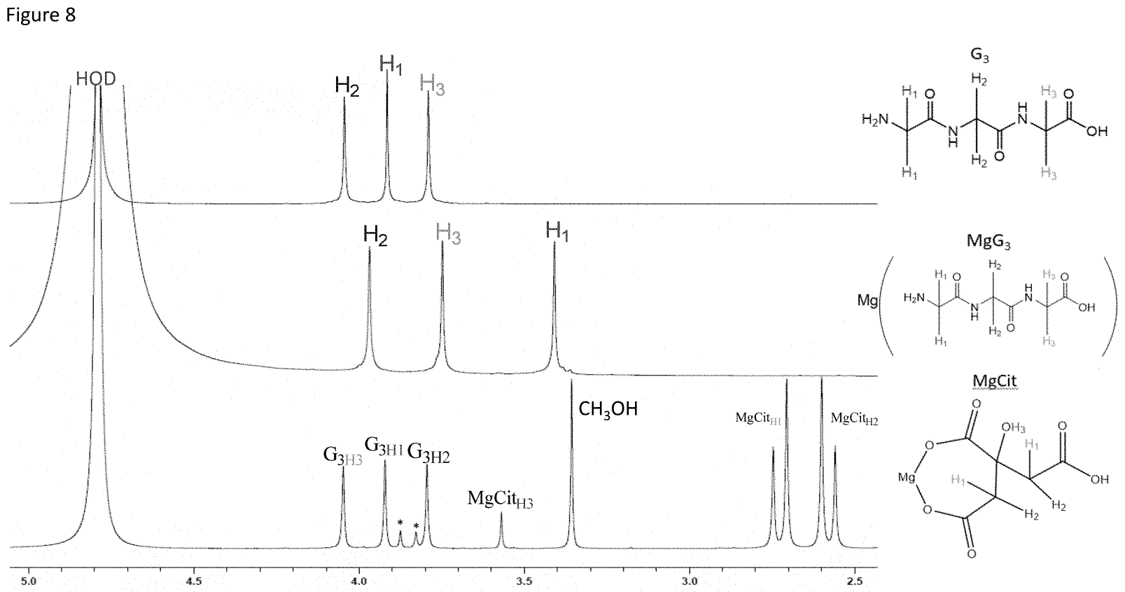

FIG. 9 depicts the characterization of G3 via ¹³C NMR. NMR taken in D₂O.

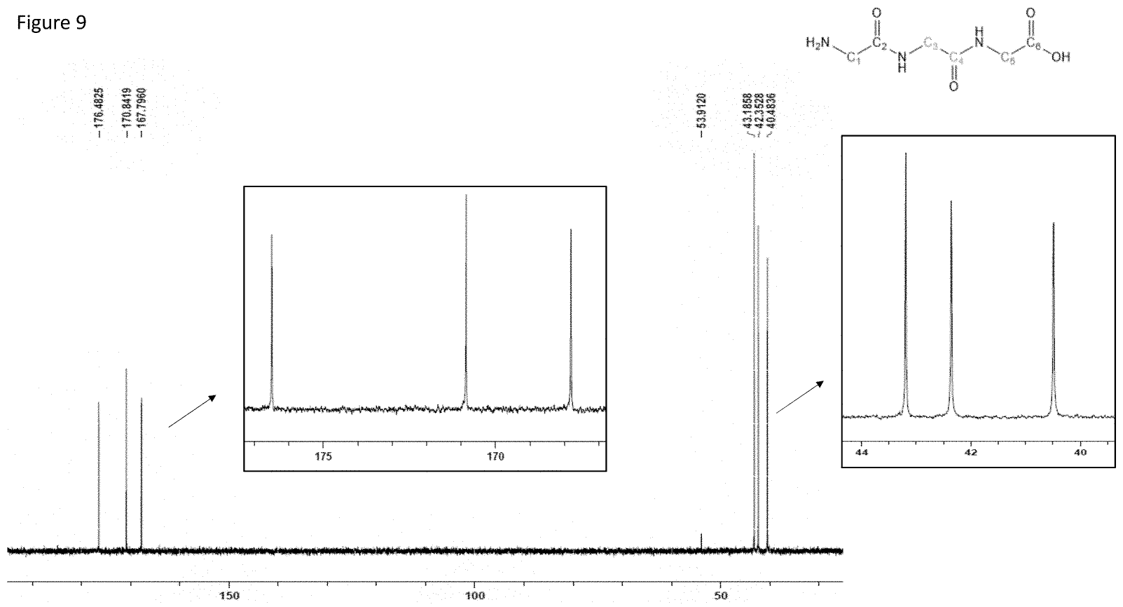

FIG. 10 depicts the characterization of Mg(G3) via ¹³C NMR (2 hrs). NMR taken in D₂O.

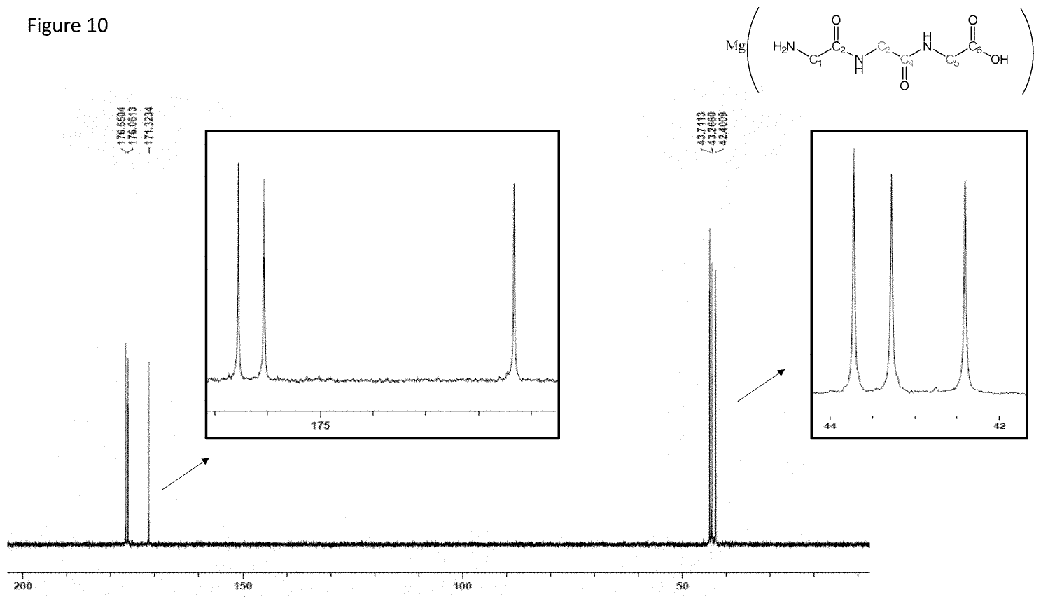

FIG. 11 depicts the ¹³C NMR overlay of G3 and Mg(G3). NMR taken in D₂O. Both G3 and Mg(G3) are expected to have six (6) carbon signals; both spectra exhibit six carbon signals. The ¹³C NMR shows a distinct downfield shift of the carbons in the region comprised of carboxylic acid carbons as well as amide carbons, thus suggesting chelation in these regions. The ¹³C signals in the alkane region have become more compact, with one carbon showing a downfield shift. Specific carbons have yet to be assigned.

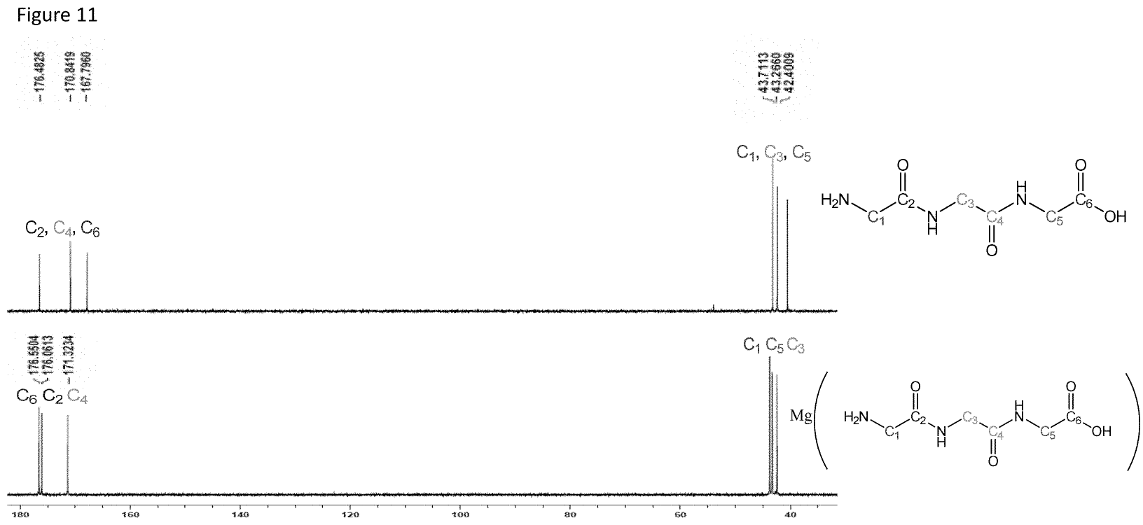

FIG. 12 depicts the characterization of G3 via HSQC NMR. Heteronuclear Single Quantum Coherence (HSQC)—determines coupling between single bond carbons and the corresponding protons within one bond distance. It was expected that each proton will only couple to one carbon environment—three signals means three couplings. Assignment of H₁ shows coupling to 40.64, indicating that this is C₁; Assignment of H₂ shows coupling to 42.41, indicating that this is C₃; Assignment of H₃ shows coupling to 43.21, indicating that this is C₅.

FIG. 13 depicts the characterization of G3 via HMBC NMR. Heteronuclear Multiple Bond Correlation (HMBC)—determines coupling between carbons two to three bonds away and the corresponding protons. There are three expectations: H₁ will have one coupling, H₂ will have two couplings, and H₃ will have two couplings. Assignment of H₁ shows coupling to 167.77, suggesting that this is C₂. Assignment of H₂ shows coupling to 167.77 and 170.79, suggesting that these are C₂ and C₄ respectively. Assignment of H₃ shows coupling to 170.79 and 176.36, suggesting that these are C₄ and C₆ respectively.

FIG. 14 depicts the characterization of Mg(G3) via HSQC NMR. Assignment of H₁ shows coupling to 43.51, suggesting that this is C₁. Assignment of H₂ shows coupling to 42.36, suggesting that this is C₃. Assignment of H₃ shows coupling to 43.14, suggesting that this is C₅.

FIG. 15 depicts the characterization of Mg(G3) via HMBC NMR. NMR taken in D₂O. Assignment of Hi shows coupling to 175.88, suggesting that this is C₂. Assignment of H₂ shows coupling to 171 and 175.8, suggesting that these are C₄ and C₂ respectively. Assignment of H3 shows coupling to 171 and 176.5, suggesting that these are C₄ and C₆ respectively.

FIG. 16 depicts characterization of G3 via ¹H NMR in DMSO. DMSO participates in hydrogen bonding with the carboxylic acid. Subsequently, this participation in binding results in unique environments of the H₃ protons nearest the carboxylic acid. These unequal environments result in splitting of the H₃ proton. Integration is 1, so it's still one proton.

FIG. 17 depicts the characterization of Mg(G3) via ¹H NMR in DMSO. Chelation of G3 to magnesium through the carboxylic acid moiety results in reestablished molecular symmetry, thus resulting in the disappearance of the H₃ proton splitting, further suggesting that this is absolutely the H₃ environment. Deprotonation of the carboxylic acid would also cause this occurrence.

FIG. 18 depicts the characterization of Mg(G3) via TGA and DSC. Overlay of G3 TGA, Mg(G3) TGA and DSC are bottom right of the figure. Mass percent change of 6.582% corresponds to the loss of one water/hydroxide from Mg(G3)(H₂O)₂(OH)—calculated to 6.42%. Hydroxide expected to be in lattice. Mass percent change of 14.09% corresponds to the loss of two waters from Mg(G3)(H₂O)₂—calculated to 14.5%. Graph features from 300° C. onward are solely from G3 decomposition as shown by corresponding G3 curve.

FIG. 19 depicts plots showing the effect of increasing citric acid in the reaction to produce a metal tri-amino acid chelate. Trace 1 has 0.01 equivalents of citric acid (reaction run for 2 hrs); Trace 2 has 0.1 equivalents of citric acid, Trace 3 has 0.25 equivalents of citric acid, Trace 4 is in citrate buffer (0.4 equivalents), Trace 5 is in citrate buffer (0.4 equivalents) for 24 hrs. Asterisks indicate the integrated citric acid peak(s). † indicates the integrated magnesium citrate peak(s).

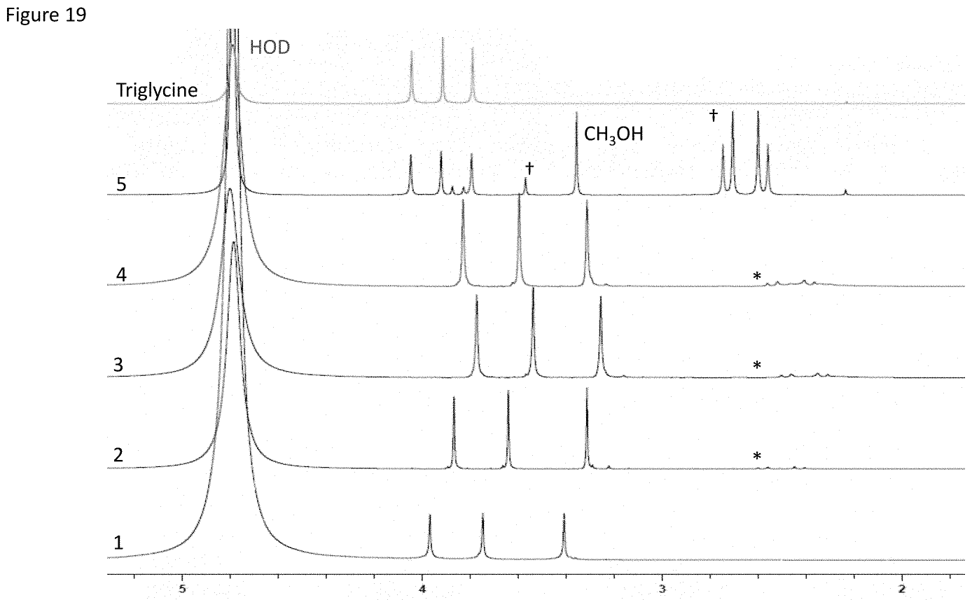

FIG. 20 depicts the characterization of Mg(G3) with 0.25 Meq of citric acid (CA) via ¹H NMR. NMR taken in D₂O. Asterisk indicates peaks due to ethanol in the lattice. Integral ratio of desired product peak to citric acid peak employed for yield determination and percent composition. Yield is roughly stoichiometric. Composition is 90% Mg(G3) (1.26 g) and 10% citric acid (140 mg).

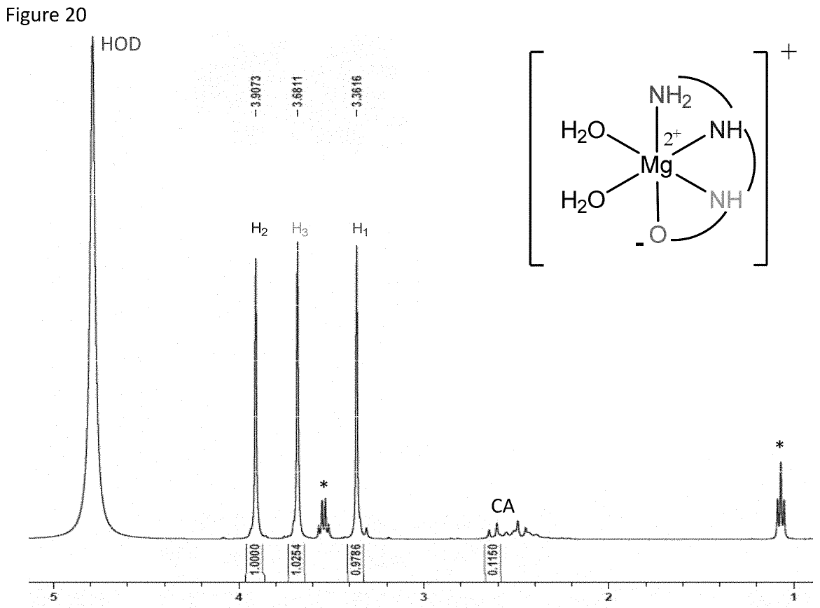

FIG. 21 depicts the characterization of Mg(G3) citrate buffer precipitate (magnesium citrate) via FT-IR. Use bottom IR for most accurate representation of MgCit.

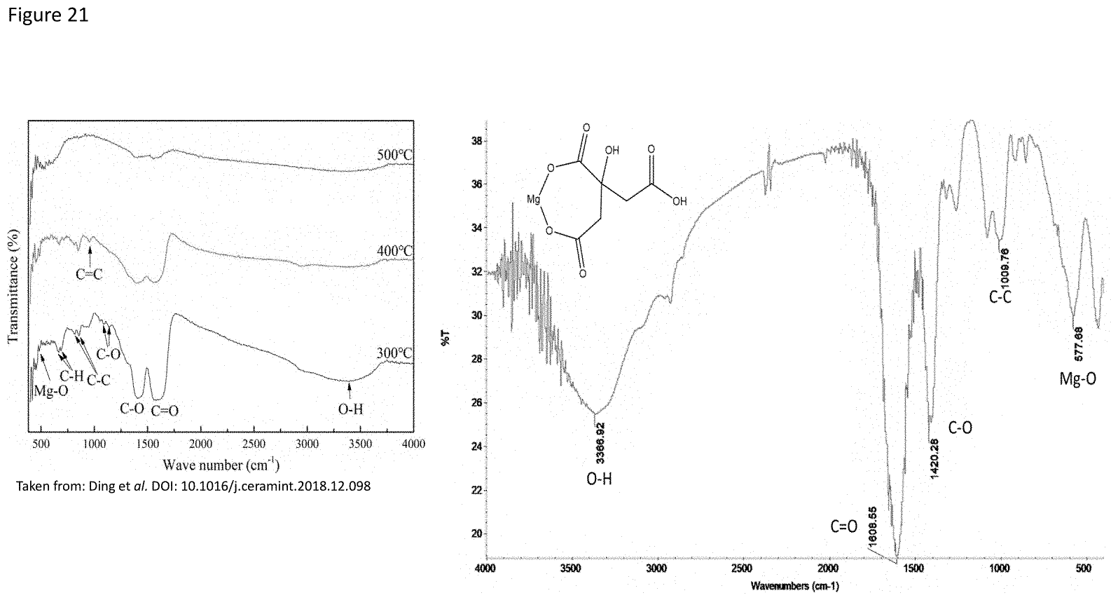

FIG. 22 depicts plots characterizing the stability of Mg(G3) in solution via ¹HNMR. The complex shows considerable stability for periods up to 72 hrs. At 72 hrs, an observable upfield shift occurs, which may be due to isomer formation. There is no observable change in proton integration. NMR taken in D₂O.

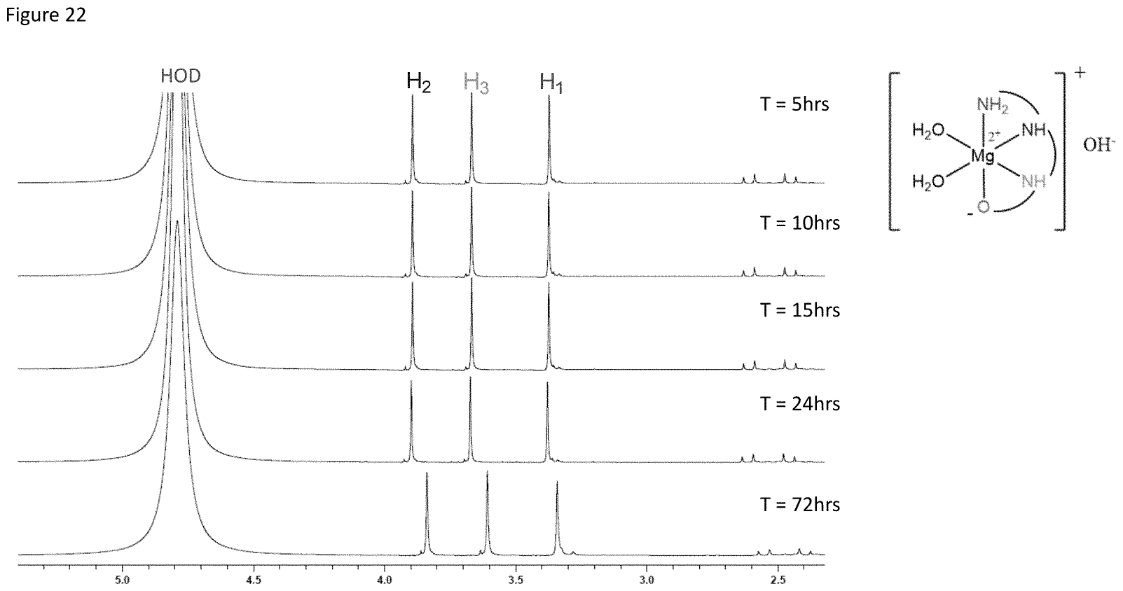

FIG. 23 depicts plots characterizing the stability of Mg(G3) in solution at 4° C. via ¹H NMR. At 4° C., there is observable splitting of all proton environments. This splitting is indicative of kinetic isomers that result from different binding modes of the G3 ligand—this further suggests coordination at all Lewis base positions given that all protons are impacted by this change in binding modes. When left to return to room temperature, the proton signals return to the recognizably stable Mg(G3) complex—which further suggests that isomers are due to slowed kinetics. NMR taken in D₂O.

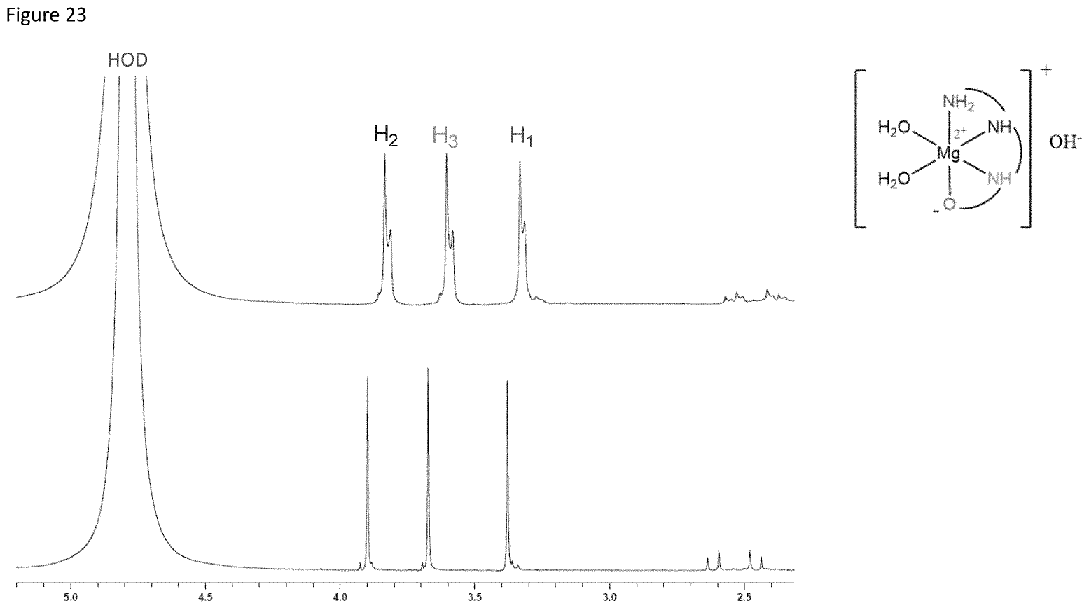

FIG. 24 depicts a graph illustrating the cellular uptake of Mg(G3) (green), MgBG (red), and MgCl₂ (blue) in CaCo-2 cells analyzed at 1 hr (left—with included inset), 4 hr (middle), and 24 hr (right). Both 1 and 4 hr time points show the significantly increased cellular uptake of Mg(G3) relative to MgBG and MgCl₂, with 24 hrs showing cell saturation.

FIG. 25 depicts a graph illustrating a kinetic evaluation of the cellular uptake of Mg(G3) (green), MgBG (red), and MgCl₂ (blue) in Caco-2 cells analyzed at 1 hr (left), 4 hr (middle), and 24 hr (right—inset included to show kinetic evaluation at points before concentrations that reach cell saturation). Kinetic ratios for Mg(G3), MgBG, and MgCl₂ are relatively conserved at 1 hr and 4 hr time points.

FIG. 26 depicts a method of making a metal tri-amino acid chelate. Specifically, FIG. 26 depicts a method of making a calcium triglycine chelate (1:1). A 1.0033 g sample of triglycine (G3—5.29 mmol; 1 eq.) was dissolved in approximately 10 mLs DI H₂O in a 50 mL round-bottom flask, with constant heating and stirring at 60° C. A separate solution of 587.9 mgs calcium chloride (CaCl₂—5.29 mmol; 1 eq.) was taken up in approximately 10 mLs DI H₂O, constantly stirred and heated to 60° C. The CaCl₂ solution was added to the triglycine solution—upon addition, the combined solution was colorless. The reaction was left to run for 1 hr at 60° C. The reaction was cooled to room temperature and filtered through a Buchner funnel (no solid was observed on the filter paper). The pH of the solution was found to be 6.02. The solution was concentrated down to approximately 3 mLs and the solid was precipitated with ethanol. Centrifugation was employed to pellet the solid, and the ethanol was decanted off. The solid was washed with diethyl ether to remove the ethanol. The sample was then recentrifuged, the ether decanted off, and the solid dried in vacuo overnight. The dried material was collected and massed. Yield was found to be nearly stoichiometric.

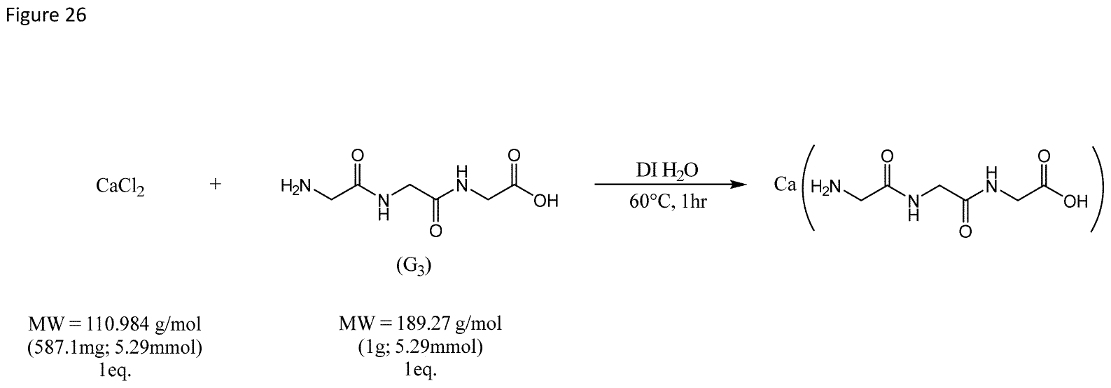

FIG. 27 depicts the characterization CaG3 via ESI-MS. The plot in the lower right hand side illustrates the isotopic distribution pattern for CaG3. The ESI-MS of CaG3 was taken in MeOH/TFA. The mass at 228 mz is indicative of [CaG3]+. Mass at 417 m/z is indicative of [Ca(G3)2]+. Other notable masses include the free G3 ligand and the subsequent sodium adduct at 190 m/z and 212 m/z respectively.

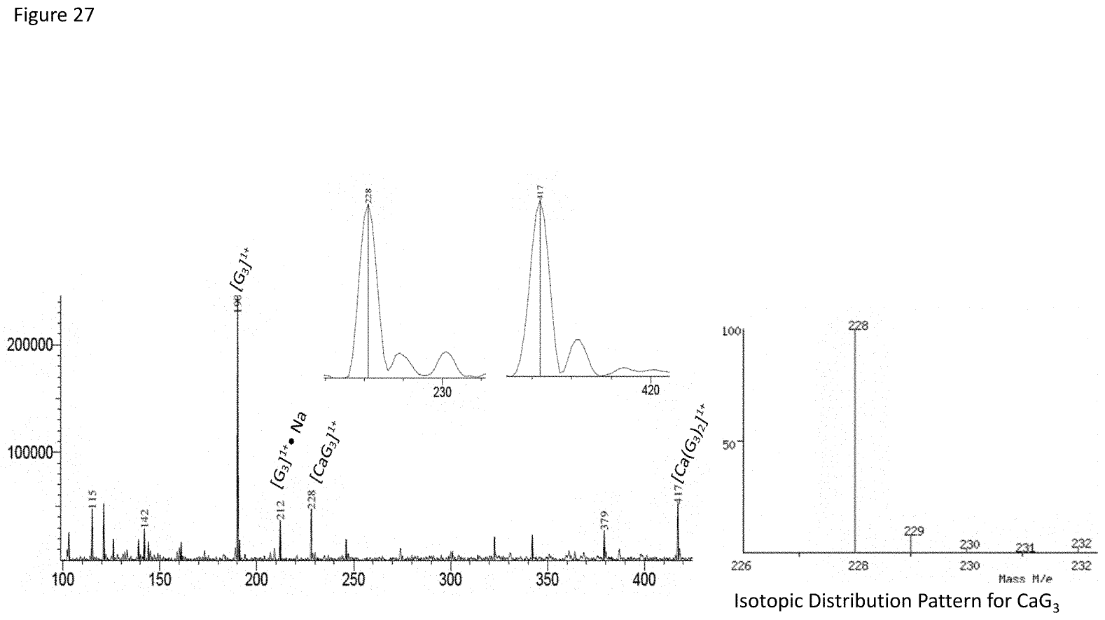

FIG. 28 depicts the characterization of triglycine (G3) via ¹H NMR. The NMR was taken in H₂O/D₂O (14.3% D₂O; VTOT=700 μL). The expected integration was 6 (2:2:2), and the observed integration was 6 (2:2:2).

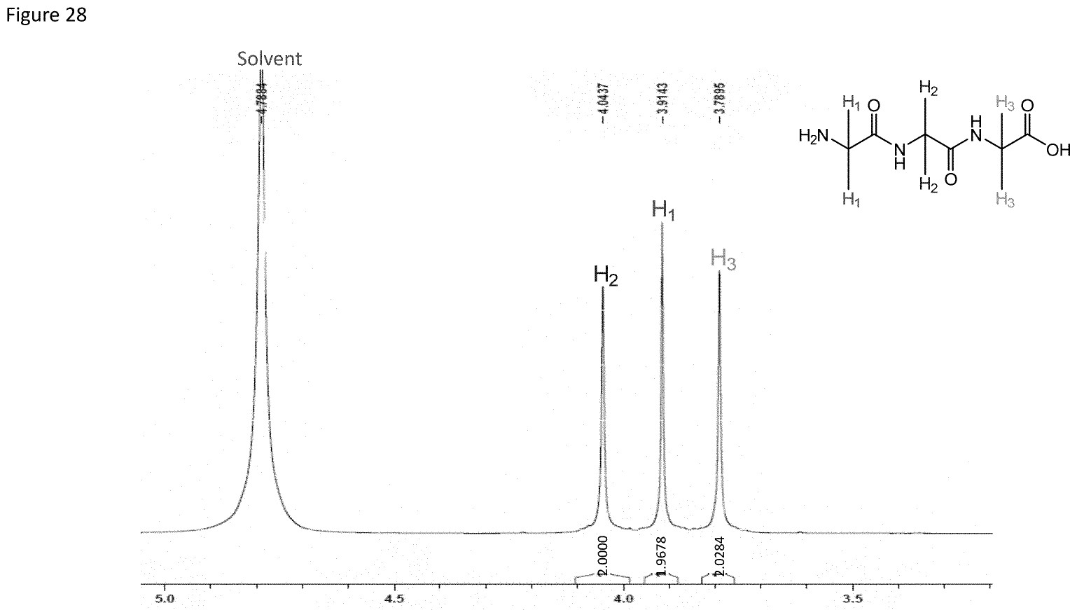

FIG. 29 depicts the characterization of CaG3 via ¹H NMR. NMR was taken in H₂O/D₂O (14.3% D₂O; VTOT=700 μL). The observed integration remains at 6. The observed splitting of proton signals in this analysis is not observed with stand-alone triglycine. Splitting is indicative of chelation.

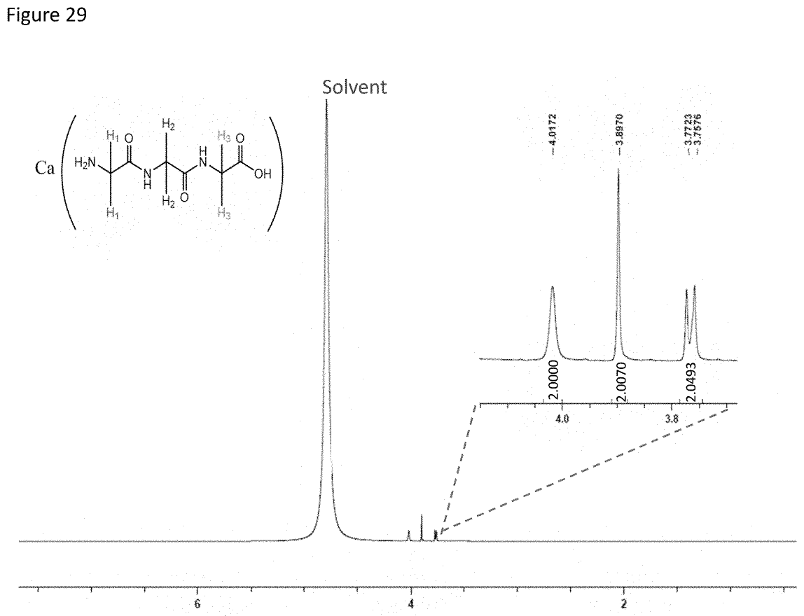

FIG. 30 depicts an ¹H NMR overlay of G3 and CaG3 (1 hr). This figure illustrates the splitting of the H₃ proton peak, and the upfield shift of proton peaks: H₁ Shift: 0.02 ppm; H₂ Shift: 0.03 ppm; and H₃ Shift: 0.02 ppm. The broadening of the H₂ proton peak suggests chelation at the nitrogen nearest the terminal acid—forming an entropically favored five-member ring structure similar to that of ZnG3. NMR was taken in H₂O/D₂O (14.3% D₂O; VTOT=700 μL).

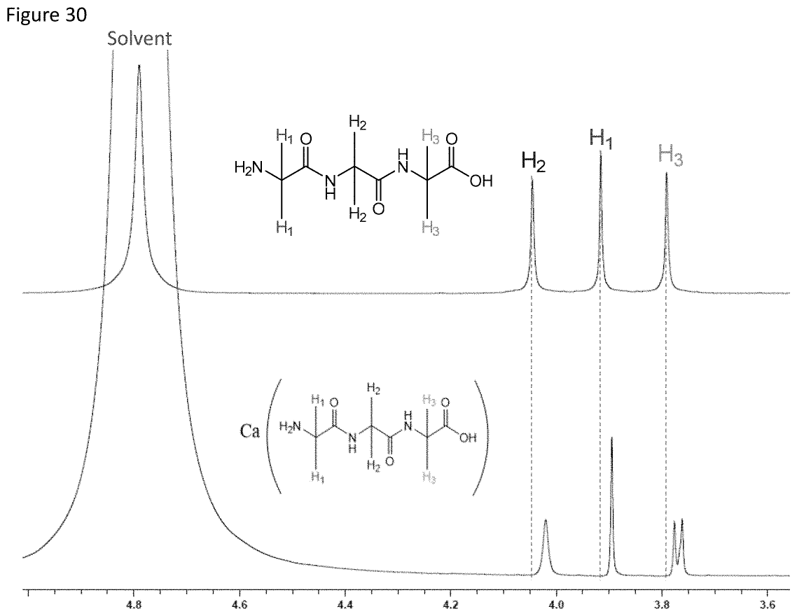

FIG. 31 depicts plots illustrating the confirmation of CaG3 reaction completion via ¹H NMR. NMR was taken in H₂O/D₂O (14.3% D₂O; VTOT=700 μL). The first observable change between G3 and CaG3 proton NMR occurs after one hour of reaction. There is no change in the CaG3 proton NMR after 1 hr, and there is no observable change in proton signal integration.

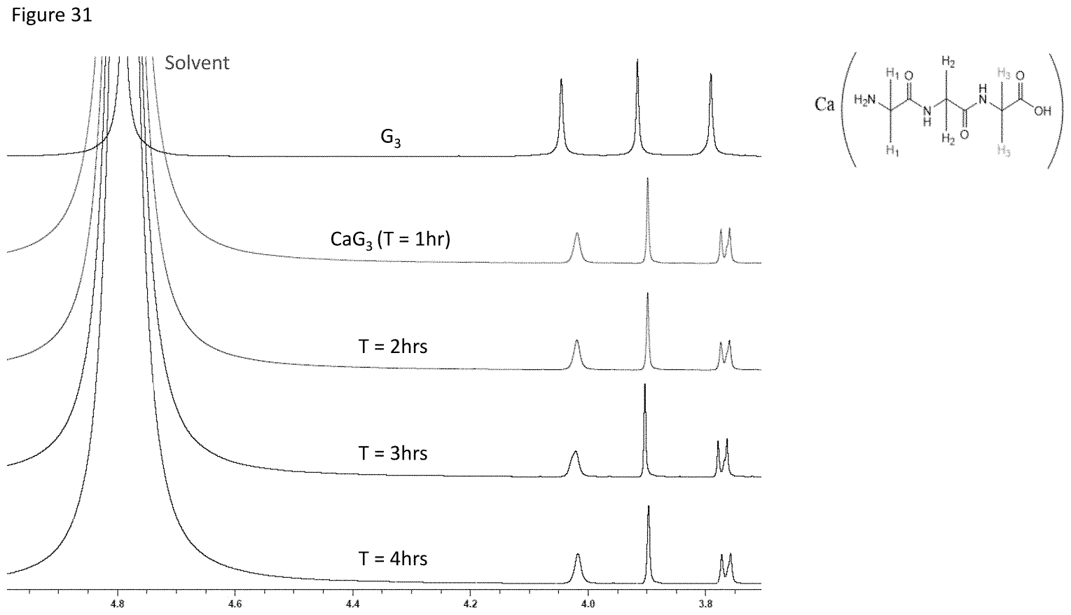

FIG. 32 depicts plots showing the characterization of CaG3 via ¹³C NMR. NMR was taken in H₂O/D₂O (14.3% D₂O; VTOT=700 μL). Changes in carbon signal intensity are visible, but there was no significant shifting of carbon signals.

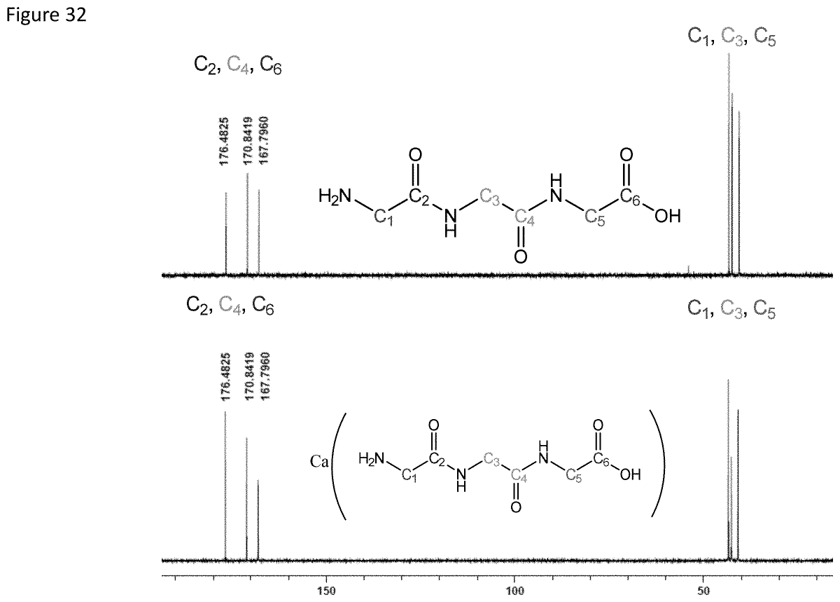

FIG. 33 depicts the characterization of CaG3 via HSQC NMR. NMR was taken in H₂O/D₂O (14.3% D₂O; VTOT=700 μL). The H₂ proton shows coupling to 42.555 ppm, suggesting that this is C₃. The H₁ proton shows coupling to 40.774 ppm, suggesting that this is C₁. The H₃ proton shows coupling to 43.333, suggesting that this is C₅.

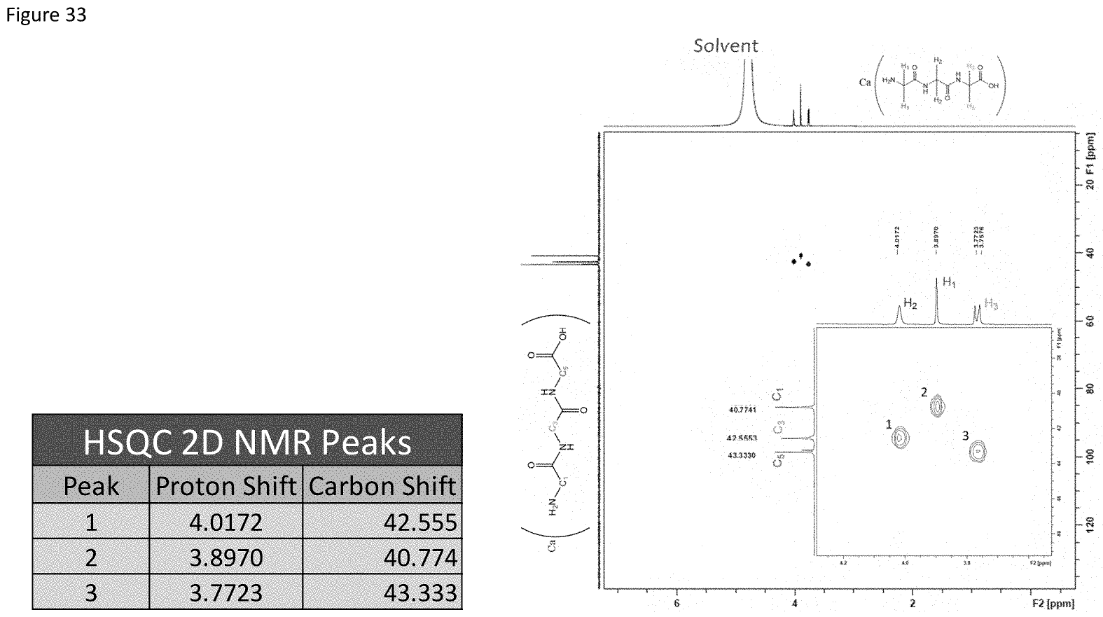

FIG. 34 depicts the characterization of CaG3 via HMBC NMR. NMR was taken in H₂O/D₂O (14.3% D₂O; VTOT=700 μL). There are two couplings expected for both H₂ and H₃; the overlapping point at 171.051 is believed to be C₄. The overlapping point between H₁ and H₂ at 168.009 is believed to be C₂. This leaves the signal at 176.709 to be C₆.

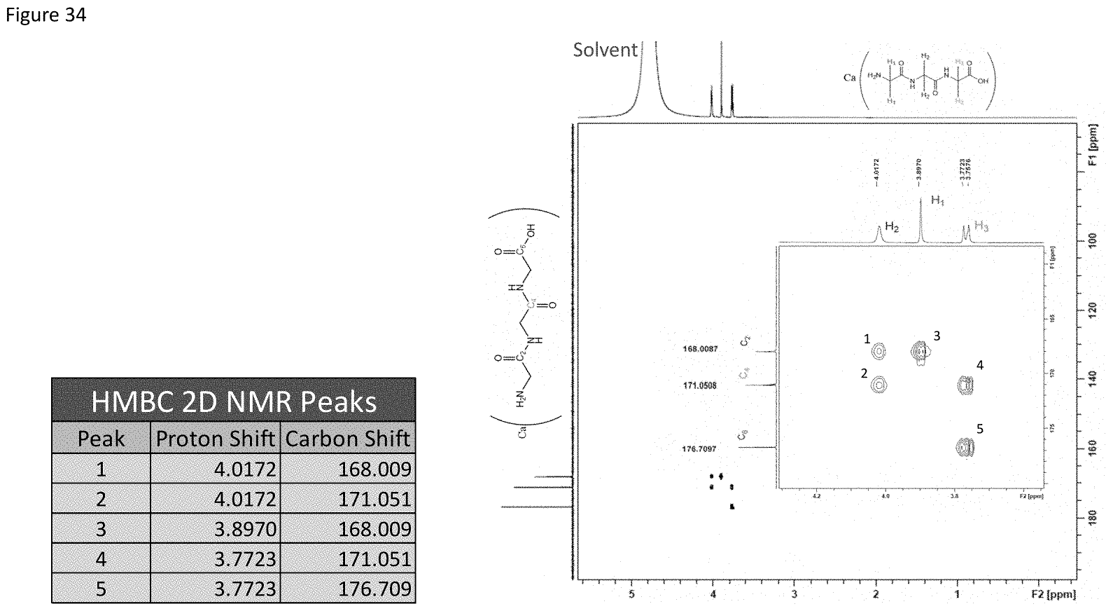

FIG. 35 depicts the characterization of CaG3 via elemental analysis. Elemental analysis suggests a calcium triglycine triaquo complex. Chloride is believed to be the anion present for charge balance. The presence of the chloride is supported by the elemental analysis data. Elemental coupled with NMR and IR suggest an octahedral CaG3 complex coordinated through the carboxylic acid and adjacent nitrogen of the G3 ligand (similar to that of ZnG3).

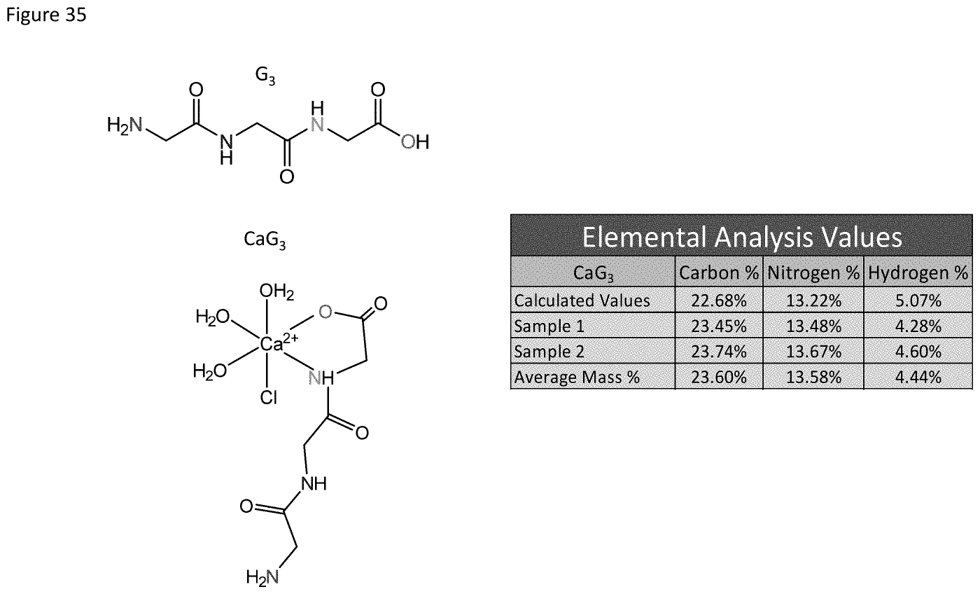

FIG. 36 depicts the characterization of CaG3 via TGA and DSC. The predicted chemical formula is Ca(G3)(H₂O)₃Cl (MW=317.7 g/mol). The first change of 11.99% corresponds to the loss of two waters from the complex (predicted to be 11.33%). The second mass change of 5.449% corresponds to the loss of a third water (predicted to be 6.40%). Further events are attributed to G3 decomposition.

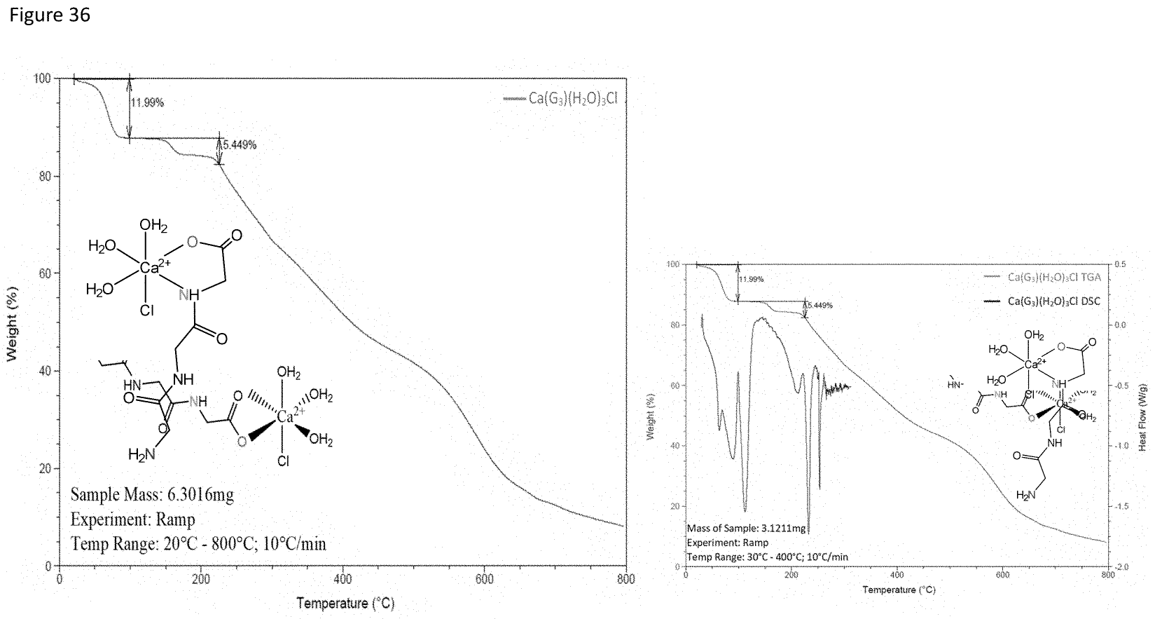

FIG. 37 depicts the synthesis of a CaG3 (1:2) chelate. Stated another way, FIG. 37 depicts the synthesis of Ca(G3)₂. A 1.0014 g sample of triglycine (G3—5.29 mmol; 1 eq.) was dissolved in approximately 10 mLs DI H₂O in a 50 mL round-bottom flask, with constant heating and stirring at 60° C. A separate solution of 294.6 mgs calcium chloride (CaCl₂—5.29 mmol; 1 eq.) was taken up in approximately 10 mLs DI H₂O, constantly stirred and heated to 60° C. The CaCl₂ solution was added to the triglycine solution—upon addition, the combined solution was colorless. The reaction was left to run for 1 hr at 60° C. The reaction was cooled to room temperature and filtered through a Buchner funnel (no solid was observed on the filter paper). The pH of the solution was found to be 6.77. The solution was concentrated down to approximately 3 mLs and the solid was precipitated with ethanol. Centrifugation was employed to pellet the solid, and the ethanol was decanted off. The solid was washed with diethyl ether to remove the ethanol. The sample was then recentrifuged, the ether decanted off, and the solid dried in vacuo overnight. The dried material was collected and massed. Yield was found to be nearly stoichiometric.

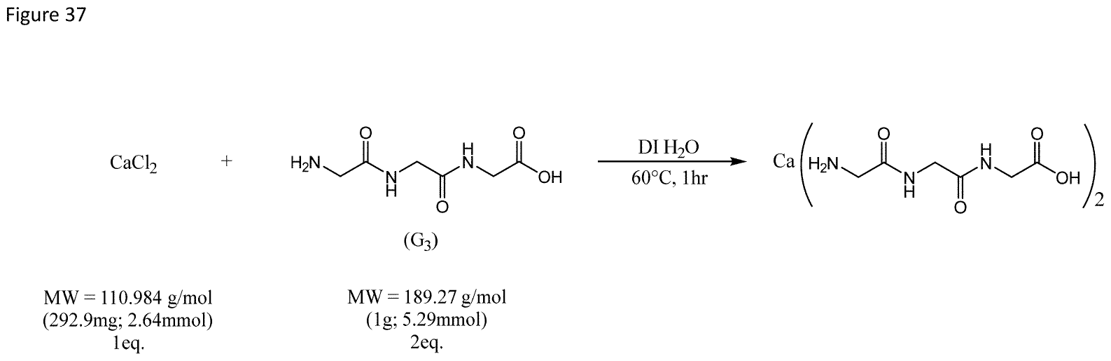

FIG. 38 depicts analysis of CaG3 (1:2) stoichiometry reaction via ESI-MS. ESI-MS of CaG3 was taken in MeOH/TFA. The mass at 228 m/z is indicative of [CaG3]+. The mass at 417 m/z is indicative of [Ca(G3)2]+. Other notable masses include the free G3 ligand and the Osubsequent sodium adduct at 190 m/z and 212 m/z respectively. The plot on the lower right hand side shows the isotopic distribution pattern for CaG3.

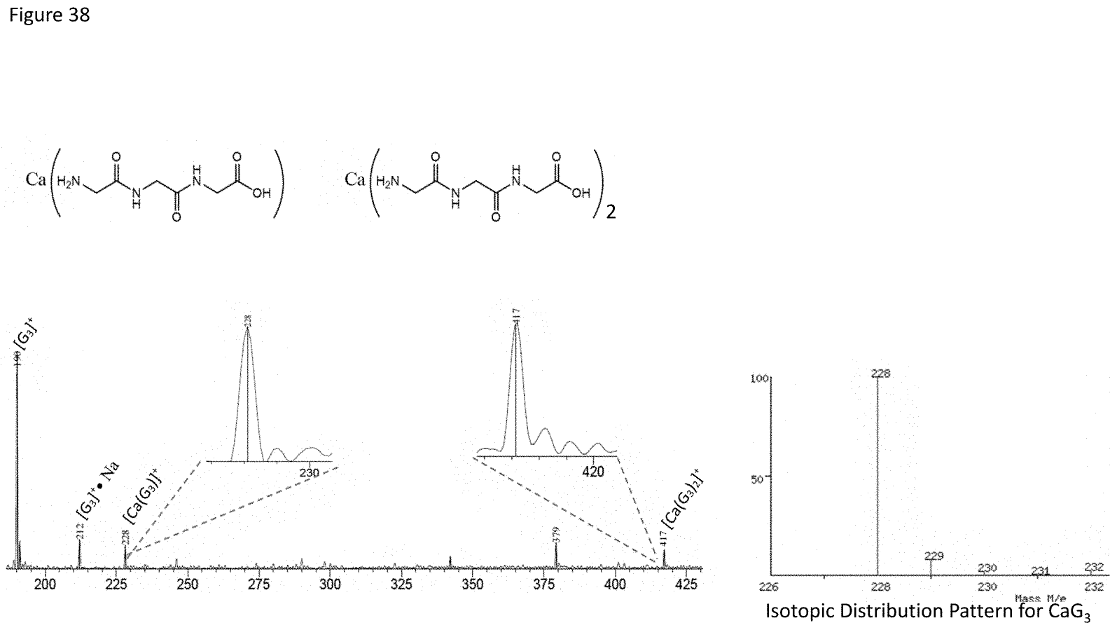

FIG. 39 depicts analysis of CaG3 (1:2) stoichiometry reaction via ¹H NMR. NMR was taken in H₂O/D₂O (14.3% D₂O; VTOT=700 μL). There was an expected integration of six (6) and an observed integration of six (6). There is the possibility of new proton signals given coordination modes affecting proton environments, however, no new proton signals were observed. ¹H NMR of CaG3 at a 1:2 Ca:G3 stoichiometry is similar to CaG3 at a 1:1 Ca:G3 stoichiometry.

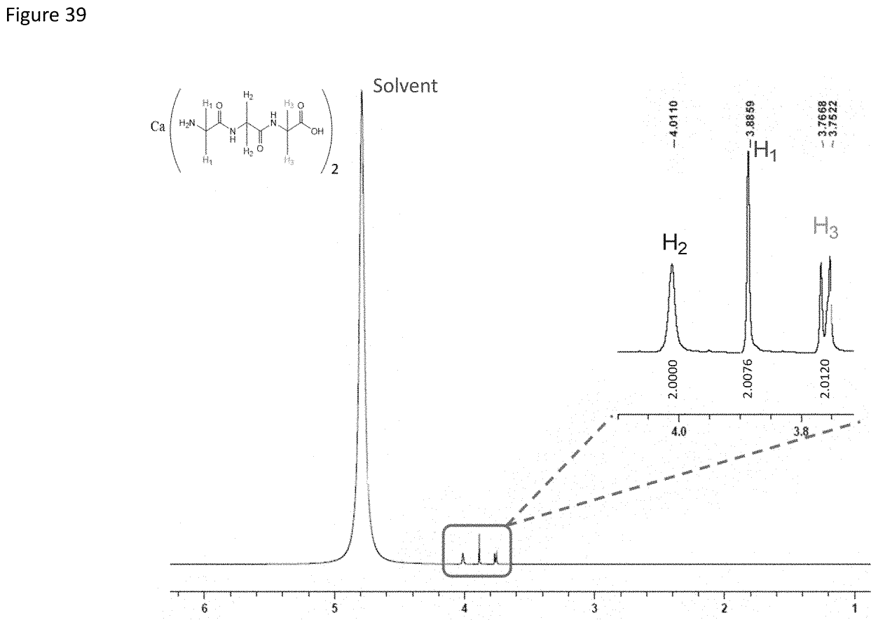

FIG. 40 depicts analysis of CaG3 (1:2) stoichiometry reaction via ¹H NMR. NMR was taken in H₂O/D₂O (14.3% D2O; VTOT=700 μL). CaG3 reaction conducted at a 1:2 Ca:G3 stoichiometry exhibits the same proton splitting pattern. Altering stoichiometry does cause a minor upfield shift in proton peak signals. Observed upfield peak shifting is most likely due to a change in pH brought about by changing stoichiometry. This type of upfield shift is common to alkali earth chelates and was also observed with increasing citric acid concentration in previously conducted Mg(G3) experiments. All NMR spectra are at a 1 hr time point.

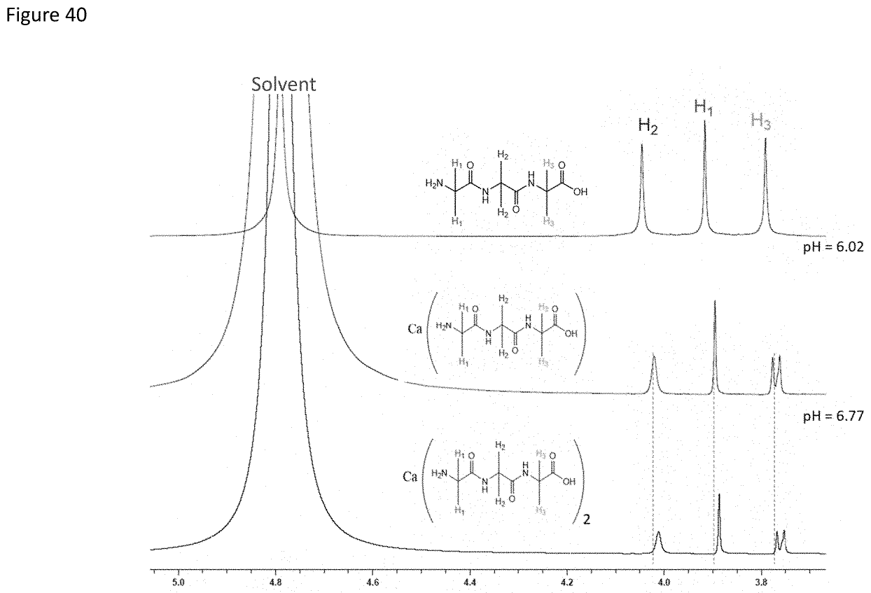

FIG. 41 depicts the characterization of CaG3 (1:2) via elemental analysis. Elemental analysis suggests a calcium diaquo bistriglycine chloride complex. Chloride is the anion present for charge balance. Presence of the chloride is supported by the elemental analysis data. Elemental coupled with NMR and TGA/DSC suggest an octahedral Ca(G3)2(H₂O)Cl complex coordinated through the carboxylic acid of the G3 ligand.

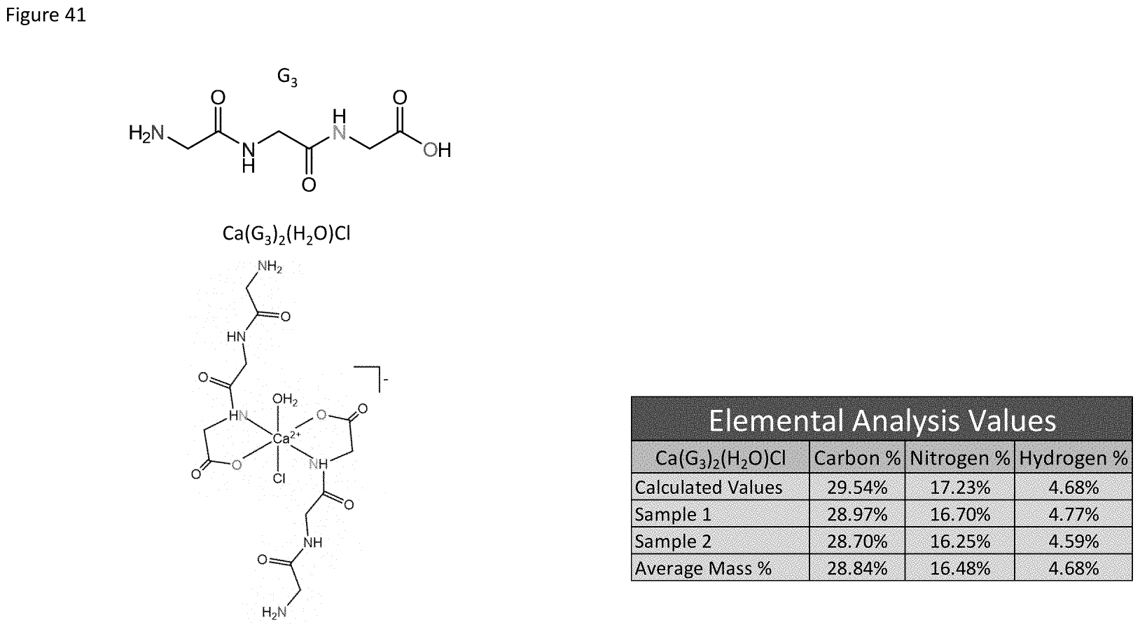

FIG. 42 depicts the characterization of Ca(G3)₂(H₂O)Cl via TGA and DSC. The predicted chemical formula is Ca(G3)2(H₂O)Cl (MW=470.1 g/mol). The first change of 3.117% corresponds to the loss of one water from the complex (predicted to be 3.82%. Further events are attributed to G3 decomposition.

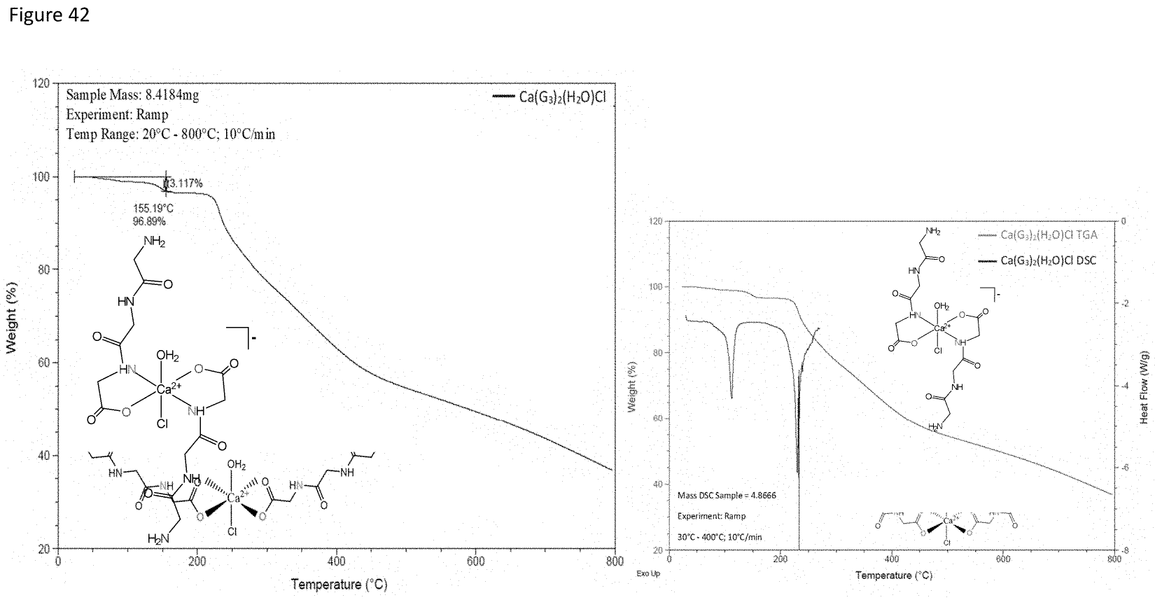

FIG. 43 depicts the synthesis of ZnG3 (1:1). A 1.0007 g sample of triglycine (G3—5.29 mmol; 1 eq.) was dissolved in approximately 10 mLs DI H₂O in a 50 mL round-bottom flask, with constant heating and stirring at 60° C. A separate solution of 725.8 mgs zinc chloride (ZnCl₂—5.29 mmol; 1 eq.) was taken up in approximately 10 mLs DI H₂O, constantly stirred and heated to 60° C. The ZnCl₂ solution was added to the triglycine solution—upon addition, the combined solution was colorless. The reaction was left to run for 1 hr at 60° C. The reaction was cooled to room temperature and filtered through a Buchner funnel (no solid was observed on the filter paper). The pH of the solution was found to be 4.9. The solution was concentrated down to approximately 3 mLs via rotary evaporation and the solid was precipitated out with isopropanol. Centrifugation was employed to pellet the solid, and the isopropanol was decanted off. The solid was washed with diethyl ether to remove the isopropanol. The sample was then recentrifuged, the ether decanted off, and the solid dried in vacuo overnight. The dried material was collected and massed. Yield was found to be nearly stoichiometric.

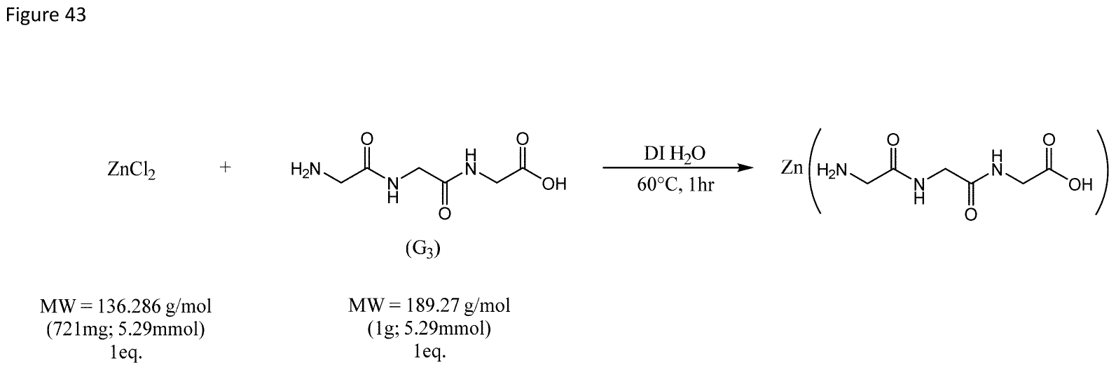

FIG. 44 depicts the characterization of ZnG3 via ESI-MS. ES I-MS of ZnG3 was taken in MeOH/TFA. Plot on the lower right indicates the isotopic distribution pattern for ZnG3. The mass at 252 m/z is indicative of [ZnG3]+, the mass at 288 m/z is indicative of [ZnG3]Cl+, and the negative trace was used to better illustrate the observed IDP (isotopic distribution pattern) which matches the predicted for this species (bottom right).

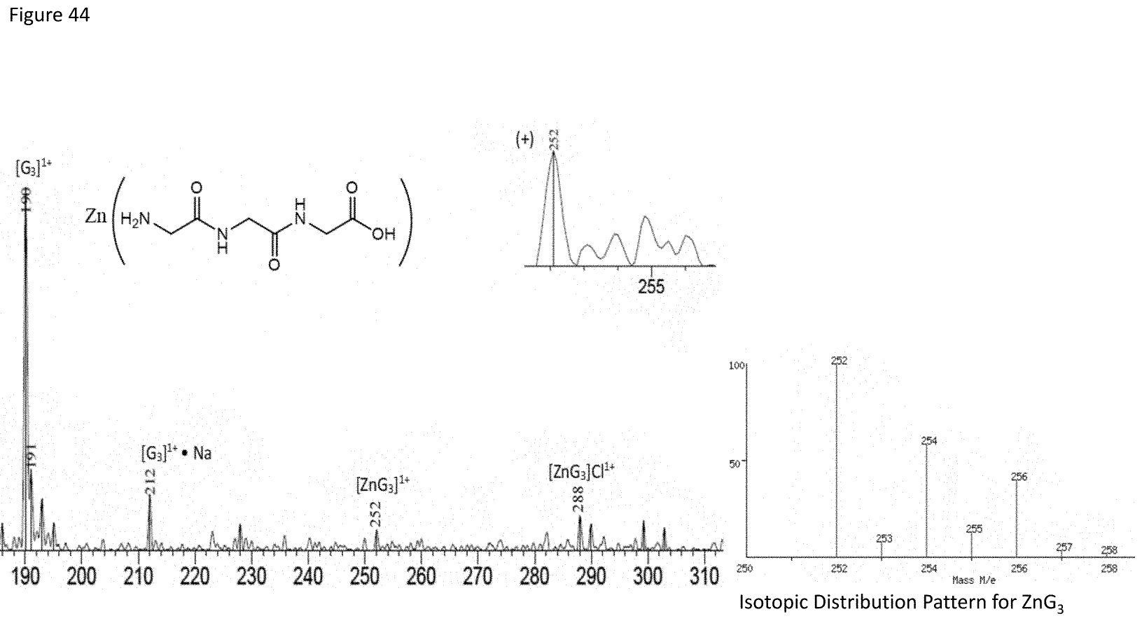

FIG. 45 depicts the characterization of ZnG3 via FT-IR. The change of CO stretch of the carboxylic acid from 991 cm-1 to 1028 cm-1, coupled with the disappearance of the carboxylic acid O—H stretch of G3, is indicative of chelation of the metal at the carboxylic acid moiety of G3.

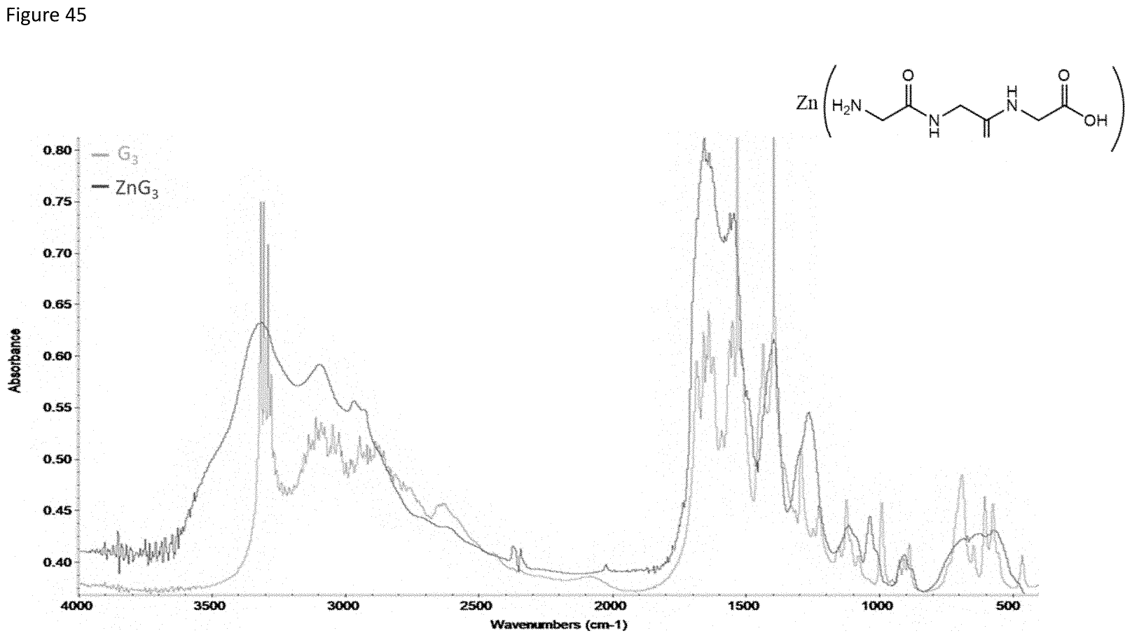

FIG. 46 depicts the characterization of ZnG3 via ¹H NMR. NMR was taken in H₂O/D₂O (14.3% D₂O; VTOT=700 μL). Observed integration remains at 6. Observed splitting of proton signals that is not observed with stand-alone triglycine. Splitting is indicative of chelation. Observed upfield shift of proton signals.

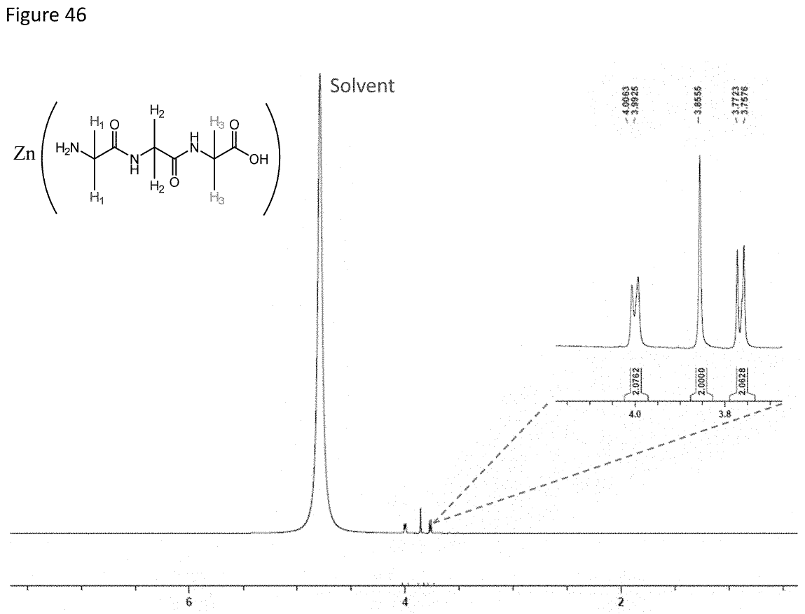

FIG. 47 depicts the ¹H NMR overlay of G3 and ZnG3 (1 hr). NMR was taken in H₂O/D₂O (14.3% D₂O; VTOT=700 μL). Splitting of proton peaks is observed, along with an upfield shift of the proton peaks.

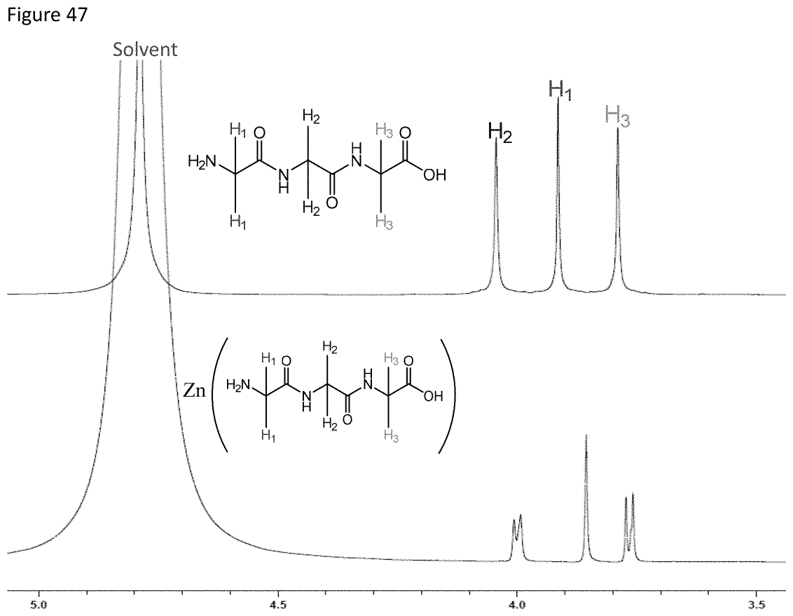

FIG. 48 depicts plots showing confirmation of reaction completion via ¹H NMR. NMR was taken in H₂O/D₂O (14.3% D₂O; VTOT=700 μL). First observable change between G3 and ZnG3 proton NMR occurs after reacting for one hour. There is no change in the ZnG3 proton NMR after 1 hr. Hour completion prompted reaction kinetic study for the first hour. There is no observable change in proton signal integration.

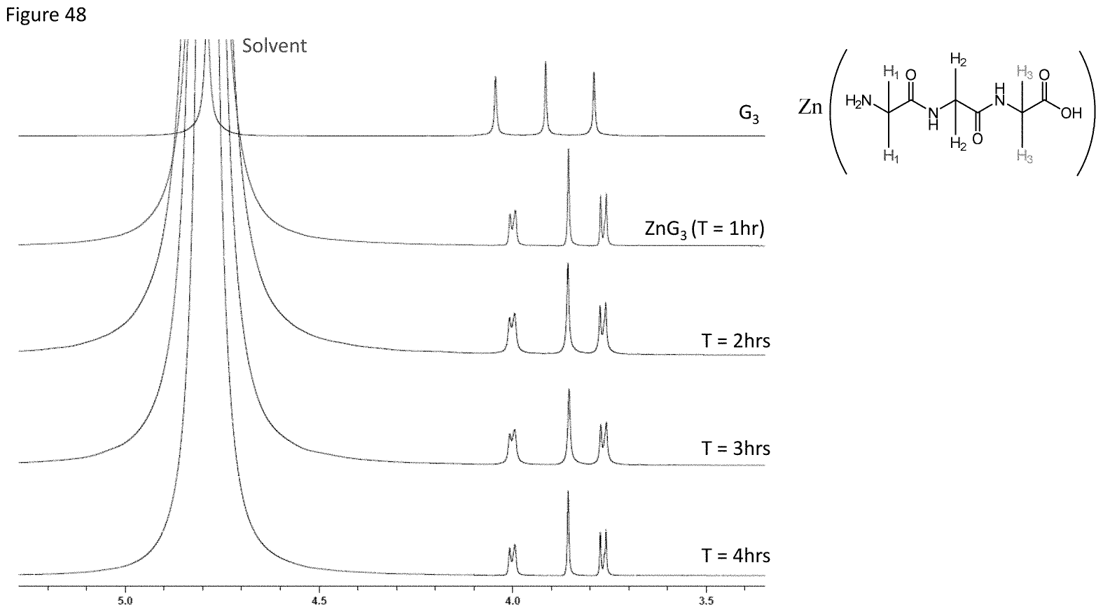

FIG. 49 depicts plots tracking the ZnG3 reaction for the first hour. NMR was taken in H₂O/D₂O (14.3% D₂O; VTOT=700 μL). No change in proton signal shift as indicated by ¹H NMR overlay of ZnG3 reaction after 10 min of reacting, suggesting reaction completion in approximately 10 min. The table in the lower right-hand corner shows proton shift values of ZnG3 at 10 min intervals.

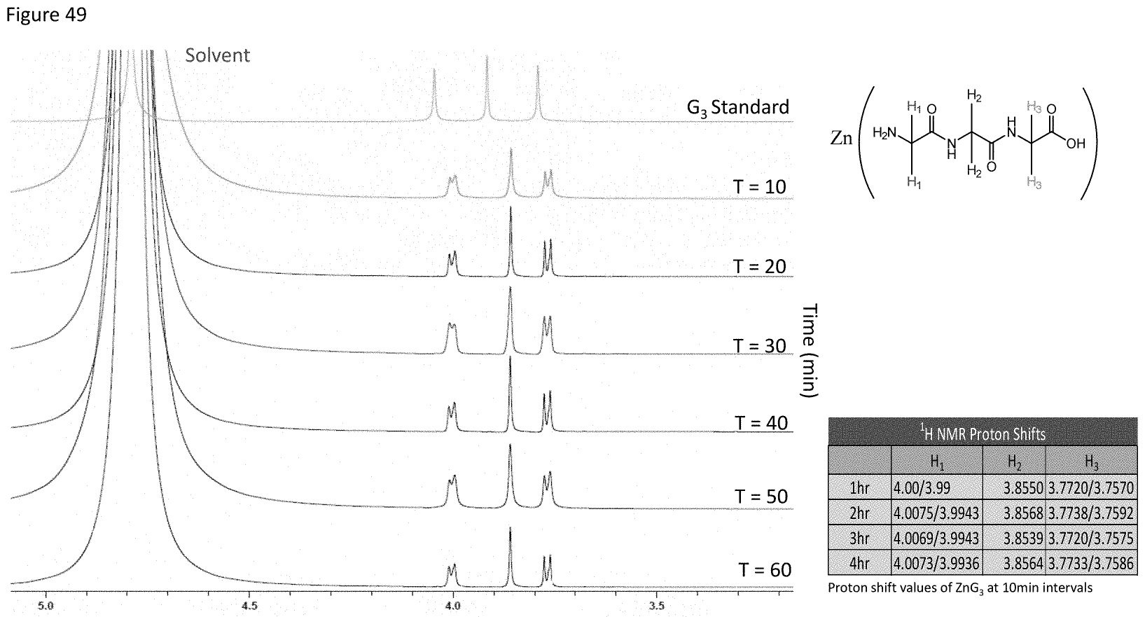

FIG. 50 depicts the characterization of ZnG3 via ¹³C NMR. NMR was taken in H₂O/D₂O (14.3% D₂O; VTOT=700 μL). Observable shift of most upfield carbon by 0.24 ppm.

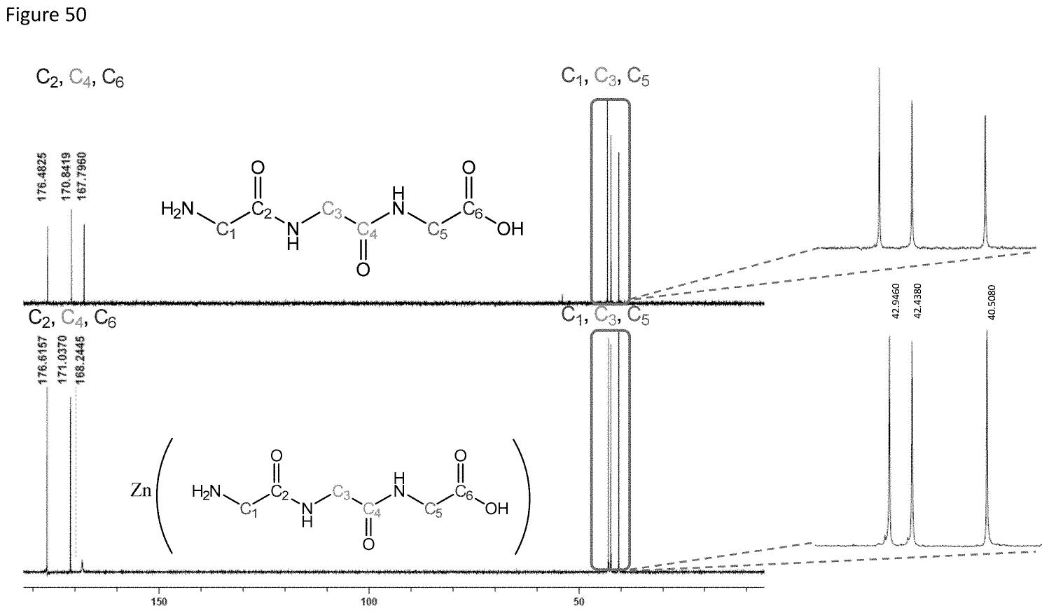

FIG. 51 depicts the characterization of G3 via HSQC NMR. NMR was taken in H₂O/D₂O (14.3% D₂O; VTOT=700 μL). Heteronuclear Single Quantum Coherence (HSQC)—determines coupling between sing bond carbons and the corresponding protons. It was expected that each proton will only couple to one carbon environment—three signals means three couplings. Assignment of H₁ shows coupling to 40.64, indicating that this is C₁. Assignment of H₂ shows coupling to 42.41, indicating that this is C₃. Assignment of H₃ shows coupling to 43.21, indicating that this is C₅.

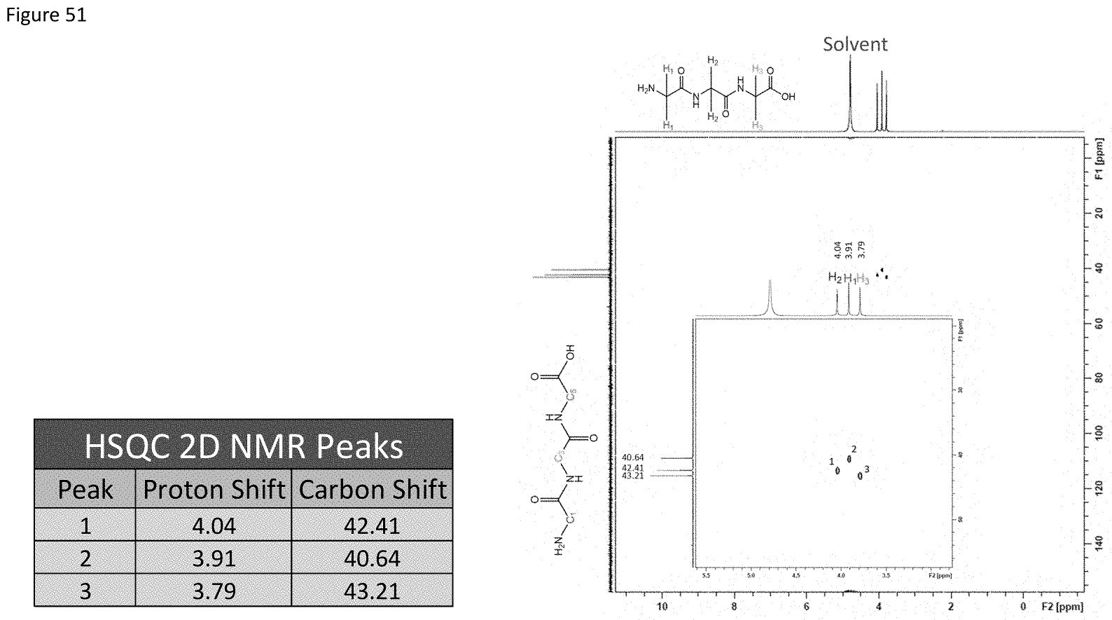

FIG. 52 depicts the characterization of G3 via Heteronuclear Multiple Bond Correlation (HMBC) NMR. NMR was taken in H₂O/D₂O (14.3% D₂O; VTOT=700 μL). HMBC determines coupling between carbons two to three bonds away and the corresponding protons. There were three expectations: H₁ will have one coupling, H₂ will have two couplings, and H₃ will have two couplings. Assignment of H₁ shows coupling to 167.77, suggesting that this is C₂. Assignment of H₂ shows coupling to 167.77 and 170.79, suggesting that these are C₂ and C₄, respectively. Assignment of H₃ shows coupling to 170.79 and 176.36, suggesting that these are C₄ and C₆ respectively.

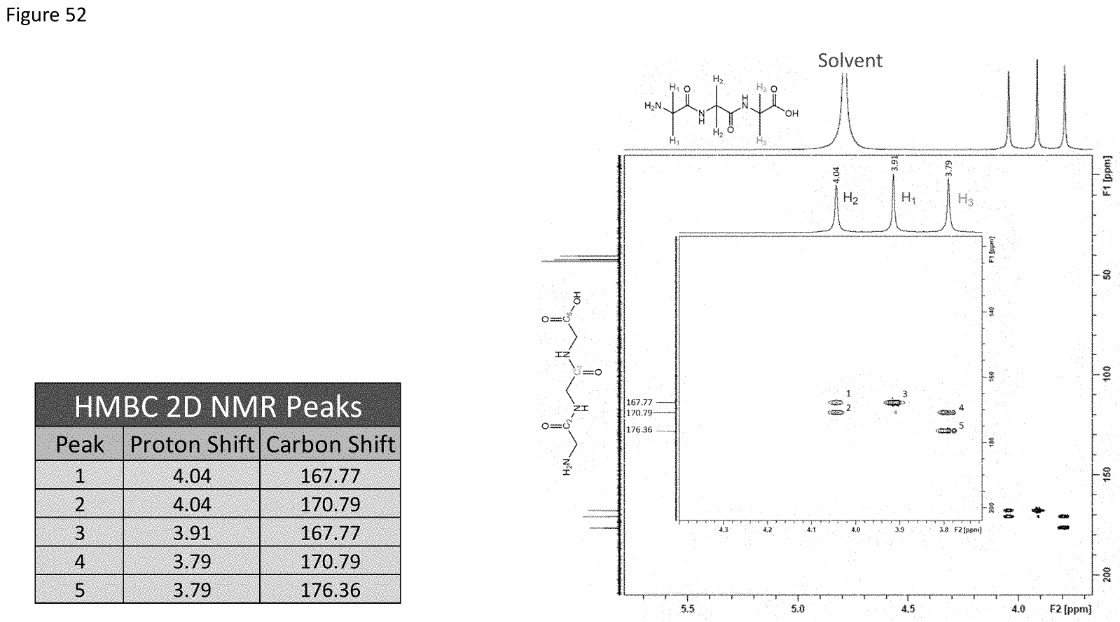

FIG. 53 depicts the characterization of ZnG3 via HSQC NMR. NMR was taken in H₂O/D₂O (14.3% D₂O; VTOT=700 μL). The H₂ proton shows coupling to 42.438 ppm, suggesting that this is C₃. The H₁ proton shows coupling to 40.508 ppm, suggesting that this is C₁. The H₃ proton shows coupling to 42.494, suggesting that this is C₅.

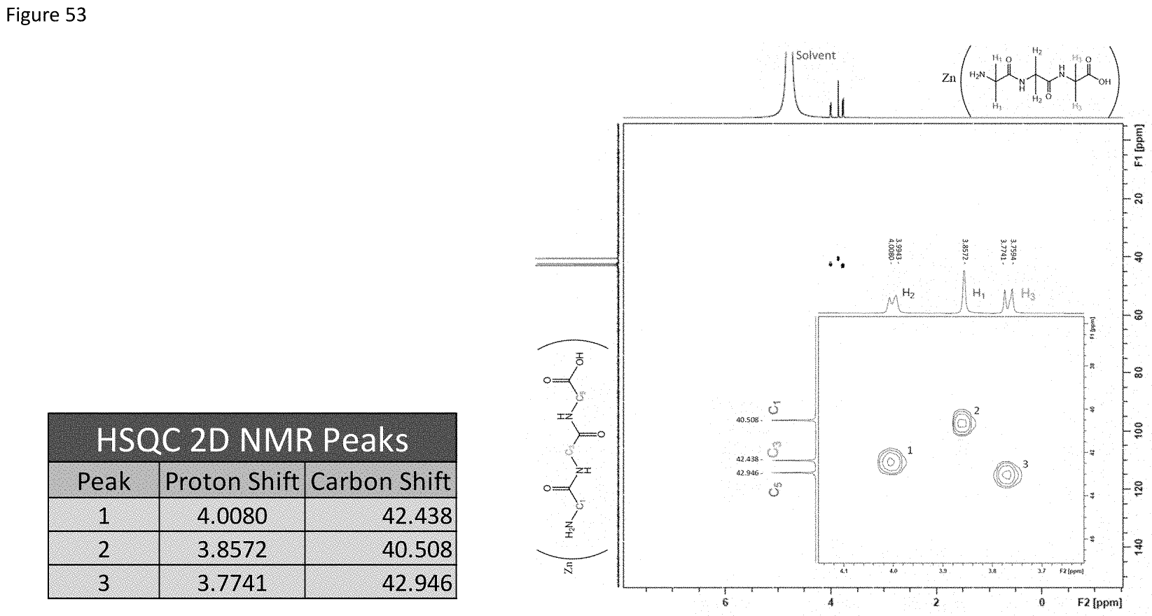

FIG. 54 depicts the characterization of ZnG3 via HMBC NMR. NMR was taken in H₂O/D₂O (14.3% D₂O; VTOT=700 μL). There are two couplings expected for both H₂ and H₃; the overlapping point at 171.143 is believed to be C₄. The overlapping point between H₁ and H₂ at 168.313 is believed to be C₂. This leaves the signal at 176.755 to be C₆. Combined HSQC and HMBC data suggest that the proton order for ZnG3 is the same as that of G3. Splitting suggests chelation near H₂ and H₃ protons—gives rise to a stable five-member ring structure—same as zinc glycinate.

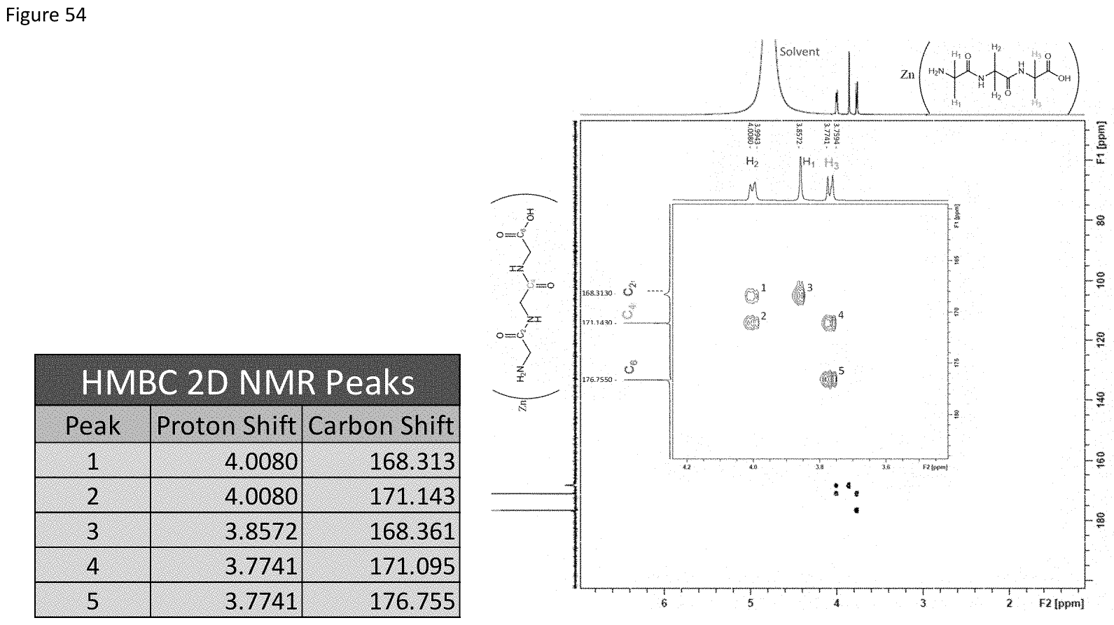

FIG. 55 depicts the characterization of ZnG3 via ¹H NMR in DMSO. NMR was taken in H₂O/DMSO (14.3% DMSO; VTOT=700 μL). There is no indication of DMSO interacting with the carboxylic acid proton (i.e. no observable splitting) supporting zinc is already coordinated at this region.

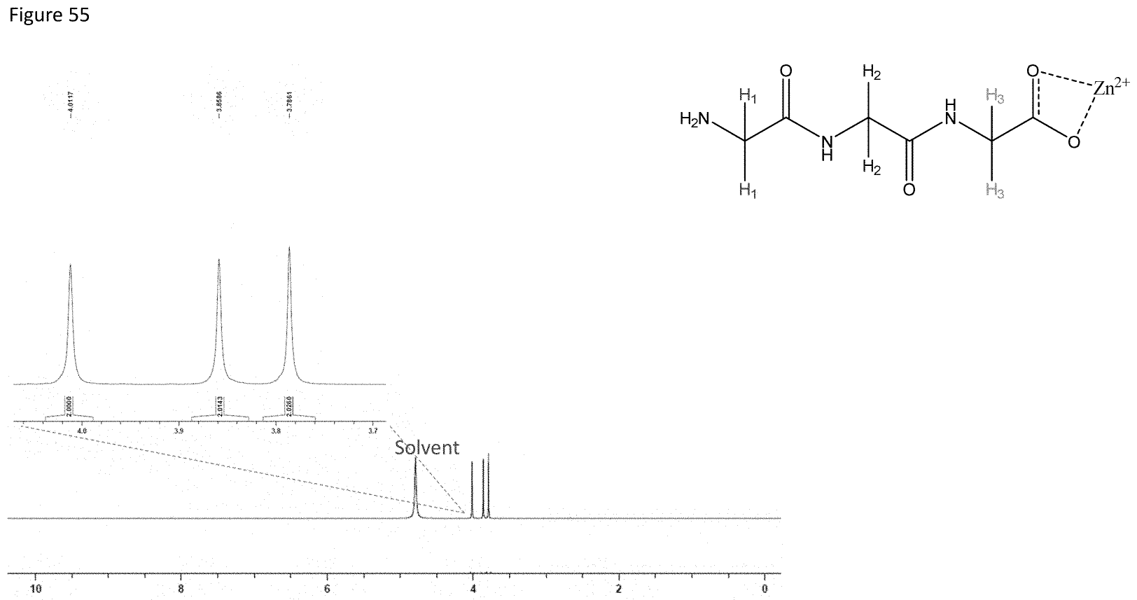

FIG. 56 depicts the characterization of ZnG3 via elemental analysis. Elemental analysis suggests a zinc triglycine diaquo complex. Chloride was believed to be the anion present for charge balance. Elemental coupled with NMR and IR suggest a tetrahedral diaqua ZnG3 complex with a five-membered ring formed through carboxylic acid and adjacent nitrogen of the G3 ligand.

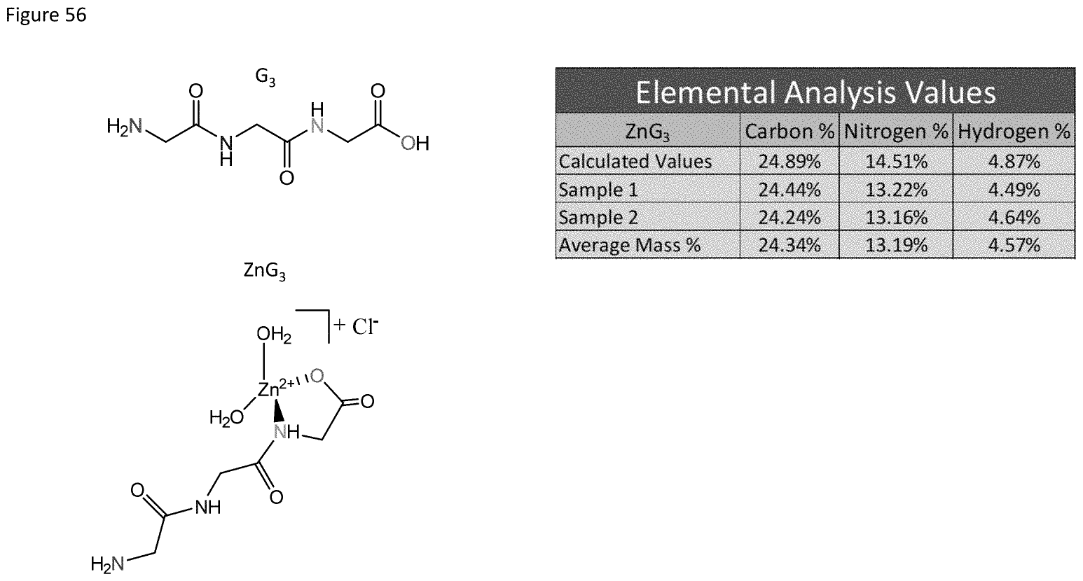

FIG. 57 depicts the characterization of ZnG3 via TGA and DSC. ZnG3 TGA was run on a 13.59 mg sample from 20° C.-800° C. at 10° C./min (left hand plot). ZnG3 DSC was run on a 7.498 mg sample from 30° C.-400° C. at 10° C./min (right hand plot). The predicted chemical formula is [Zn(G3)(H₂O)₂]Cl (MW=325.1 g/mol). The first change of 5.608% corresponds to the loss of one water from the complex (predicted to be 5.54%). The second mass change 5.169% corresponds to the loss of a second water (predicted to be 5.86%). Further events are attributed to G3 decomposition.

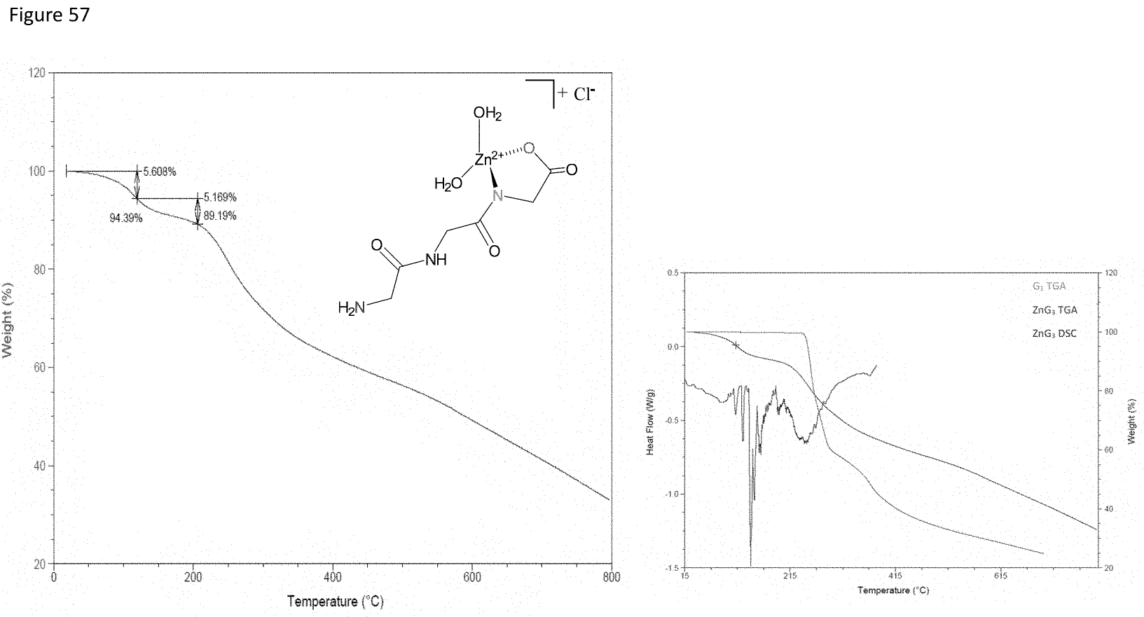

FIG. 58 depicts the cellular uptake of ZnG3. Assay was run on BioVision colorimetric zinc uptake assay kit. Absorbance was evaluated at 560 nm. Uptake was evaluated with HEK 293 kidney cells. It is observed that ZnG3 shows comparable cellular uptake to ZnCl₂.

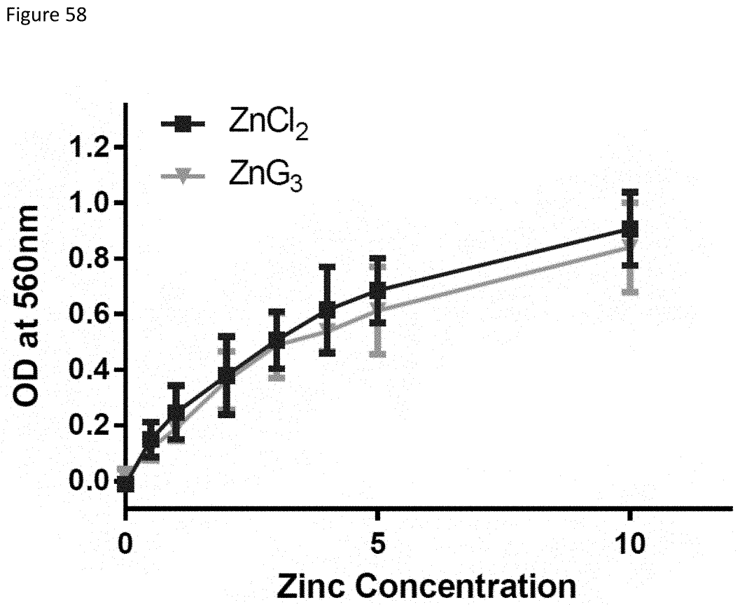

FIG. 59 depicts cellular uptake of ZnG3 vs. percent composition of zinc. Assay was run on BioVision colorimetric zinc uptake assay kit. Uptake was evaluated with HEK 293 kidney cells. Evaluation of cellular uptake relative to percent composition of zinc was evaluated. ZnCl₂ and ZnG3 have percent compositions of 47.97% and 22.58% respectively. It was expected that ZnG3 would have comparable cellular uptake to the salt ZnCl₂. As illustrated, ZnG3 drastically outcompetes ZnCl₂ in terms of cellular uptake relative to percent composition.

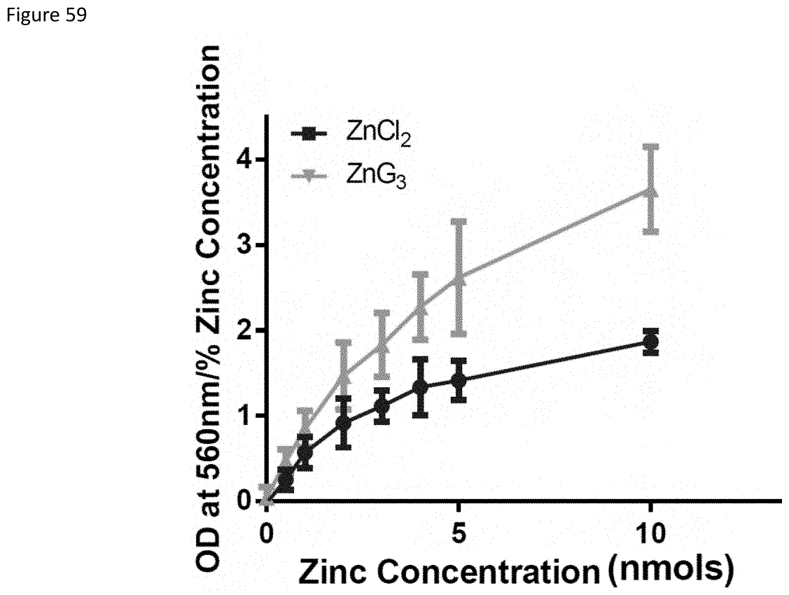

FIG. 60 details synthesis of a ZnG3 (1:2) chelate. A 1.0051 g sample of triglycine (G3—5.29 mmol; 1 eq.) was dissolved in approximately 10 mLs DI H₂O in a 50 mL round-bottom flask, with constant heating and stirring at 60° C. A separate solution of 361.6 mgs zinc chloride (ZnCl₂—2.64 mmol; 1 eq.) was taken up in approximately 10 mLs DI H₂O, constantly stirred and heated to 60° C. The ZnCl₂ solution was added to the triglycine solution—upon addition, the combined solution was colorless. The reaction was left to run for 1 hr at 60° C. The reaction was cooled to room temperature and filtered through a Buchner funnel (no solid was observed on the filter paper). The pH of the solution was found to be 5.84. The solution was concentrated down to approximately 3 mLs and the solid was crashed out with isopropanol. Centrifugation was employed to pellet the solid, and the isopropanol was decanted off. The solid was washed with diethyl ether to remove the isopropanol. The sample was then recentrifuged, the ether decanted off, and the solid dried in vacuo overnight. The dried material was collected and massed. Yield was found to be approximately stoichiometric.

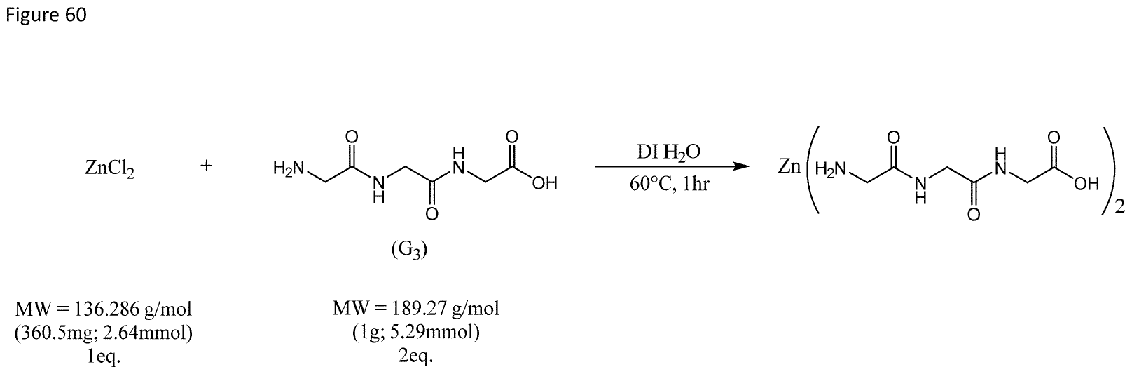

FIG. 61 depicts the analysis of ZnG3 (1:2) stoichiometry reaction via ¹H NMR. NMR was taken in H₂O/D₂O (14.3% D₂O; VTOT=700 μL). Expected integration of six (6), and there was an observed integration of six (6). There was the possibility of new proton signals given coordination modes affecting proton environments, however, no new proton signals were observed. ¹H NMR of ZnG3 at a 1:2 Zn:G3 stoichiometry similar to ZnG3 at a 1:1 Zn:G3 stoichiometry.

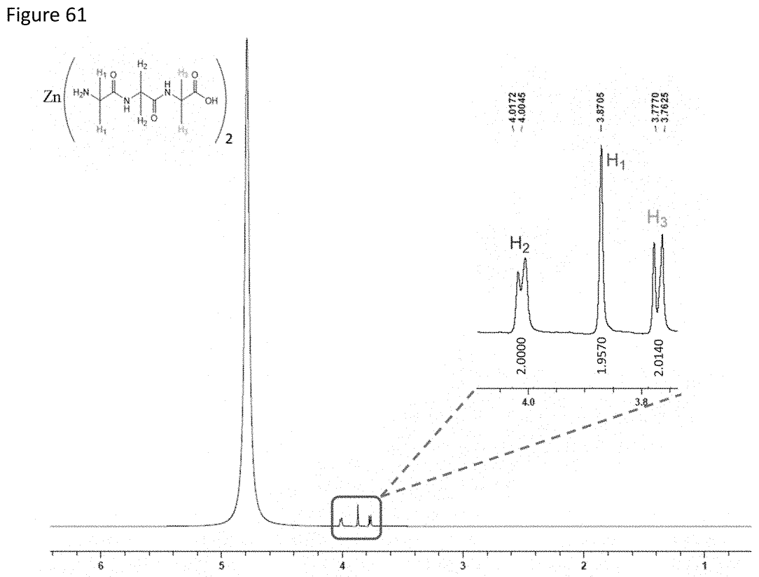

FIG. 62 depicts analysis of ZnG3 (1:2) stoichiometry reaction Via ¹H NMR. NMR was taken in H₂O/D₂O (14.3% D₂O; VTOT=700 μL). ZnG3 reaction conducted at a 1:2 Zn:G3 stoichiometry exhibits the same proton splitting pattern. Altering stoichiometry does cause a minor downfield shift in proton peak signals. Observed downfield peak shifting is most likely due to a change in pH brought about by changing stoichiometry. This type of downfield shift expected as zinc is electron dense and not electropositive like magnesium and calcium, causing a shift in the opposite direction. All NMR spectra are at a 1 hr time point.

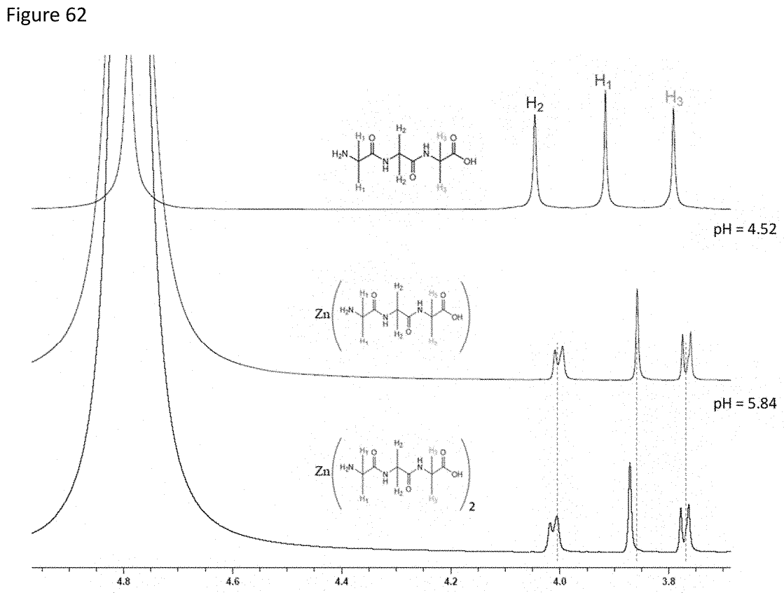

FIG. 63 depicts the characterization of ZnG3 (2:1) via elemental analysis. Elemental analysis suggests a zinc diaquo bistriglycine complex. Elemental analysis coupled with NMR and IR suggest an octahedral zinc diaquo bistriglycine complex with five-membered rings formed through the carboxylic acid and adjacent nitrogen of the G3 ligand.

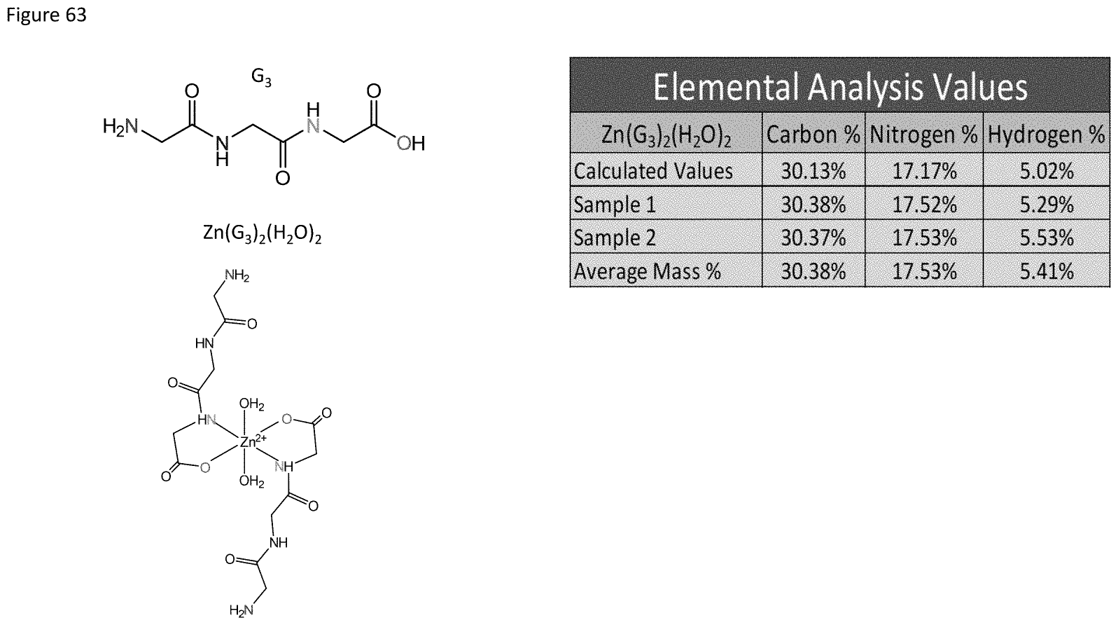

FIG. 64 depicts the characterization of ZnG3 via TGA and DSC. The predicted chemical formula is Zn(G3)₂(H₂O)₂ (MW=477.3 g/mol). The change of 14.07% would correspond to the loss of four waters—this could be attributed to lattice water. Further, high temperature events are attributed to G3 decomposition.

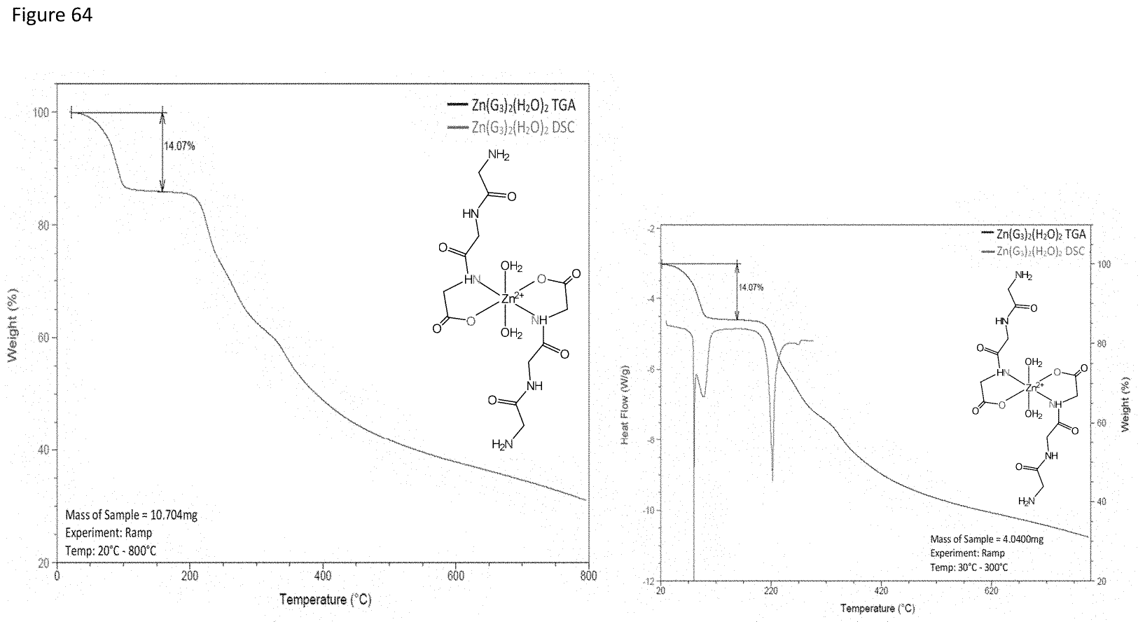

FIG. 65 depicts the characterization of MgG2 via ¹H NMR of both diglycine (G2—Left) and magnesium diglycine (MgG2—right). The ¹H NMR illustrate the change in electronic environment of the ligand protons that is consistent with magnesium coordination.

FIG. 66 depicts the characterization of MgG2 via ¹³C NMR of diglycine and MgDG illustrating a shift of observed carbon signals attributed to a change in electronic environment upon diglycine coordination to magnesium.

FIG. 67 depicts the characterization of MgG2 via 2D HSQC of both diglycine (left) and MgDG (right), which shows two correspondences, with each proton showing only one correspondence each to a singular carbon.

FIG. 68 depicts the characterization of MgG2 via HMBC of both diglycine (left) and MgG2 (right) with Hi showing only one correspondence and H₂ showing two correspondences.

FIG. 69 depicts the characterization of MgG2 via ESI-MS conducted in methanol.

FIG. 70 depicts the characterization of MgG2 via FT-IR of diglycine and MgDG conducted in a potassium bromide (KBr). Background CO₂ is observed at approximately 2350 cm-1.

FIG. 71 depicts the characterization of MgG2 via overlaid TGA/DSC of diglycine and MgDG. Both the TGA of diglycine (green) and MgDG (red) are provided, as well as the DSC of MgDG (blue).

FIG. 72 depicts graphs illustrating the cellular uptake of different chelates.

### DETAILED DESCRIPTION

The present disclosure provides compositions comprising metal di-amino acid or tri-amino acid chelate complexes, methods of using such compositions, and methods of making such compositions.

It is to be understood that this disclosure is not limited to the particular methods, compositions, or materials specified herein, but is extended to equivalents thereof as would be recognized by those ordinarily skilled in the relevant arts. It should also be understood that terminology employed herein is used for the purpose of describing particular embodiments only and is not intended to be limiting.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL” The endpoint may also be based on the variability allowed by an appropriate regulatory body, such as the FDA, USP, etc.

As used herein, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open ended term, like “comprising” or “including,” it is understood that direct support should be afforded also to “consisting essentially of” language as well as “consisting of” language as if stated explicitly and vice versa.

As used herein, the term “chelate” refers to a chemical compound in which a metal atom is attached to neighboring atoms of a di-amino acid or tri-amino acid ligand by at least two coordinate bonds. In some embodiments, a chelate of the present disclosure has three coordinate bonds with a ligand. In preferred embodiments, a chelate of the present disclosure has between two and six coordinate bonds between the metal and the di-amino acid or tri-amino acid ligand.

### I. Composition

One aspect of the present invention is a composition comprising a metal di-amino acid or tri-amino acid chelate complex. Such a complex comprises a metal di-amino acid or tri-amino acid chelate and, optionally, a counterion, each described in more detail below.

Metal di-amino acid or tri-amino acid chelate complexes of the present disclosure are entropically favored compared to single amino acids, and as such, are more thermodynamically stable in solution relative to such. In addition, the aqueous solubility of the metal di-amino acid or tri-amino acid chelate complexes described herein provides advantages for the commercial use and production of such complexes.

### (a) Metal Di-Amino Acid or Tri-Amino Acid Chelate

A metal di-amino acid or tri-amino acid chelate complex of the present disclosure comprises a metal di-amino acid or tri-amino acid chelate. Such a chelate comprises a di-amino acid or tri-amino acid, a metal, and optionally water, as detailed below. A metal di-amino acid or tri-amino acid chelate of the present disclosure may have a positive, negative, or neutral charge in solution.

Typically, a metal di-amino acid or tri-amino acid chelate of the present disclosure has a 1:1 or a 1:2 ratio of metal to di-amino acid or tri-amino acid ligand. In some embodiments, a 1:1 ratio metal:tri-amino acid chelate is preferred. In other embodiments, a 1:2 ratio metal:tri-amino acid chelate is preferred. In some other embodiments, a 1:1 ratio metal:di-amino acid chelate is preferred. In still other embodiments, a 1:2 ratio metal:di-amino acid chelate is preferred.

It is important to note that a di-amino acid refers to a di-peptide; this is not equivalent to a bis-amino acid ligand, which would refer to two individual amino acids (as opposed to a di-peptide). Similarly, a tri-amino acid refers to a tri-peptide; this is not equivalent to a tris-amino acid ligand, which would refer to three individual amino acids (as opposed to a tri-peptide).

i. Di-Amino Acid

A di-amino acid suitable for use in the present disclosure includes di-amino acids capable of forming at least two coordinate bonds with a metal ion. In preferred embodiments, a di-amino acid suitable for use in the present disclosure includes di-amino acids capable of forming between 2 and 6 coordinate bonds with respect to the metal.

In some embodiments, a di-amino acid suitable for use in the present disclosure forms chelate bonds at all Lewis acid locations within the di-amino acid. In some further embodiments, a di-amino acid suitable for use in the present disclosure does not form chelate bonds via carbonyl groups. In other embodiments, a di-amino acid suitable for use in the present disclosure does form chelate bonds via a carbonyl group.

Generally speaking, overall anionic, neutral or cationic complexes may be created by varying the di-amino acid. In all embodiments, D or L amino acids may be used.

In one embodiment, the di-amino acid is di-glycine, also called herein G2, 2-[(2-Aminoacetyl)amino]acetic acid, or glycylglycine. The structure of di-glycine may be represented by:

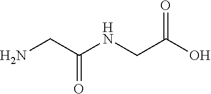

In other non-limiting embodiments, the di-amino acid may be di-aspartic acid (D2), di-glutamic acid (E₂), di-histidine (H₂), di-serine (S₂), or di-tyrosine (Y₂). In still other non-limiting embodiments, the di-amino acid may be comprised of two amino acids, each selected from the group consisting of glycine (G), aspartic acid (D), glutamic acid (E), histidine (H), serine (S), and tyrosine (Y). For instance, a di-amino acid may be GD, GE, GH, GS, GY, GG, DG, ED, DE, or other combinations.
ii. Tri-Amino Acid

A tri-amino acid suitable for use in the present disclosure includes tri-amino acids capable of forming at least two coordinate bonds with a metal ion. In preferred embodiments, a tri-amino acid suitable for use in the present disclosure includes tri-amino acids capable of forming between 2 and 6 coordinate bonds with respect to the metal.

In some embodiments, a tri-amino acid suitable for use in the present disclosure forms chelate bonds at all Lewis acid locations within the tri-amino acid. In some further embodiments, a tri-amino acid suitable for use in the present disclosure does not form chelate bonds via carbonyl groups. In other embodiments, a tri-amino acid suitable for use in the present disclosure does form chelate bonds via a carbonyl group.

Generally speaking, overall anionic, neutral or cationic complexes may be created by varying the tri-amino acid. In all embodiments, D or L amino acids may be used.

In one embodiment, the tri-amino acid is tri-glycine, also called herein G3, 2-[[2-[(2-aminoacetyl)amino]acetyl]amino]acetic acid, or glycylglycylglycine. In other non-limiting embodiments, the tri-amino acid may be tri-aspartic acid (D3), tri-glutamic acid (E3), tri-histidine (H3), tri-serine (S3), or tri-tyrosine (Y3). In still other non-limiting embodiments, the tri-amino acid may be comprised of three amino acids, each selected from the group consisting of glycine (G), aspartic acid (D), glutamic acid (E), histidine (H), serine (S), and tyrosine (Y). For instance, a tri-amino acid may be GDG, GGD, DGG, EDG, GDE, or other combinations.

iii. Metal

The metal of the metal di-amino acid or tri-amino acid chelate of the present disclosure has an oxidation state of +2 or +3. In some embodiments, the metal is an essential metal for the health of an organism. In certain embodiments, the metal may be magnesium(II), calcium(II), zinc(II), Fe(II), or a combination thereof. In one embodiment, the metal may be magnesium(II). In another embodiment, the metal may be calcium(II). In still another embodiment, the metal may be zinc(II). In some embodiments, the metal may be Fe(II) or Fe(III).

iv. Water

A metal di-amino acid or tri-amino acid chelate of the present disclosure may comprise one or more water molecules. In one embodiment, a metal di-amino acid or tri-amino acid chelate may comprise at least one water molecule. In another embodiment, a metal di-amino acid or tri-amino acid chelate may comprise at least two water molecules. In still another embodiment, a metal di-amino acid or tri-amino acid chelate may comprise at least three water molecules.

v. Specific Structures

In particular embodiments, a metal tri-amino acid chelate may have one of the following five structures:

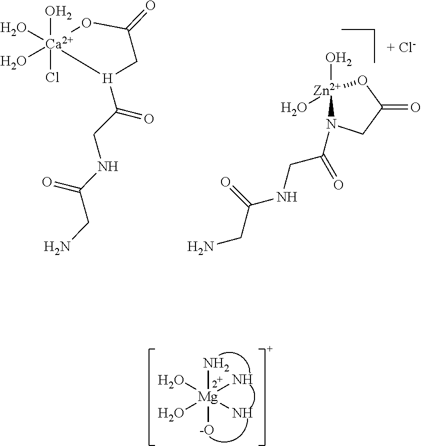

In another embodiment, a metal tri-amino acid chelate of the present disclosure is a chelate of magnesium and tri-glycine. For example, a metal tri-amino acid chelate of the present disclosure may be magnesium 2-[[2-[(2-aminoacetyl)amino]acetyl]amino]acetic acid chelate, at a 1:1 ratio. In still another embodiment, a metal tri-amino acid chelate of the present disclosure is a 1:1 or a 1:2 zinc tri-glycine chelate. In yet another embodiment, a metal tri-amino acid chelate of the present disclosure is a 1:1 or a 1:2 calcium tri-glycine chelate. In still yet another embodiment, a metal tri-amino acid chelate of the present disclosure is a 1:1, 1:2, or 1:3 iron tri-glycine chelate.

In still other embodiments, a metal di-amino acid chelate of the present disclosure is a chelate of magnesium and di-glycine. For example, a metal di-amino acid chelate of the present disclosure may be magnesium 2-[(2-aminoacetyl)amino]

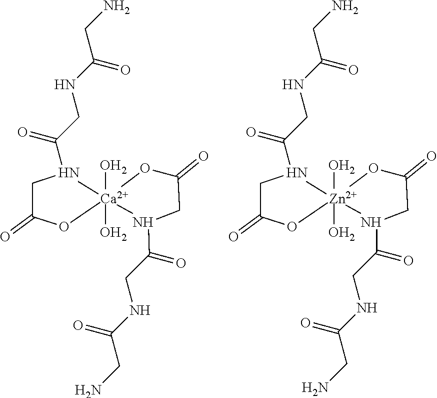

acetic acid chelate, at a 1:1 ratio. In still another embodiment, a metal di-amino acid chelate of the present disclosure is a 1:1 or a 1:2 zinc di-glycine chelate. In yet another embodiment, a metal di-amino acid chelate of the present disclosure is a 1:1 or a 1:2 calcium di-glycine chelate. In still yet another embodiment, a metal di-amino acid chelate of the present disclosure is a 1:1, 1:2, or 1:3 iron di-glycine chelate.

### (b) Counterion

A metal di-amino acid or tri-amino acid chelate complex of the present disclosure comprises a counterion. This counterion balances the charge of the metal di-amino acid or tri-amino acid chelate ion. Suitable counterions may include organic or inorganic anions or cations with an appropriate charge to balance the charge on the chelate ion. In some embodiments, the counterion is hydroxide. In other embodiments, the counterion is chloride. The counterion, along with the metal di-amino acid or tri-amino acid chelate ion, forms a neutrally charged complex when in solid form.

### (c) Acid

A composition of the present disclosure which comprises a metal di-amino acid or tri-amino acid chelate or complex may further comprise an organic acid, an inorganic acid, or a metal organic acid complex. For instance, by way of non-limiting example, in some embodiments, a composition of the present disclosure may comprise citric acid, malic acid, acetic acid or tartaric acid.

The composition may comprise about 1, 5, 10, 15, 20, 25, 30, 35, 40, 45, or 50% organic acid. In some embodiments, the composition may comprise from about 5 to about 25% organic acid. In further embodiments, the composition may comprise form about 8 to about 12% organic acid.

### (d) Specific Complexes

In particular embodiments, a metal tri-amino acid chelate complex of the present disclosure may have the following structure:

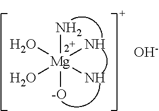

In other embodiments, a metal tri-amino acid chelate complex of the present disclosure may have the following structure:

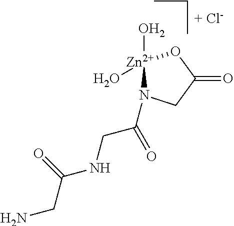

In still another embodiment, a metal tri-amino acid chelate complex of the present disclosure may have the following structure:

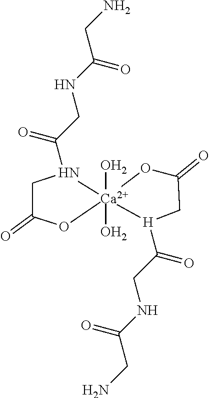

### II. Pharmaceutical Formulation

Another aspect of the present invention is a pharmaceutical formulation comprising a composition detailed in section I above. A pharmaceutical formulation may be prepared for parenteral, oral, or other suitable routes of administration, including administration via inhalation. The pharmaceutical formulation comprises a composition of section I above, as an active ingredient, and at least one pharmaceutically acceptable carrier for parenteral, oral, or topical administration. The term parenteral, as used herein, includes subcutaneous, intravenous, intramuscular, intradermal, intra-arterial, intraosseous, intraperitoneal, or intrathecal injection, or infusion techniques. The term oral, as used herein, includes sub-lingual and gavage.

The pharmaceutical formulation can be formulated into various dosage forms and administered by a number of different means that will deliver a therapeutically effective amount of the active ingredient. Such compositions can be administered in dosage unit formulations containing conventional nontoxic pharmaceutically acceptable carriers, adjuvants, and vehicles as desired. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980).

Certain embodiments of the invention relate to oral formulations and oral administration. Oral formulations generally may include an inert diluent or an edible carrier. Oral formulations may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier. Pharmaceutically compatible binding agents and/or adjuvant materials may be included as part of the composition.

The active constituent compound of a solid-type dosage form for oral administration can be mixed with at least one additive, such as sucrose, lactose, cellulose, mannitol, trehalose, raffinose, maltitol, dextran, starches, agar, alginates, chitins, chitosans, pectins, gum tragacanth, gum arabic, gelatin, collagen, casein, albumin, synthetic or semisynthetic polymer, or glyceride. These dosage forms can also contain other type(s) of additives, e.g., inactive diluting agent, lubricant such as magnesium stearate, paraben, preserving agent such as sorbic acid, ascorbic acid, alpha-tocopherol, antioxidants such as cysteine, disintegrators, binders, thickeners, buffering agents, pH adjusting agents, sweetening agents, flavoring agents or perfuming agents.

For parenteral administration, the preparation may be an aqueous or an oil-based solution. Aqueous solutions may include a sterile diluent or excipient such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as ethylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil.

The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

In certain embodiments, a pharmaceutical formulation comprising a composition of section I is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one embodiment, a pharmaceutical formulation may comprise a liposome delivery vehicle. Liposomes, depending upon the embodiment, are suitable for delivery of a composition of section I in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the composition may be selectively delivered to a cell by encapsulation in a liposome that fuses with the targeted cell's membrane.

Liposomes may be comprised of a variety of different types of phospholipids having varying hydrocarbon chain lengths. Phospholipids generally comprise two fatty acids linked through glycerol phosphate to one of a variety of polar groups. Suitable phospholipids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethylammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindocarbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which sphingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying a composition of section I above may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar liposomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size do not significantly scatter visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. A composition of section I may be encapsulated in a microemulsion by any method generally known in the art.

In yet another embodiment, a composition of section I may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

For administration by inhalation, the compounds may be delivered in the form of an aerosol spray from a pressured container or dispenser which contains a suitable propellant, e.g., a gas such as carbon dioxide, or a nebulizer.

Controlled-release (or sustained-release) preparations may be formulated to extend the activity of the chelates of section I and reduce dosage frequency. Controlled-release preparations can also be used to effect the time of onset of action or other characteristics, such as blood levels of Othe chelate, and consequently affect the occurrence of side effects. Controlled-release preparations may be designed to initially release an amount of a chelate that produces the desired therapeutic effect, and gradually and continually release other amounts of the chelate to maintain the level of therapeutic effect over an extended period of time. In order to maintain a near-constant level of a chelate in the body, the chelate can be released from the dosage form at a rate that will replace the amount of chelate being metabolized or excreted from the body. The controlled-release of a chelate may be stimulated by various inducers, e.g., change in pH, change in temperature, enzymes, water, or other physiological conditions or molecules.

### III. Method of Use

A further aspect of the present disclosure encompasses methods of using a metal di-amino acid or tri-amino acid chelate or complex as described in section I or II above. Such methods encompass administering a pharmaceutically effective dose of a composition comprising a metal di-amino acid or tri-amino acid chelate or complex to a subject.

Suitable subjects may include a rodent, a human, a livestock animal, a companion animal, or a zoological animal. In one embodiment, a subject may be a rodent, e.g., a mouse, a rat, a guinea pig, etc. In another embodiment, a subject may be a livestock animal. Non-limiting examples of suitable livestock animals may include pigs, cows, horses, goats, sheep, llamas and alpacas. In still another embodiment, a subject may be a companion animal. Non-limiting examples of companion animals may include pets such as dogs, cats, rabbits, and birds. In yet another embodiment, a subject may be a zoological animal. As used herein, a “zoological animal” refers to an animal that may be found in a zoo. Such animals may include non-human primates, large cats, wolves, and bears. In certain embodiments, a subject may be human. In particular embodiments, a subject may be deficient in a metal. For instance, a subject deficient in magnesium may be administered a magnesium chelate of the present disclosure. Similarly, a subject deficient in zinc may be administered a zinc chelate of the present disclosure, or a subject deficient in calcium may be administered a calcium chelate of the present disclosure.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by one of appropriate skill in the art.

Various states, diseases, disorders, and conditions, described herein, as well as others, can benefit from compositions and methods described herein. Generally, treating a state, disease, disorder, or condition includes preventing or delaying the appearance of clinical symptoms in a subject that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a composition described herein can occur as a single event or over a time course of treatment. For example, a delivery system composition can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

The amount of organic acid in a composition of the present disclosure may be modulated to impact the bioavailability of the metal from the metal tri-amino acid chelate.

### IV. Method of Production

Another aspect of the present disclosure encompasses methods of producing a metal di-amino acid or tri-amino acid chelate complex. Typically, such methods comprise (i) creating an aqueous solution of a di-amino acid or tri-amino acid, a metal compound, and optionally an organic acid, (ii) stirring the solution of step (i) while optionally heating the solution up to about 90° C. for a minimum of 10 min, and (iii) precipitating the metal di-amino acid or tri-amino acid chelate complex from the solution.

Suitable metal compounds may include water soluble metal oxides and metal salts.

### (a) Creating a Solution of a Di-Amino Acid or Tri-Amino Acid, a Metal Compound, and Optionally an Organic Acid

A method of producing a metal di-amino acid or tri-amino acid chelate complex of the present disclosure comprises creating a solution of a di-amino acid or tri-amino acid, a metal compound, and optionally an organic acid. Typically, the solution is an aqueous solution. In one embodiment, an aqueous solution of a di-amino acid or tri-amino acid is prepared separately from an aqueous solution of a metal compound and optionally an organic acid, and then the two solutions are combined to create an aqueous solution of a tri-amino acid, a metal oxide, and optionally an organic acid or di-amino acid, a metal oxide, and optionally an organic acid. In another embodiment, a metal compound is directed dissolved in an aqueous solution of a di-amino acid or tri-amino acid. The optional organic acid may be added at any point.

In certain embodiments, the separate solutions are heated before they are combined. For instance, in some embodiments, each separate solution is heated to about 65, 70, 75, 80, 85, 90, 95, 100, or 105° C. before the solutions are combined. In certain embodiments, each separate solution is heated to about 85-95° C. before the solutions are combined.

A skilled artisan would appreciate that the molar equivalents of the di-amino acid or tri-amino acid and metal compound can vary. In some embodiments, the aqueous solution has a 1:1 ratio of moles of tri-amino acid to moles of metal compound. In other embodiments, the aqueous solution has from about a 3:1 to 1:3 ratio of moles of tri-amino acid to moles of metal compound. For instance, the aqueous solution may have from about a 3:1, 2.5:1, 2:1, 1.5:1, 1:1, 1:1.5, 1:2, 1:2.5, or 1:3 ratio of moles of tri-amino acid to moles of metal compound. In certain embodiments, the aqueous solution has a 1:1 ratio of moles of di-amino acid to moles of metal compound. In other embodiments, the aqueous solution has from about a 3:1 to 1:3 ratio of moles of di-amino acid to moles of metal compound. For instance, the aqueous solution may have from about a 3:1, 2.5:1, 2:1, 1.5:1, 1:1, 1:1.5, 1:2, 1:2.5, or 1:3 ratio of moles of di-amino acid to moles of metal compound.

In some embodiments, the organic acid may be present in less than or equal to 0.5, 0.4, 0.3, 0.2, or 0.1 molar equivalents compared to the metal compound. For instance, in some embodiments, the organic acid is present in less than or equal to about 0.3, 0.25, 0.2, 0.15, or 0.1 molar equivalents compared to the metal compound. In other embodiments, the organic acid is present in less than or equal to 0.12, 0.11, 0.1, 0.09, 0.08, 0.07, 0.06, 0.05, 0.04, 0.03, 0.02, or 0.01 molar equivalents compared to the metal compound.

### (b) Stirring and Optionally Heating the Solution

A method of producing a metal di-amino acid or tri-amino acid chelate complex of the present disclosure further comprises stirring and optionally heating the aqueous solution. Methods of stirring and optionally heating solutions are known in the art. In some embodiments, the solution is heated to about 25, 30, 35, 40, 45, 50, 55, 60, 65, 70, 75, 80, 85, 90, 95, 100, or 105° C. with stirring. In certain embodiments, the solution is heated to between about 75° C. and about 105° C. with stirring. In other embodiments, the solution is heated to between about 85° C. and about 95° C. with stirring. In some embodiments, the solution is heated to between about 50° C. and about 70° C. with stirring. In particular embodiments, the solution is heated to between about 55° C. and 65° C. with stirring.

Generally speaking, the aqueous solution is heated and stirred for at least about 10 min. For instance, in some embodiments, the aqueous solution is heated and stirred for about 10, 15, 20, 25, 30, 35, 40, 45, 50, 55, 60, 65, 70, 75, 80, 85, 90, 95, 100, 105, 110, 115, 120, 125, 130, 135, 140, 145, or 150 min.

(c) precipitating the Metal Di-Amino Acid or Tri-Amino Acid Chelate Complex

A method of producing a di-amino acid or metal tri-amino acid chelate complex of the present disclosure also comprises precipitating the metal di-amino acid or tri-amino acid chelate complex. Methods of precipitating chelate complexes from aqueous solutions are known in the art. In some embodiments, an alcohol may be used to precipitate the metal di-amino acid or tri-amino acid chelate complex. By way of non-limiting examples, ethanol or isopropanol may be used.

In certain embodiments, the sample may be centrifuged to pellet the precipitated chelate complex. Such a precipitate may also be washed and/or dried using methods known in the art. For instance, a precipitate may be filtered from a solution, spray dried, freeze dried, or the solution may be evaporated from the precipitate via heat or vacuum. For details of specific, non-limiting methods, please see the Examples below.

### V. Product by Process

A further aspect of the present disclosure encompasses a product produced using a method described in Section IV above. Such a product has the characteristics described in Section I above.

### EXAMPLES

### Example 1

### Synthesis of a Tri-Glycine Magnesium Chelate

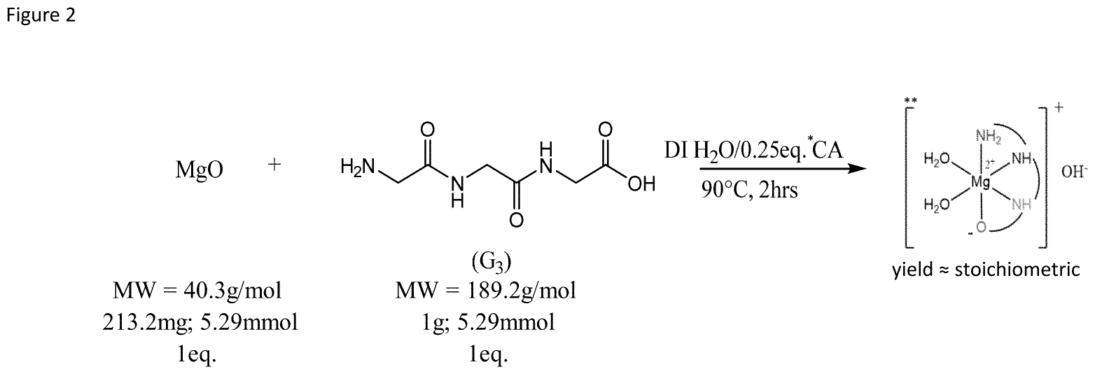

A 1.0025 g sample of triglycine (G3—5.29 mmol; 1 eq.) was dissolved in 10 mLs of 18 MΩ H₂O in a 50 mL round-bottom flask, with constant stirring at 90° C. A separate solution of 215.5 mgs magnesium oxide (MgO—5.29 mmol; 1 eq.) was taken up in 10 mLs of DI H₂O, with an addition of 253.6 mgs of citric acid (CA—0.25 eq), constantly stirred and heated to 90° C. The MgO/CA solution was subsequently added to the triglycine solution. Immediately upon addition, the combined solution turned a milky white color, whereupon this became a clear solution after ˜20 minutes with stirring at 90° C. The reaction was run for 2 hrs, and then cooled to room temperature. The pH of the solution was noted as 10.2. The solution was concentrated down to approximately 3 mLs and a white solid was precipitated with anhydrous ethanol. The solid was isolated by centrifugation at 4000 rpm at room temperature for 10 minutes, and the ethanol decanted off. The solid was triturated with diethyl ether, recentrifuged as before, the ether decanted off, and the solid dried in vacuo overnight. The dried white material was collected, and the yield obtained. An illustration of the reaction may be found in FIG. 2.

Magnesium oxide (99.99% metals basis) was purchase through Fisher Scientific. Triglycine (Gly-Gly-Gly, BioUltra™, ≥99.0%) and citric acid (ACS Reagent, ≥99.5%) were purchased through Sigma-Aldrich. 18 MΩ Ultrapure water was obtained in-house. D₂O and DMSO-d6 NMR solvents were purchase from Sigma-Aldrich. Potassium bromide (KBr) for FT-IR analysis was purchased from Sigma-Aldrich. Magnesium uptake colorimetric assay kits were purchased from BioVision (Catalog #385-100). Stock solutions for magnesium uptake assays were made in-house, with MgCl₂ (BioReagent, ≥97.0%) purchased from Sigma-Aldrich and magnesium bisglycinate (MgBG) provided by Balchem Corp (New Hampton, NY) and confirmed for purity in-house by ¹H NMR. HEK-293 (ATCC® CRL-1573TM) cells, Caco-2 (ATCC® HTB-37TM) cells, and Dulbecco's Modified Eagle Medium (ATCC® 30-2002) were purchased from ATCC. Penicillin streptomycin (10,000 U/mL), fetal bovine serum (FBS), and trypsin/EDTA (0.25%) were purchased from Gibco™. Clear 96-well Armadillo PCR assay plates (Catalog #AB2396) were purchased from ThermoFisher.

### Example 2

### Impact of Organic Acid on Synthesis

As detailed above, an organic acid may be used as a reactant in the method of producing a metal chelate of the present disclosure. The amount of organic acid included as a reactant is important to establish full conversion to product. By modifying the organic acid concentration, it is possible to modulate the amount of metal-organic acid salt contained in the final product. For instance, see the table below, which illustrates that increasing organic acid molar equivalents result in increased citric acid production in the context of making a magnesium chelate.

Quantifying Mg Citrate with Increasing Citric Acid Concentration

| NMR | Amount of | pH of | % Mg Citrate | % citric acid in |
|---|---|---|---|---|
| sample | Citric Acid (eq) | solution | production | final solution |
| 1 | 0.01 (2 hrs) | 9.2 | 0% | 0% |
| 2 | 0.1 (2 hrs) | 10.03 | 0% | 1-3% |
| 3 | 0.25 (2 hrs) | 10.2 | 0% | 4-6% |
| 4 | Citrate Buffer | 10.4 | 0% | 9-11% |
| | (2hrs) − 0.4 | | | |

Increasing the amount of citric acid present also prompts the formation of magnesium citrate (solid recovered from citrate buffer reaction analyzed via FT-IR). The increase in pH seen with increasing amounts of citric acid is expected given the production of a citrate chelate. Citric acid is observed at concentrations as low as 0.1 molar equivalents (relative to MgO). As a result, if no NMR quantifiable level of citric acid is desired, less than 0.1 molar equivalents (relative to MgO) should be used.

If a mixed species of the tri-chelate with metal-organic acid complex is desired, longer reaction times at high M eq of the organic acid can be used. For example, utilizing 0.25 M eq of citric acid, relative to MgO, at 90 C for 24 hours will produce a ˜90% magnesium tri-amino acid chelate with ˜10% magnesium-citric acid.

### Example 3

### Characterization of Tri-Glycine Magnesium Chelate

Electrospray ionization mass spectroscopy was carried out on a Shimadzu LC-MS 8040 LC-MS/MS—samples were analyzed utilizing a solvent system of H₂O/MeOH/0.1% TFA at a flow rate of 0.2 mLs/min over a 1.5 min time frame and evaluated from 0-600 m/z. 1D- and 2D-NMR were conducted on a Bruker Avance 400 MHz instrument. FT-IR was carried out on a Nicolet Infrared Spectrophotometer (64 scans with background subtracted) as KBr pellets. TGA was carried out on (insert model here) from 20° C.-800° C. with the subsequent DSC being carried out on (insert model here) from 30° C.-400° C. Elemental analysis was conducted by Intertek Pharmaceutical Services (Whitehouse, N.J., US).

### Electrospray Ionization Mass Spectroscopy

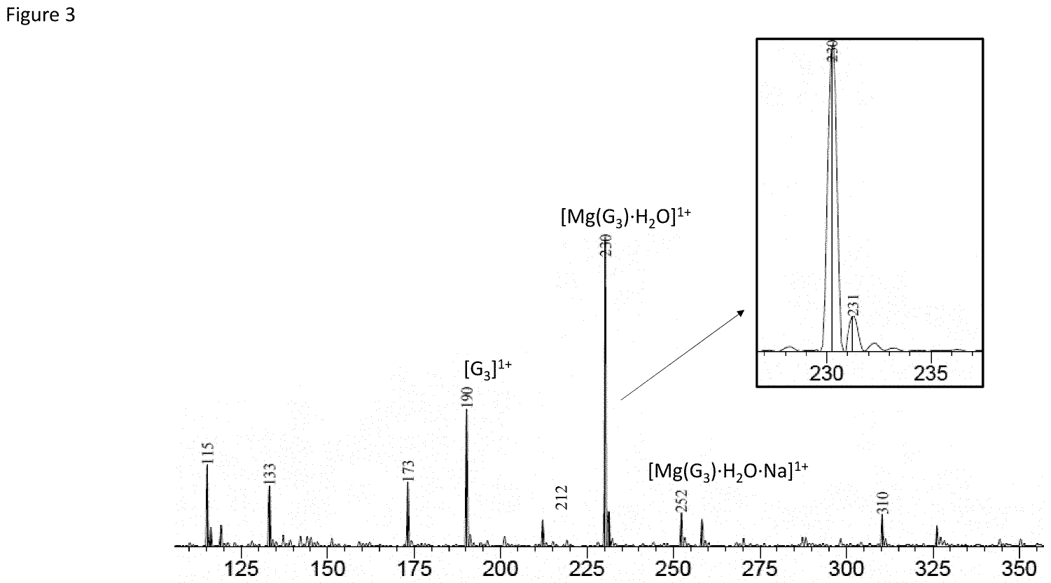

To determine presence of desired product from the synthesis of Mg(G3), a preliminary mass spectrum of the dried Mg(G3) resuspended in 18MC) H₂O indicated the presence of the free [HG3]¹⁺ ligand and the subsequent sodium adduct [G3.Na]+predicted at 190 m/z and 212 m/z respectively, as well as presence of the monoaqua triglycine magnesium chelate [Mg(G3).H₂O]+ and the subsequent sodium adduct [Mg(G3).H₂O.Na]+ at 230 m/z and 252 m/z respectively as indicated by FIG. 3. While preliminary mass spec does indicate presence of desired product, it does not give any indication of how the G3 is coordinated to magnesium and subsequent ¹H NMR analyses were conducted.

Structural Characterization of Mg(G3) via ¹H NMR and 2D ¹H/¹³C HSQC/HMBC NMR

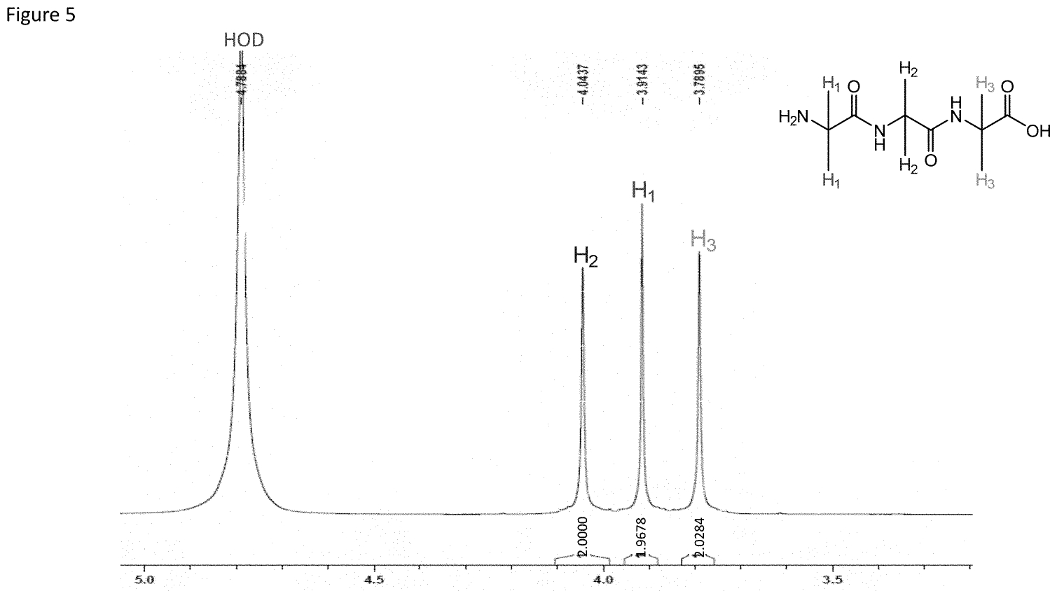

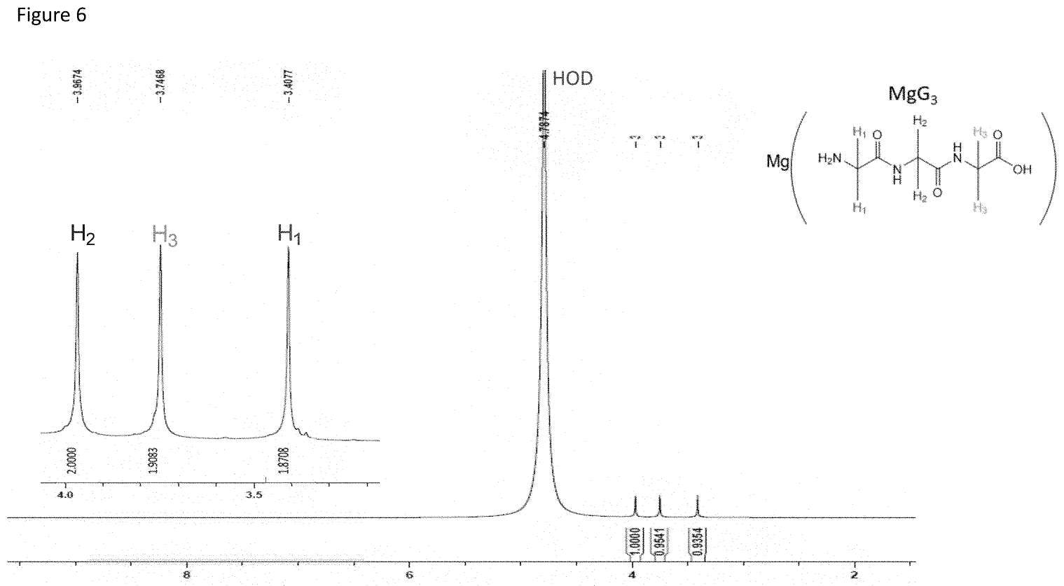

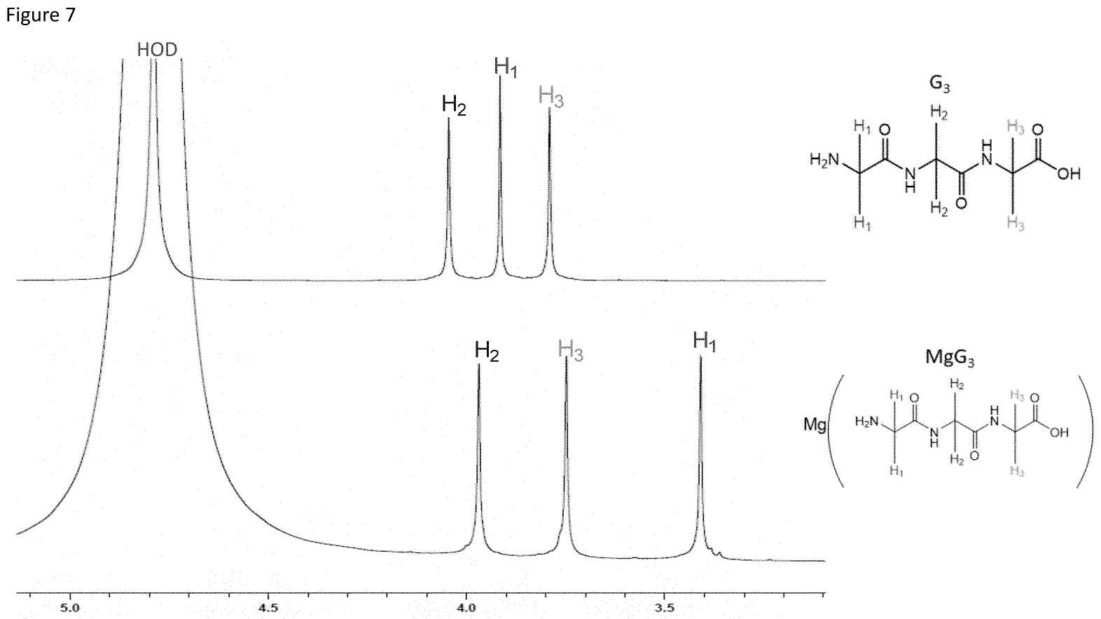

¹H NMR was conducted on both the free HG3 ligand and the presumed pure and dried Mg(G3) in 700 μl of 18MC) H₂O/D₂O (1:6 (v:v)). The free HG3 exhibited three singlets at 4.044, 3.914, and 3.786 ppm, whereas the complex Mg(G3) showed an observable upfield shift for each of the proton peaks at 3.967, 3.747, 3.4077 respectively (FIGS. 5 and 6) indicating an upfield shift of 0.5 ppm for H₁, 0.1 ppm for H₂, and 0.04 ppm of H₃ (FIG. 7), which signifies that all available Lewis bases are participating in magnesium chelation.

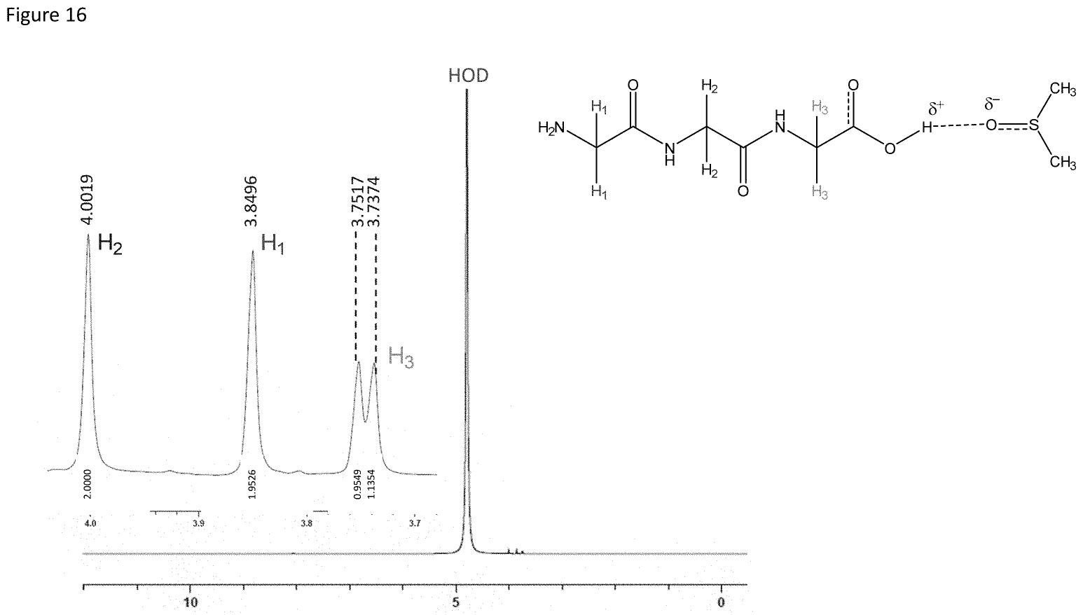

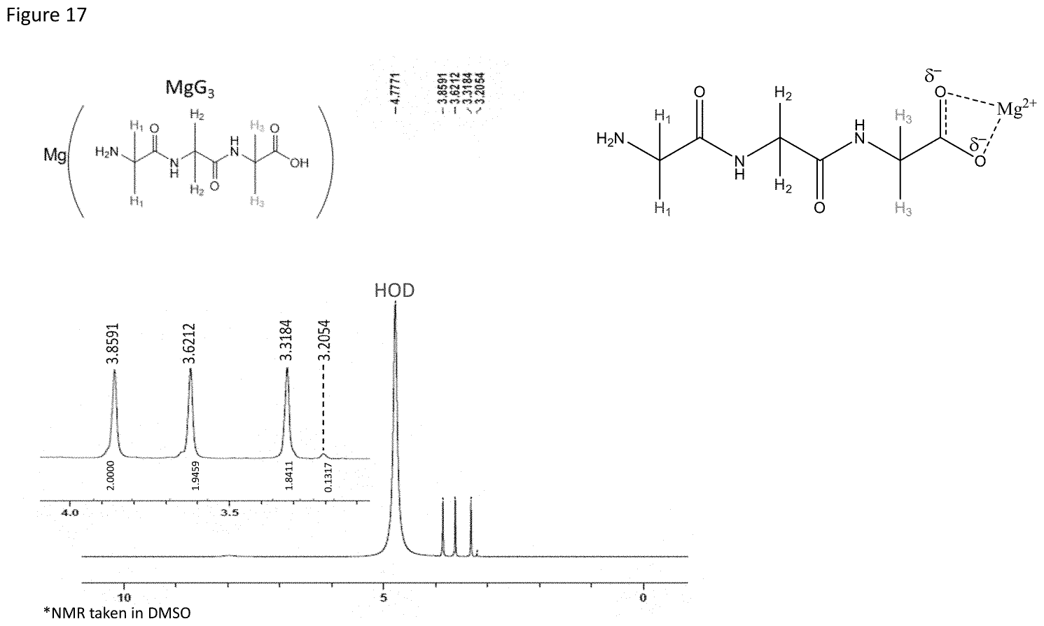

With the understanding that the triglycine ligand will most likely coordinate through the carboxylic acid moiety, subsequent ¹H NMR in a solution of 700 μl of DMSO-d₆/H₂O (1:6 (v:v)) was conducted to determine the coordinating participation of this moiety. It was hypothesized that the free proton of the carboxylic acid, when in the presence of the polar aprotic DMSO, would participate in hydrogen bonding, thus resulting in an observable splitting of the proton signal; when coordinated, the HG3 ligand would lose this carboxylic proton, thus eliminating the presence of an observable proton split. Subsequent ¹H NMR supported this hypothesis, thus implicating the carboxylic acid moiety as a participant in coordination to the magnesium metal (FIGS. 16 and 17).

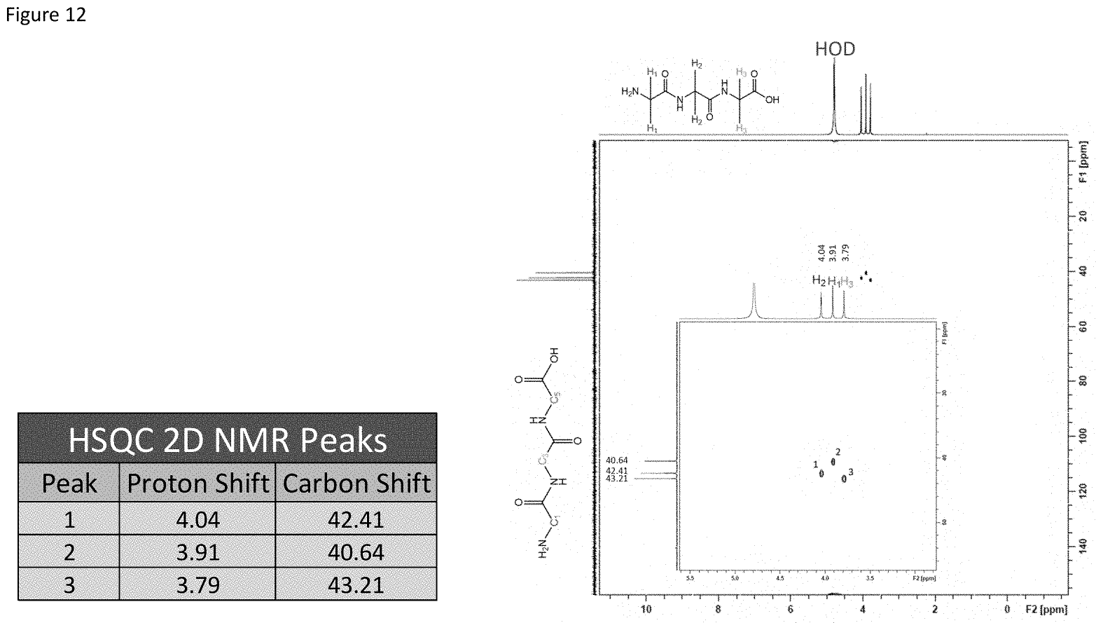

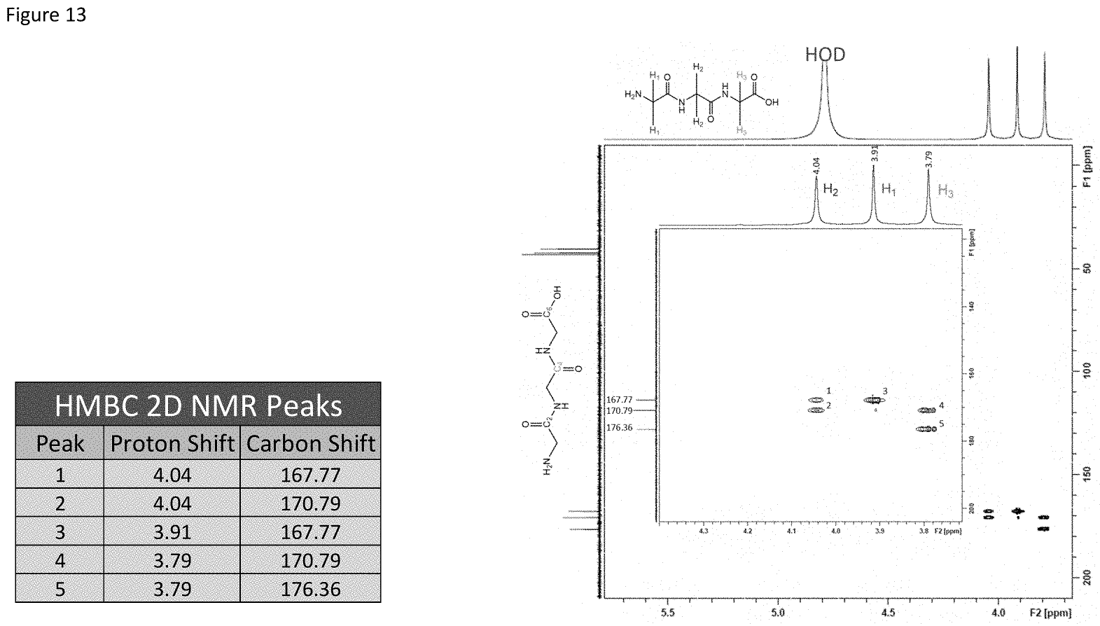

While 1-Dimensional ¹H NMR is indicative of HG3 chelation to the magnesium metal, it does not indicate how each proton environment adjacent to each coordinating Lewis base is impacted, or the overall coordination geometry of the Mg(G3) complex. To glean further information into the coordinating moieties of the HG3 ligand, 2D heteronuclear single quantum coherence (HSQC) and heteronuclear multiple bond correlation (HMBC) were utilized. Initial HSQC and HMBC were taken of the free HG3 ligand (FIGS. 12 and 13).

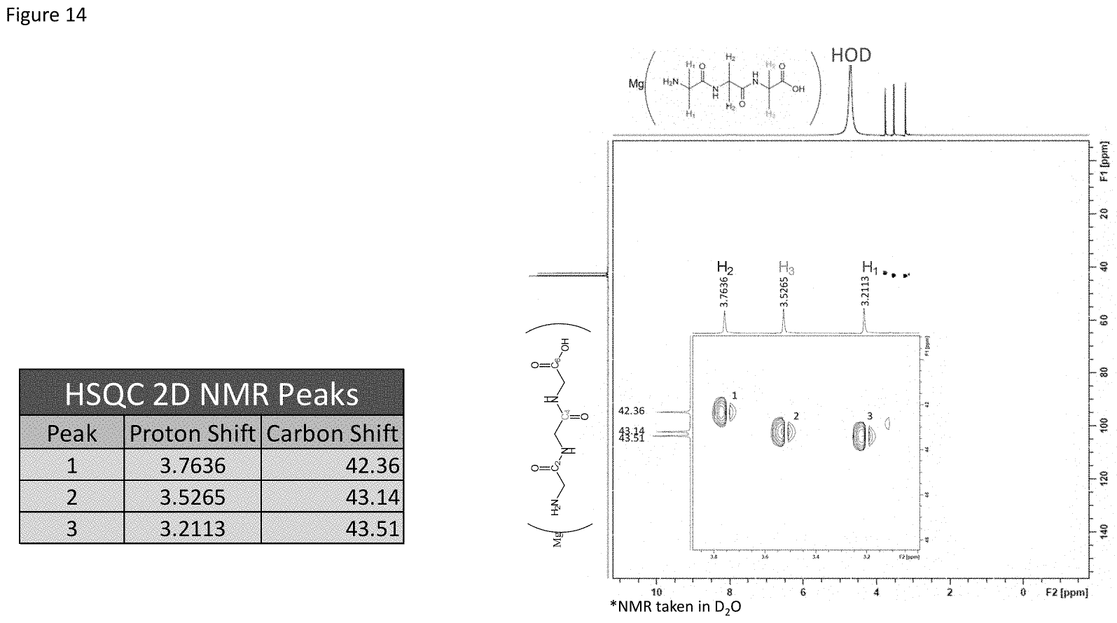

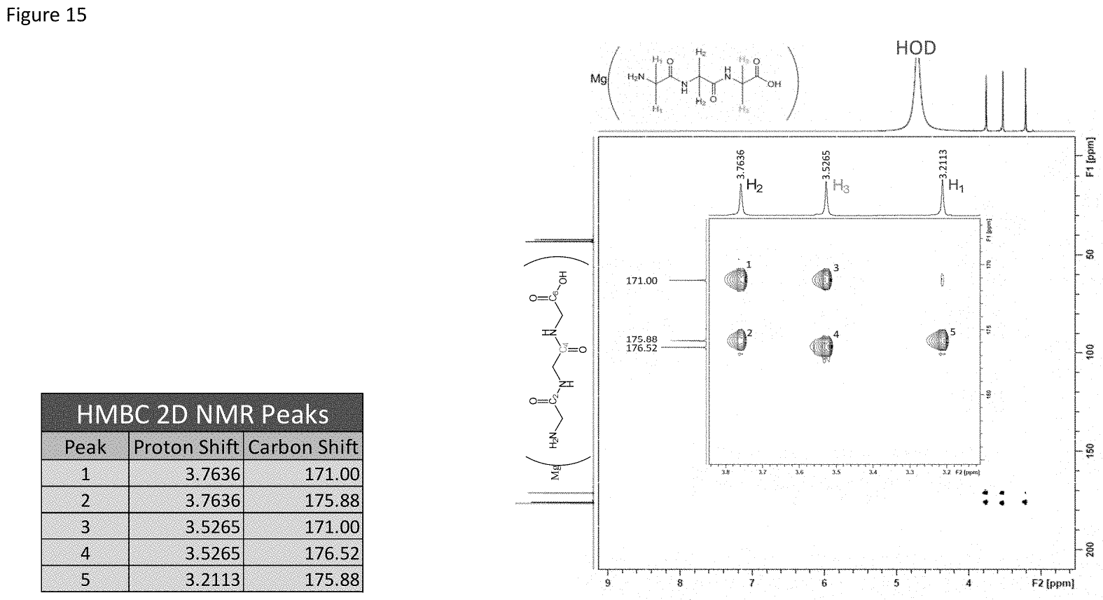

Subsequently, 2D HSQC and HMBC were conducted on the dried Mg(G3). The 2D spectra were analyzed for any change in the patterns of the nuclei points observed. The most significant change in these nuclei was observed for the H3 proton (FIGS. 14 and 15), which suggests that the terminal amine is involved in magnesium chelation. The combination of both the 1D and 2D ¹H NMR suggests that all available Lewis bases are coordinated to the magnesium metal, implicating HG3 as a possible tetradentate chelate.

Determination of the Chemical Composition of Mg(G3) via TGA/DSC and Elemental Analysis

It is well known that magnesium exhibits an extensive hydration sphere and typically assumes a six-coordinate octahedral geometry. It was hypothesized, given excess water reaction conditions, and the assumed tetradentate coordination mode of the HG3 ligand, that the remaining two coordination sites of the magnesium would be occupied by water, and charge balance is achieved via a hydroxy anion. To validate this hypothesis, solid state characterization via thermogravimetric analysis (TGA), differential scanning calorimetry (DSC), and elemental analysis were employed.

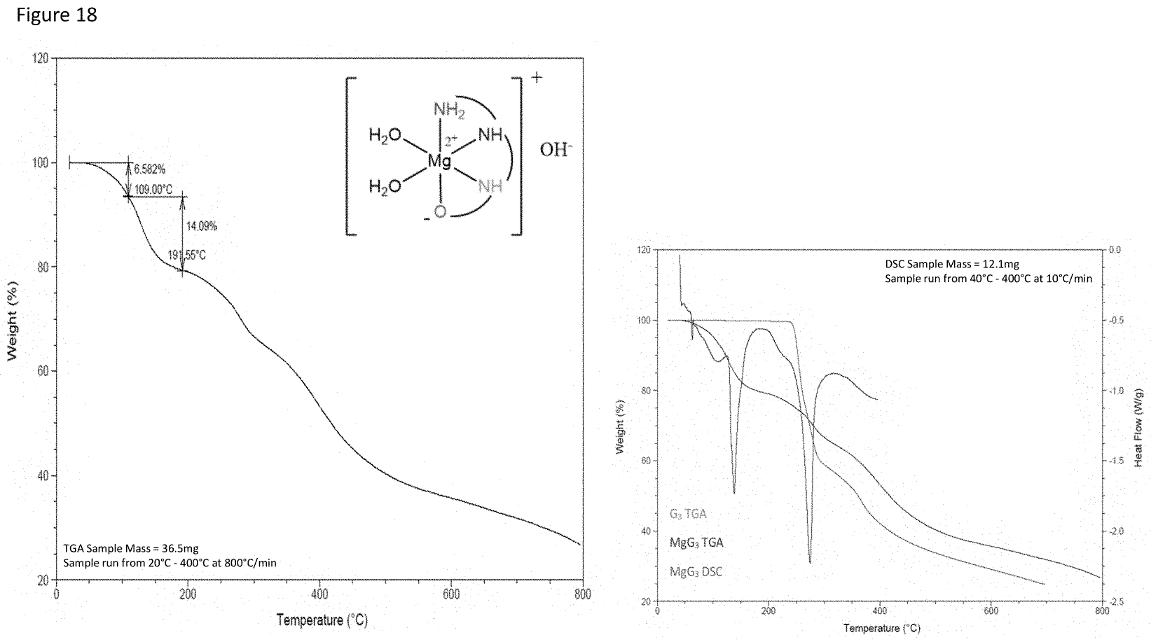

The TGA of Mg(G3) was strikingly different than that of the free HG3 ligand, which exhibited only one continuous weight percent change beginning at approximately 240° C. Mg(G3) exhibited three weight percent changes: 6.6% (calculated to 6.4%) at 109° C., 14.1% (calculated to 14.5%) at 191.5° C., and one continuous change at approximately 240° C. The first weight percent change corresponds to loss of a hydroxy anion (as was confirmed via electrochemistry), the second weight percent change corresponds to the loss of two coordinating waters from a parent complex of [Mg(G3)(H₂O)₂]OH, and the last corresponds to the degradation of the free G3 ligand. These three independent events were further supported by the presence of three separate observable endotherms on the DSC at 109° C., 191.5° C., and 240° C. (FIG. 18).

The chemical composition of Mg(G3) was further verified via elemental analysis through Intertek Pharmaceuticals (NJ, USA); replicate analyses were conducted to confirm percent composition. Analysis confirmed a complex of [Mg(G3)(H₂O)₂] (Theoretical: C=29.00%, H=5.68%, N=16.90%; Experimental: C=29.22%, H=6.22%, N=16.03%). The elemental analysis further supports the hypothesis that there is a hydroxy anion because this anion does not show up in the elemental analysis as an in situ artifact, but does show up in the TGA/DSC, suggesting that the supplemental mass is not due to subsidiary coordinating water ligand, but a non-complexed hydroxy anion required for charge balance.

Culturing of Caco-2 and HEK293 Cells

Caco-2 cells were cultured from Liquid N₂ frozen stocks and rapidly thawed to RT using a water bath at 37° C.; cryopreservation media was removed with a micropipette after cells were pelleted via centrifugation for 8-10 minutes at 125 g. Cells were resuspended in 1 mL of room temperature DMEM and cultured in 14 mLs DMEM (total volume of 15 mLs) with a seeding density of 3.6×104 cells/cm² in a T-75 cm² culture flask and left to grow in an incubator at 37° C. and 5% CO₂. Cells were sub-cultured at 90% confluency, and sub-culturing occurred a minimum of five times before use in uptake assays. When ready-to-use cultures were at 90% confluency, old DMEM media was pipetted off and 3-4 mLs of trypsin was added—the trypsinized culture flask was placed back in the incubator for 5-10 minutes to detach cells. Once detached, the culture flask was rinsed with 6-8 mLs of fresh DMEM to neutralize the trypsin -cells for uptake assays were counted utilizing a DeNovix CellDrop™. Once counted, cells were pelleted down via centrifugation at 125 g for 8-10 minutes, and the neutralized trypsin-media supernatant was pipetted off. Cells were resuspended in 5 mLs magnesium assay buffer for cellular uptake assay.

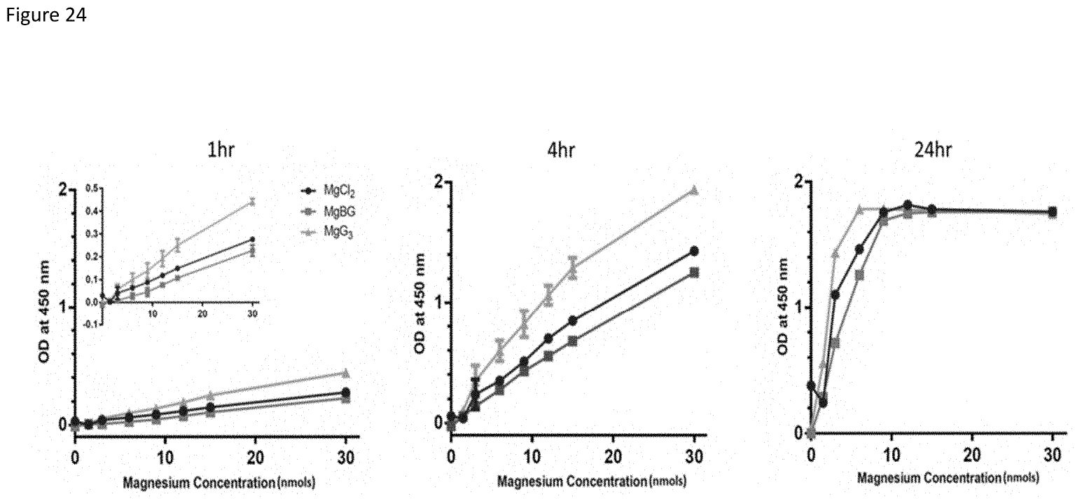

Cellular uptake of Mg(G3) was evaluated in Caco-2 cells relative to MgCl₂ and Balchem's magnesium bisglycinate (MgBG) utilizing a BioVision colorimetric magnesium uptake assay kit. Cellular uptake data was collected at incubation times of 1 hr (required kit incubation time), 4 hrs (the amount of time required for uptake in the GI), and 24 hrs (the amount of time required for a complex to clear the GI). It was hypothesized that the exceptional solubility of Mg(G3) (determined to be 150 g/100 mL), that stems from the coordination of the triamino acid G3, would result in increased bioavailability and a subsequent increase in cellular uptake. Collected data indicates that Mg(G3) exceeds the cellular uptake seen with the standard magnesium salt MgCl₂ at a significantly lesser percent composition of magnesium (9%, 14%, and 25% respectively). This greater level of uptake was observed at both 1 and 4 hrs. At 24 hrs, data indicated that cell saturation had occurred (FIG. 24)

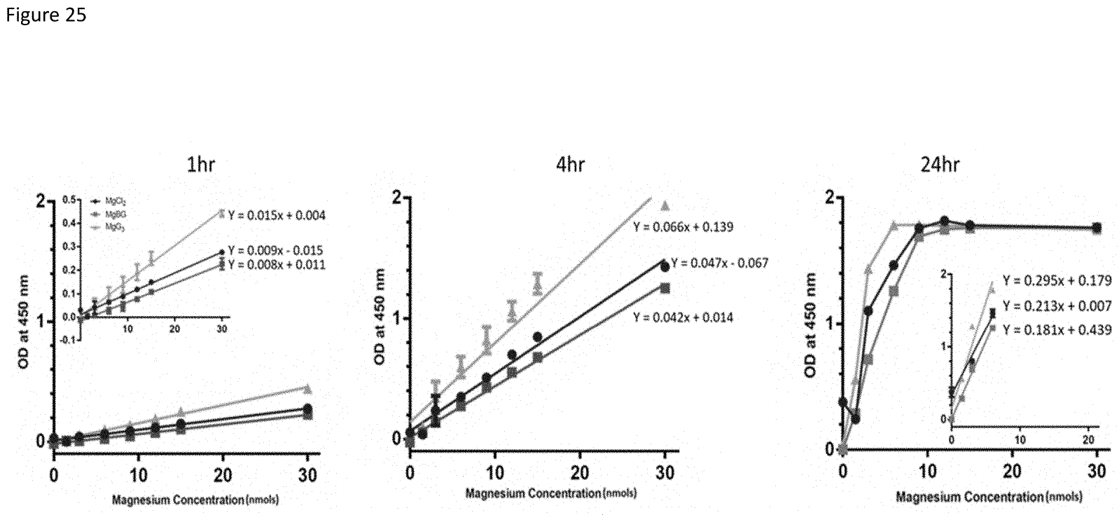

Kinetic evaluation of cellular uptake was also conducted to determine how uptake of Mg(G3) compared to that of MgBG and MgCl₂. Kinetics were evaluated utilizing the slope of the line of uptake of the complex in the Caco-2 cells. Kinetics of uptake were evaluated predominantly at 1 hr and 4 hr—kinetics evaluation at 24 hrs was not pertinent due to the observable saturation of the cells indicated by the plateau of the uptake curve at magnesium concentrations greater than 10 nmols. At 1 hr, Mg(G3) exhibited uptake approximately 1.6× faster than MgCl₂, and approximately 1.9× faster than that of MgBG. At 4 hrs, Mg(G3) showed uptake that was approximately 1.4× faster than that of MgCl₂, and approximately 1.5× faster than that of MgBG (FIG. 25).

Coordination of magnesium to triglycine increased magnesium solubility significantly; the solubility of Mg(G3) was found to be approximately 150 g/100 mLs H₂O. This increase in solubility is attributed to the inherently high water-solubility of the triglycine, as well as the ionic nature of the overall complex. The solubility of Mg(G3) is approximately 3×'s more soluble than the next closest magnesium supplement (magnesium chloride) and as such shows great promise for cellular uptake, as this increase in solubility will aid in bioavailability. For instance, Mg(G3) shows greater cellular uptake than both MgCl₂ and MgBG at a lesser overall magnesium percent composition.

Elemental Analysis of Mg(G3) suggests 1:1 stoichiometry between G3 and magnesium (see table below). DSC and TGA suggest an [Mg(G3)(H₂O)₂]⁺OH− diaquo species. Values for samples are within accepted error range. The absence of hydroxide mass supports the claim that the hydroxide is in the lattice.

Elemental Analysis Values

| Mg(G3) | Carbon % | Nitrogen % | Hydrogen % |
|---|---|---|---|
| Calculated Values | 29.00% | 16.9% | 5.68% |
| Sample 1 | 29.25% | 16.03% | 6.31% |
| Sample 2 | 29.19% | 16.02% | 6.12% |
| Average Mass % | 29.22% | 16.03% | 6.22% |

Upfield shifts of all protons, specifically the Hi proton suggest multiple chelating locations of the G3 ligand outside of just the carboxylic acid. Downfield shifts of coupled carbons at proton locations further suggests multiple chelating locations of G3 ligand. These shifts indicate chelation at all Lewis acid locations, suggesting a tetradentate G3. The data also suggests that carbonyls are not involved in chelation.

Proton NMR of G3 and Mg(G3) run in DMSO show splitting of H₃ proton due to the hydrogen bonding participation of the carboxylic acid proton and the DMSO. This comes about from the H₃ protons feeling two separate environments at the hydrogen-bonded carboxylic acid moiety. Disappearance of H₃ splitting in chelate suggests the removal of the carboxylic acid proton, thus chelation occurs at this point. The carbonyls are not involved in chelation.

Cation exchange chromatography suggests that the complex is positively charged. Elemental analysis, TGA, and DSC indicate that there are two waters coordinated to the metal and a hydroxide in the lattice, giving rise to a neutral complex in the solid state and a positively charged complex in solution.

### Example 4

### Synthesis of Calcium Triaqua Triglycine Chloride (1:1)

A 1.0033 g sample of triglycine (G3—5.29 mmol; 1 eq.) was dissolved in approximately 10 mLs DI H₂O in a 50 mL round-bottom flask, with constant heating and stirring at 60° C. A separate solution of 587.9 mgs calcium chloride (CaCl₂—5.29 mmol; 1 eq.) was taken up in approximately 10 mLs DI H₂O, constantly stirred and heated to 60° C. The CaCl₂ solution was added to the triglycine solution—upon addition, the combined solution was colorless. The reaction was left to run for 1 hr at 60° C. The reaction was cooled to room temperature and filtered through a Buchner funnel (no solid was observed on the filter paper). The pH of the solution was found to be 6.02. The solution was concentrated down to approximately 3mLs and the solid was crashed out with ethanol. Centrifugation was employed to pellet the solid, and the ethanol was decanted off. The solid was washed with diethyl ether to remove the ethanol. The sample was then recentrifuged, the ether decanted off, and the solid dried in vacuo overnight. The dried material was collected and massed. Yield was found to be nearly stoichiometric.

### Example 5

### Synthesis of Calcium Monoaqua Bistriglycine Chloride (1:2) Complex

A 1.0014 g sample of triglycine (G3—5.29 mmol; 1 eq.) was dissolved in approximately 10 mLs DI H₂O in a 50 mL round-bottom flask, with constant heating and stirring at 60° C. A separate solution of 294.6 mgs calcium chloride (CaCl₂—5.29 mmol; 1 eq.) was taken up in approximately 10 mLs DI H₂O, constantly stirred and heated to 60° C. The CaCl₂solution was added to the triglycine solution—upon addition, the combined solution was colorless. The reaction was left to run for 1 hr at 60° C. The reaction was cooled to room temperature and filtered through a Buchner funnel (no solid was observed on the filter paper). The pH of the solution was found to be 6.77. The solution was concentrated down to approximately 3 mLs and the solid was precipitated with ethanol. Centrifugation was employed to pellet the solid, and the ethanol was decanted off. The solid was washed with diethyl ether to remove the ethanol. The sample was then recentrifuged, the ether decanted off, and the solid dried in vacuo overnight. The dried material was collected and massed. Yield was found to be nearly stoichiometric.

### Example 6

### Characterization of CaG3 Chelates

NMR analyses suggest reaction completion in as little as 1 hour. NMR analysis also suggests that the G3 ligand coordinates through both the terminal acid, which is subsequently deprotonated, and the adjacent nitrogen, to form a five-member ring structure. Chloride is believed to be the anion present, similar to ZnG3 reactions. TGA/DSC and elemental analysis indicate a calcium triaqua triglycine chloride (1:1) and calcium monoaqua bistriglycine chloride (1:2) complex.

### Example 7

### Synthesis of 1:1 ZnG3 Chelate

A 1.0007 g sample of triglycine (G3—5.29 mmol; 1 eq.) was dissolved in approximately 10 mLs DI H₂O in a 50 mL round-bottom flask, with constant heating and stirring at 60° C. A separate solution of 725.8 mgs zinc chloride (ZnCl₂—5.29 mmol; 1 eq.) was taken up in approximately 10 mLs DI H₂O, constantly stirred and heated to 60° C. The ZnCl₂ solution was added to the triglycine solution—upon addition, the combined solution was colorless. The reaction was left to run for 1 hr at 60° C. The reaction was cooled to room temperature and filtered through a Buchner funnel (no solid was observed on the filter paper). The pH of the solution was found to be 4.92. The solution was concentrated down to approximately 3 mLs and the solid was precipitated with isopropanol. Centrifugation was employed to pellet the solid, and the isopropanol was decanted off. The solid was washed with diethyl ether to remove the isopropanol. The sample was then recentrifuged, the ether decanted off, and the solid dried in vacuo overnight. The dried material was collected and massed. Yield was found to be nearly stoichiometric.

### Example 8

### Synthesis of a 1:2 ZnG3 Chelate

A 1.0051 g sample of triglycine (G3—5.29 mmol; 1 eq.) was dissolved in approximately 10 mLs DI H₂O in a 50 mL round-bottom flask, with constant heating and stirring at 60° C. A separate solution of 361.6 mgs zinc chloride (ZnCl₂—2.64 mmol; 1 eq.) was taken up in approximately 10 mLs DI H₂O, constantly stirred and heated to 60° C. The ZnCl₂ solution was added to the triglycine solution—upon addition, the combined solution was colorless. The reaction was left to run for 1 hr at 60° C. The reaction was cooled to room temperature and filtered through a Buchner funnel (no solid was observed on the filter paper). The pH of the solution was found to be 5.84. The solution was concentrated down to approximately 3 mLs and the solid was precipitated with isopropanol. Centrifugation was employed to pellet the solid, and the isopropanol was decanted off. The solid was washed with diethyl ether to remove the isopropanol. The sample was then recentrifuged, the ether decanted off, and the solid dried in vacuo overnight. The dried material was collected and massed. Yield was found to be nearly stoichiometric.

### Example 9

### Zinc G3 Chelate Analysis

NMR analyses suggest reaction completion in as little as 10 minutes. NMR analysis coupled with IR, elemental analysis, and TGA/DSC suggest a tetrahedral diaqua zinc triglycine species with coordination occurring through the carboxylic acid (1:1). Chloride is believed to be the anion present as is suggested by the TGA/DSC analyses. Water solubility of zinc triglycine (46.6 g/100 mL) is greater than that of zinc acetate, but less than that of zinc sulfate. Cellular uptake, as elucidated by colorimetric assay, shows that ZnG3 has uptake comparable to that of both salts ZnCl₂ and Zn(NO₃)₂. Specifically, ZnG3 and Zn(NO₃)₂ show comparable cellular uptake with similar percent zinc composition. NMR, TGA/DSC and elemental analysis suggest an octahedral diaquo bistriglycine zinc complex (1:2).

### Example 10

### Synthesis of a Mg Di-Glycine Chelate

The general synthetic procedure for producing magnesium diglycine ([Mg(G2)⁻(H₂O)₃]OH⁻—MgG2) utilizes both magnesium oxide (MgO) and the monoacid dipeptide—diglycine (G2). The starting materials are combined in a 1:1 stoichiometry in the presence of acetic acid at 1 eq. and reacted at 90° C. for 1 hr in enough water such that the solution is wholly soluble (i.e. 1 g of G2 in 50 mLs, 5 g of G2 in 250 mLs, etc.). In certain embodiments, citric acid was also used to aid in reaction solubility. Upon reacting, the solution is analyzed for pure product via ¹H NMR. After product confirmation, the reaction is reduced to a minimum volume and MgG2 is precipitated as a white solid with anhydrous ethanol. Upon precipitation, the white solid is pelleted down via centrifugation and isolated by decanting off supernatant ethanol. The white solid is then treated and resuspended with copious diethyl ether and triturated to remove ethanol—further centrifugation is employed, the diethyl ether supernatant is decanted off and the retained solid is dried in vacuo overnight—final product is retained as a white solid. This synthetic approach results in a near stoichiometric yield and has been shown to be scalable to both 5 g and 50 g. Typical reaction pH is between 10-10.5.

### Example 11

### Characterization of a Mg Di-Glycine Chelate

Both solution-state and solid-state characterization were performed. Solution-state characterization of MgG2 includes product confirmation via ¹H/¹³C nuclear magnetic resonance (NMR), two dimensional (2D) heteronuclear single quantum coherence (HSQC) and heteronuclear multiple bond correlation (HMBC), and electrospray ionization mass spectrometry (ESI-MS). Solid-state characterization of MgG2 includes product confirmation via Fourier transform infrared radiation (FT-IR), thermogravimetric analysis (TGA) and differential scanning calorimetry (DSC), and elemental analysis (EA).

### Characterization of MgG2 via ¹H NMR

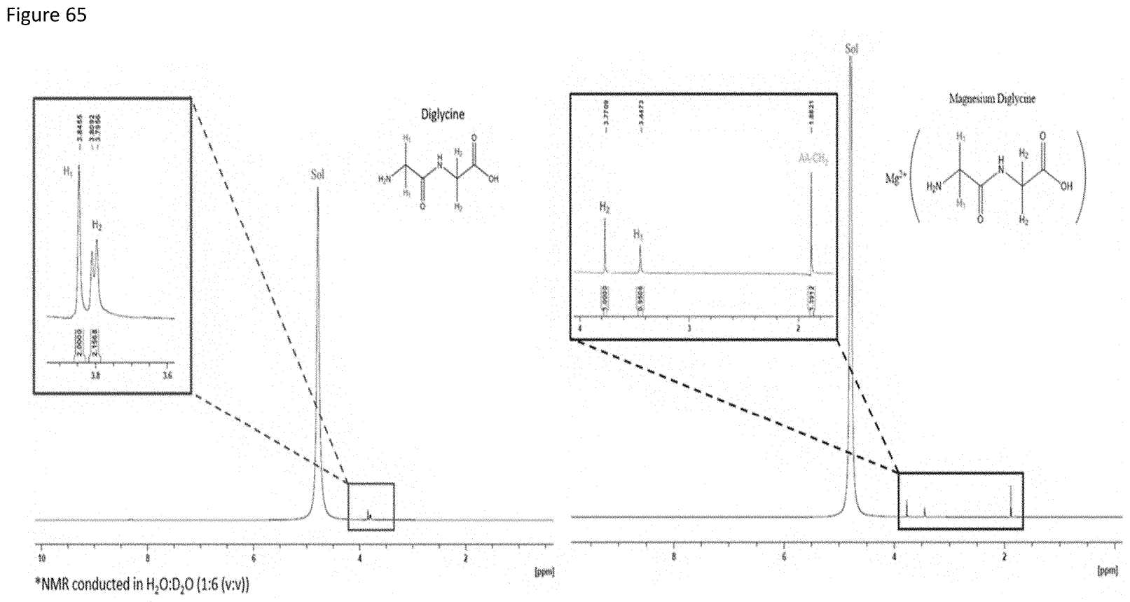

Upfield-shifting of organic ligand proton signals is a common observable with regard to ligand coordination to magnesium, and upon coordination of diglycine to magnesium, there is an observable upfield shift, and a change in the splitting pattern, of the diglycine protons, suggesting a change in electronic environment of those protons that is consistent with other magnesium organic-inorganic hybrid molecules upon coordination (FIG. 65).

Most notable are the observed change in splitting of the H₂ proton nearest the terminal acid and the significant 0.4 ppm upfield shift of the H₁ proton. The splitting of the H₂ proton of the free ligand diglycine is attributed to ability of the acid to participate in hydrogen bonding that is not available given the ligand's anionic nature during reaction at alkaline pH. This is also the result of predicted coordination through the acid upon deprotonation—further eliminating the ability of the acid to be protonated and participate in hydrogen bonding. The change in splitting of this proton from a doublet to a singlet is indicative of diglycine coordination to magnesium via the acid. The significant upfield shift of the H₁ proton suggests coordination through either the terminal amine or the backbone amine—although it is predicted that both participate given the entropic favorability of diglycine acting as a tridentate ligand as opposed to a bidentate ligand.

Furthermore, comparatively, the integration of the ¹H NMR of both diglycine and MgG2 is conserved—indicating almost no presence of impurity. The subsequent proton signal observed in the MgG2 spectrum is attributed to the methyl group (—CH₃) of acetic acid; this is the result of the 1 eq. of acetic acid required for reaction solubility. Utilization of spray drying is believed to remove the volatile acetic acid, thus resulting in pure, stand-alone MgG2.

### Characterization of MgDG via ¹³C NMR

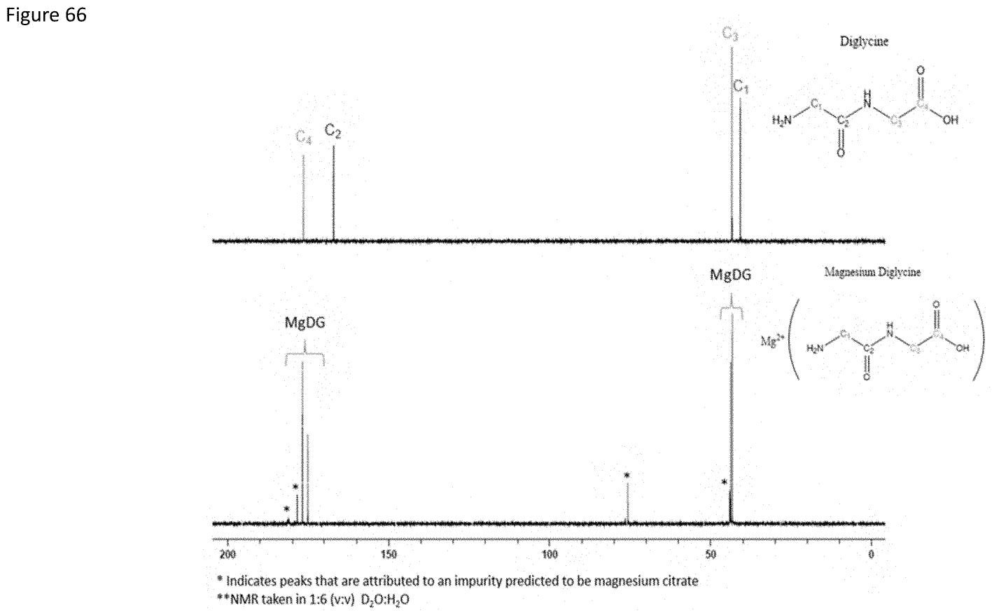

Utilization of ¹³C NMR provides further insight into the coordination of diglycine to magnesium. Just as ligand coordination to magnesium results in an observed shift of the ligand proton signals, the same is expected for the carbon signals; an observable shift is anticipated and is subsequently observed (FIG. 66).

The most upfield carbon signals are attributed the saturated diglycine carbons (C₁ and C₃) and the most downfield carbon signals are attributed to the unsaturated terminal acid carbon (C₄) and the backbone amide (C₂). There is an observed shift of the carbon signals that is only expected upon coordination to the magnesium given a change in the electronic environment of the carbons. Assignment of the carbon signals was expounded upon for further coordinative insight utilizing both 2D HSQC and HMBC.

### Characterization of MgG2 via 2-Dimensional HSQC and HMBC

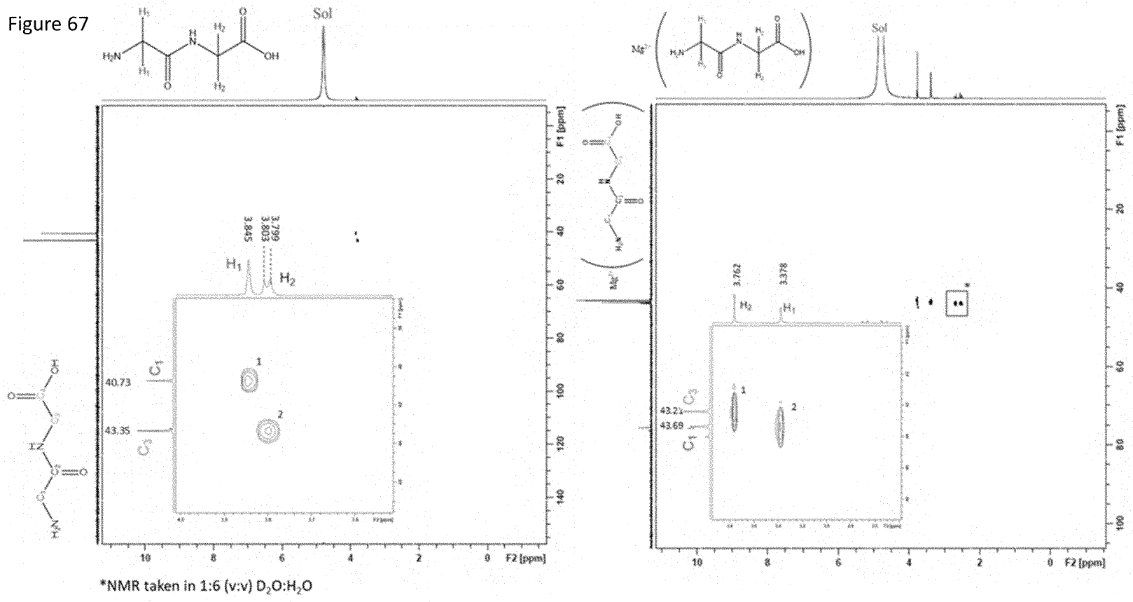

2D HSQC was utilized to confirm carbon signal assignments of diglycine based on proximity to proton signals—more specifically, HSQC elucidated the carbons that are only one bond distance away from a specific proton. In the case of diglycine, both H₁ and H₂ showed a single correspondence to one carbon signal (FIG. 67).

As expected, the HSQC elucidates the identities of both the saturated carbons—the HSQC also illustrates the observed downfield shift of the C₁ carbon. This supplies further confirmatory evidence of coordination participation by the terminal amine or the backbone amide as it compliments the observed upfield shift of the H₁ proton, again providing support for a change in the electronic environment about this region.

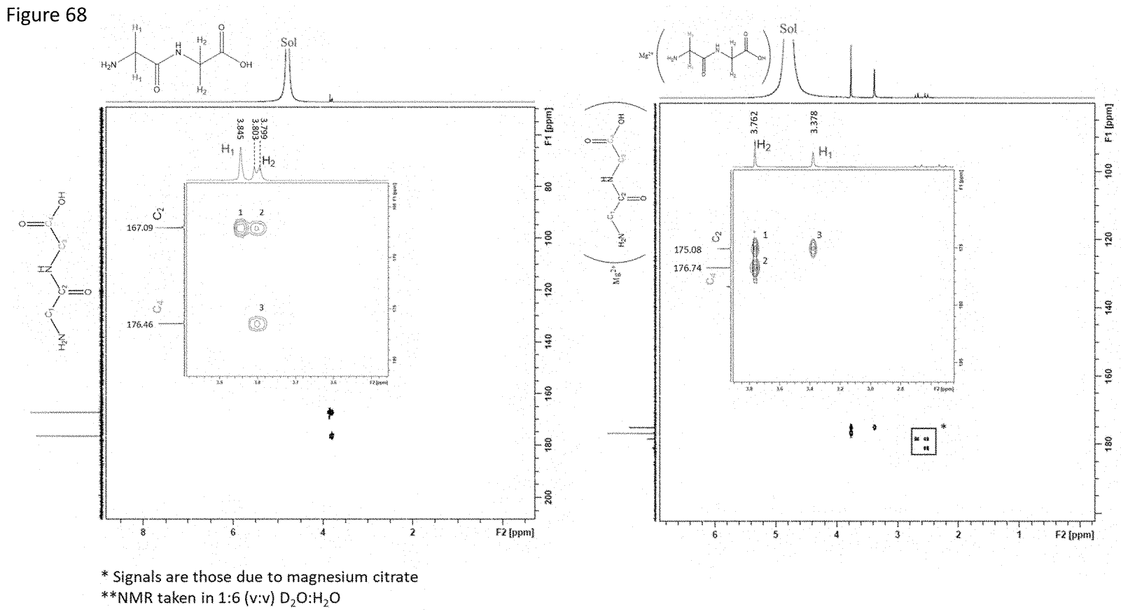

2D HMBC was utilized to confirm the identities of the terminal acid carbon as well as the backbone amide carbon by providing insight to the carbon environments that are two or more bonds away from proton environment of interest. In the case of diglycine, it was predicted that Hi would show only one correspondence point given a two-bond distance to the backbone amide, whereas H₂ was predicted to show two correspondence points given a two-bond distance to the terminal acid and a three-bond distance to the backbone amide. This prediction was confirmed via the HMBC (FIG. 68).

As illustrated by the HMBC, predictions for the number of correspondences were confirmed. The overlap of the correspondences of H₁ and H₂ at 175.08 ppm, confirm that this is C₂, as this is the only carbon that is within distance of both protons. Subsequently, there is an observed downfield shift of the C₂, which again illustrates the change in the electronic environment surrounding the terminal amine and the backbone amide which supports participation of these moieties in magnesium coordination—moreover, given differences in the proximity of C₂ relative to these moieties, this observation provides more support for the participation of the backbone amide, as the electronic environment would be more impacted by coordination to the backbone amide. This provides further insight into the diglycine ligand acting as a tridentate chelate of magnesium.

### Characterization of MgG2 via ESI-MS

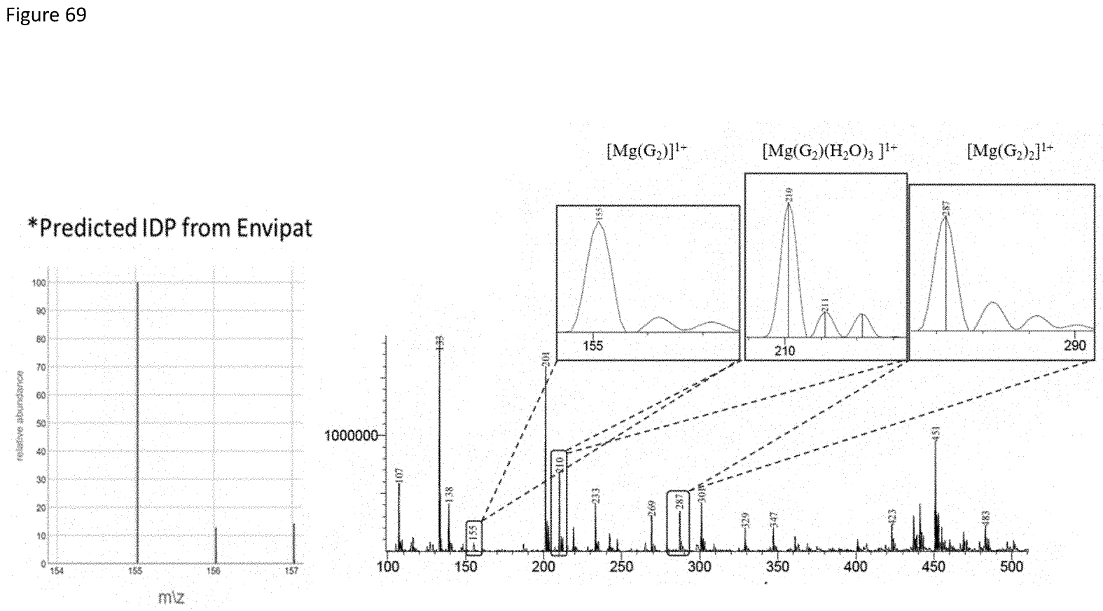

Electrospray ionization mass spectrometry was utilized to confirm the identity of MgG2 by analyzing the isotopic distribution pattern (IDP) as well as verifying that the observed mass-to-charge ratio was consistent with that which was predicted, subsequently confirming the predicted charge. The predicted stoichiometry of the complex was 1:1 given a 1:1 synthetic stoichiometry. Anticipating the tridentate coordination of the diglycine ligand, it is believed that the remaining coordination sites would be occupied by water, and as a result of the pH that the charge subsequent charge balance would be supplied by a hydroxy anion. The predicted mass of a the triaquamonodiglycine magnesium hydroxate complex was 226.4 g/mol, and the predicted mass of a strict 1:1 Mg:G2 complex was 155.4 g/mol with a mono-ionized magnesium species resulting in the same observed mass-to-charge (m/z) value (FIG. 69).

As predicted, the ESI-MS of MgG2 showed four peaks of interest: 133 m/z, 155 m/z, 210 m/z, and 287 m/z. Most notably, the peaks at 155 m/z and 210 m/z support the predicted 1:1 stoichiometry of the MgG2 complex, with the peak at 210 m/z also supporting the occupation of the additional three coordination sites by water. While the ESI-MS also shows the presence of the free diglycine ligand and a 1:2 Mg:G2 complex at 133 m/z and 287 m/z, respectively, the ¹H NMR shows no support that any species other than the 1:1 MgG2 complex exists in the solution state as there is no evidence of any protons existing in multiple environments such as a change in splitting pattern, or that there is any presence of the free diglycine ligand. This further supports the near stoichiometric yield of the MgG2 complex and provides further support for the purity of the complex.

### Characterization of MgG2 via FT-IR

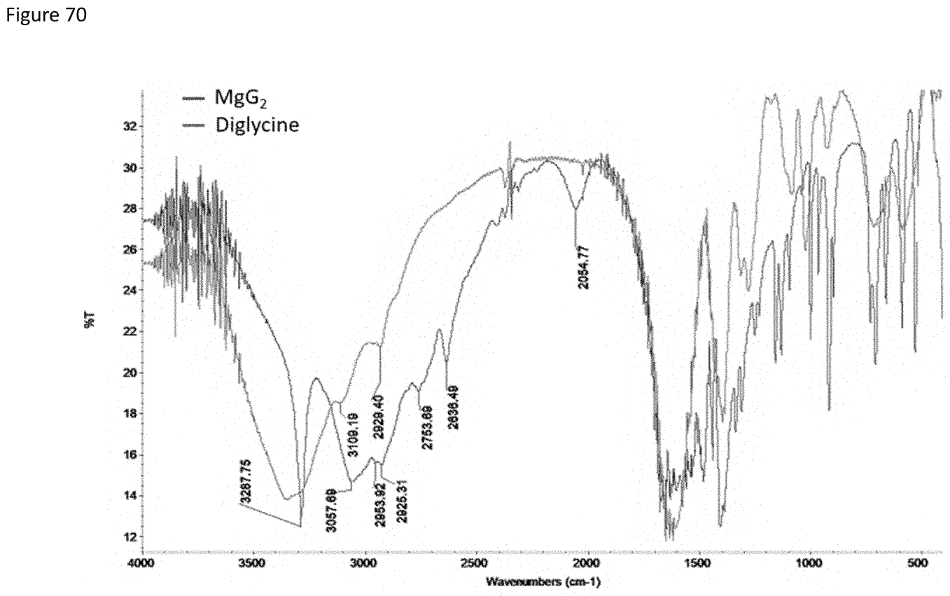

FT-IR was utilized to further confirm which diglycine moieties participated in magnesium coordination to supplement the sound predictions provided by the ¹H and ¹³C NMR. Functional groups of interest were the terminal acid, terminal amine, and backbone amide. It was predicted that the acid would deprotonate resulting in the subsequently non-observation of the acid proton. It was further hypothesized that both the backbone amide and the terminal amine would exhibit a subsequent change in dipole moment given the propensity of diglycine to act as a tridentate ligand. Use of FT-IR subsequently confirmed these predictions (FIG. 70).

The most distinct difference between the FT-IR spectra of the free diglycine ligand and MgG2 is the disappearance of the sharp peak at 3288 cm-1 from diglycine to MgG2. This sharp peak is attributed to the carboxylic acid proton of the free diglycine that is no long present in coordinated diglycine. This is consistent with predicted deprotonation and subsequent coordination of the free diglycine ligand about the free acid. Additionally, the disappearance of the band at 2637 cm-1 is also attributed to a change in the carboxylic acid moiety. Lastly, disappearance of the broad band at 2055 cm-1 from diglycine to magnesium diglycine is attributed to the coordination of the terminal amine and subsequent deprotonation of a free diglycine —NH₃_{⁺} moiety. These FT-IR findings are consistent with the prediction that the diglycine ligand utilizes all Lewis base moieties, via an N₂O donor set, and acts as a tridentate chelate.

### Characterization of MgG2 via TGA/DSC

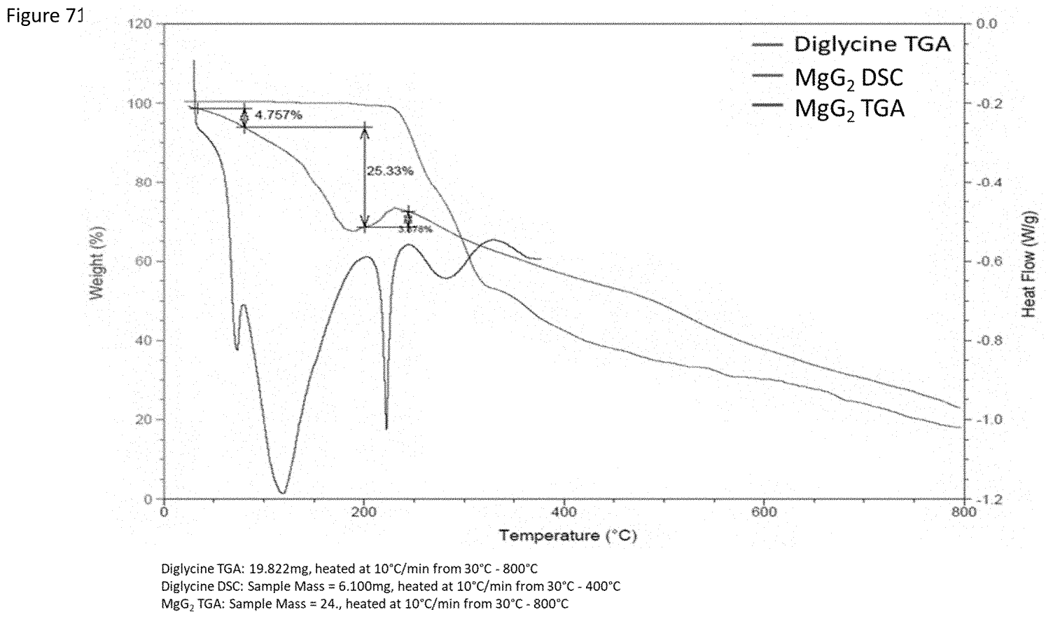

Given the tendency of magnesium to readily take on hydration water, with an extensive hydration sphere that far surpasses that of calcium, it was predicted that all magnesium coordination sites not occupied by the tridentate chelate diglycine would be occupied by coordinate water. To evaluate the degree of hydration of MgG2, both TGA and DSC were utilized. Both the TGA/DSC spectrum are shown below (FIG. 71).

As observed, the DSC of MgG2 shows three endotherms; endotherms are common to magnesium chelates. The relatively small endotherm at 78° C. is residual ethanol. The second observed endotherm apexed at approximately 120° C. is believed to be coordinated water and corresponds to the loss of three water from a triaqua-MgG2 complex with a 1:1 Mg:G2 stoichiometry. The endotherm observed at 220° C. is attributed to the decomposition of the diglycine ligand as is confirmed by the diglycine control. Subsequently, decomposition is more gradual for the MgG2 complex which suggests increased stability due to complexation. In general, analysis of MgG2 via TGA/DSC confirms that the coordination sites of MgG2 not occupied by the tridentate diglycine ligand are, in fact, occupied by water. This confirms an overall chemical composition of [Mg(G2-)(H₂O)₃]OH.

### Characterization of MgG2 via Elemental Analysis:

Characterization of MgG2 via elemental analysis was used for subsequent confirmation of the stoichiometry of the MgG2 complex. Analysis of elemental data confirms the 1:1 Mg:G2 stoichiometry of the overall MgG2 complex (see table below).

Elemental Analysis Values

| MgDG (1:1) | Carbon % | Nitrogen % | Hydrogen % |
|---|---|---|---|
| Calculated Values | 30.71% | 17.91% | 5.15% |
| Sample 1 | 30.43% | 17.00% | 5.19% |
| Sample 2 | 30.11% | 17.00% | 5.16% |
| Average Mass % | 30.27% | 17.00% | 5.18% |

As shown, the elemental analysis supports the confirmation that the MgDG complex retains a 1:1 Mg:G2 stoichiometry but does not provide any further information as to the degree of hydration of the compound.

### Example 12

### Cellular Uptake in Colorectal Carcinoma (CaCo-2) Cells:

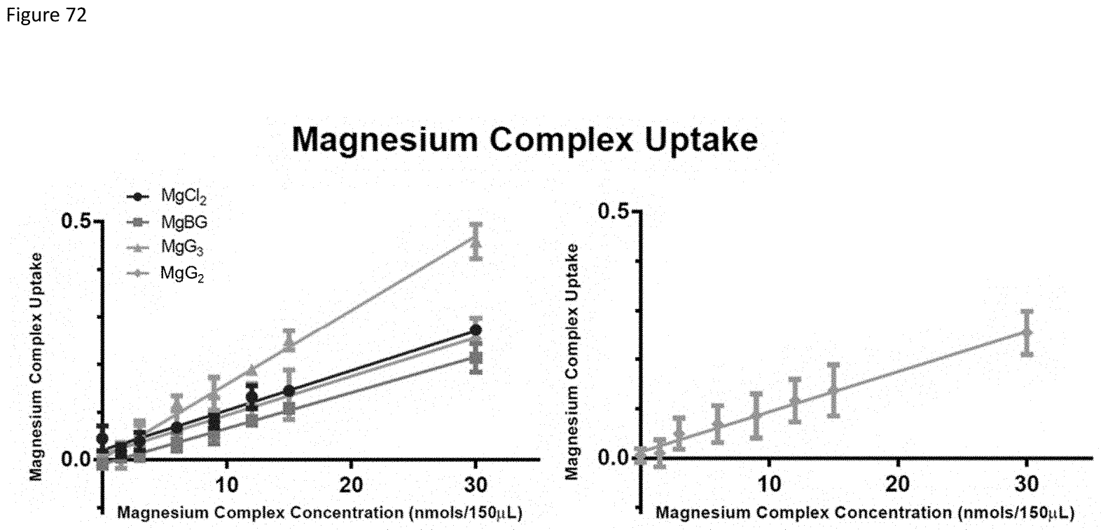

Cellular uptake of MgG2 was evaluated in the lower gastro intestinal (GI) model colorectal carcinoma (CaCo-2) cell line—as a model for cellular uptake in the lower GI (where the majority of magnesium uptake has been shown to take place). It has been illustrated that there is a strong correlation between the solubility of the magnesium complex and the resulting cellular uptake of the magnesium complex, as was illustrated previously with the use of magnesium triglycine (Mg(G3)). Subsequently, the solubility of MgG2 was evaluated, and the resulting cellular uptake relative to other common magnesium complexes analyzed (FIG. 72).

As shown, MgG2 shows relatively linear uptake in the CaCo-2 cell line similar to that of both magnesium chloride (MgCl2) and magnesium bisglycinate (MgBG). This similarity in cellular uptake is expected given the similarity in solubility of the complexes. Additionally, MgBG shows significantly less cellular uptake than Mg(G3), which again is expected given the significant discrepancy in solubility between the two. It is believed that this cellular uptake in vitro will correspond to increased bioavailability in vivo.

What is claimed is:

1. A composition comprising a magnesium di-amino acid chelate complex, the complex comprising a magnesium di-amino acid chelate and a counterion, wherein there is a 1:1 ratio between the magnesium and the di-amino acid.

2. The composition of claim 1, wherein the di-amino acid is selected from the group consisting of di-glycine (G2), di-aspartic acid (D2), di-glutamic acid (E2), di-histidine (H2), di-serine (S2), and di-tyrosine (Y2).

3. The composition of claim 1, wherein each amino acid of the di-amino acid is selected from the group consisting of glycine (G), aspartic acid (D), glutamic acid (E), histidine (H), serine (S), and tyrosine (Y).

4. The composition of claim 1, wherein the di-amino acid is di-glycine.

5. The composition of claim 1, further comprising at least one vitamin or additional mineral.

6. A composition comprising a zinc di-amino acid chelate complex, the complex comprising a zinc di-amino acid chelate and optionally a counterion, wherein there is a 1:1 or 1:2 ratio between the zinc and the di-amino acid.

7. The composition of claim 6, wherein the di-amino acid is selected from the group consisting of di-glycine (G2), di-aspartic acid (D2), di-glutamic acid (E2), di-histidine (H2), di-serine (S2), and di-tyrosine (Y2).

8. The composition of claim 6, wherein each amino acid of the di-amino acid is selected from the group consisting of glycine (G), aspartic acid (D), glutamic acid (E), histidine (H), serine (S), and tyrosine (Y).

9. The composition of claim 6, wherein the di-amino acid is di-glycine.

10. The composition of claim 6, further comprising at least one vitamin or additional mineral.

11. A composition comprising a calcium di-amino acid chelate complex, the complex comprising a calcium di-amino acid chelate and optionally a counterion, wherein there is a 1:1 or 1:2 ratio between the calcium and the di-amino acid.

12. The composition of claim 11, wherein the di-amino acid is selected from the group consisting of di-glycine (G2), di-aspartic acid (D2), di-glutamic acid (E2), di-histidine (H2), di-serine (S2), and di-tyrosine (Y2).

13. The composition of claim 11, wherein each amino acid of the di-amino acid is selected from the group consisting of glycine (G), aspartic acid (D), glutamic acid (E), histidine (H), serine (S), and tyrosine (Y).

14. The composition of claim 11, wherein the di-amino acid is di-glycine.

15. The composition of claim 11, further comprising at least one vitamin or additional mineral.

16. A pharmaceutical formulation, the formulation comprising the composition of any of claim 1.

17. The pharmaceutical formulation of claim 16, wherein the formulation is suitable for oral administration.

18. The pharmaceutical formulation of claim 16, wherein the formulation is suitable for parenteral administration.

19. A method of supplying a metal to a subject deficient in a metal, the method comprising administering the chelate of claim 1 to the subject.

20. The method of claim 19, wherein the metal is selected from the group consisting of magnesium, calcium, zinc, iron, and a mixture thereof.

21. A method of producing a metal di-amino acid chelate complex, the method comprising
(i) creating an aqueous solution of a di-amino acid, a metal compound, and optionally an organic acid,
(ii) stirring the solution of step (i) while optionally heating up to 90° C. for a minimum of 10 min,
(iii) cooling the solution and precipitating the metal di-amino acid chelate complex.

22. The method of claim 21, the method comprising individually heating an aqueous solution of the di-amino acid and an aqueous solution of the metal compound and optionally organic acid before combining them to create the solution of step (i).

23. A metal di-amino acid chelate complex formed by the method comprising
(i) creating an aqueous solution of a di-amino acid, a metal compound, and optionally an organic acid,
(ii) stirring the solution of step (i) while optionally heating the solution to less than 90° C. for a minimum of 10 min,
(iii) cooling the combination and precipitating the metal di-amino acid chelate complex.
